(19) United States
(12) Patent Application Publication
(10) Pub. No.: US 2021/0253691 A1
(43) Pub. Date: Aug. 19, 2021

(54) METHODS FOR IMPROVING RESPONSE TO ANTI-LIF ANTIBODY TREATMENT IN INDIVIDUALS WITH CANCER

(71) Applicants: MEDIMMUNE LIMITED, Cambridge (GB); FUNDACIO PRIVADA INSTITUT D'INVESTIGACIO ONCOLOGICA DE VALL HEBRON, Barcelona (ES); FUNDACIO PRIVADA INSTITUCIO CATALANA DE RECERCA 1 ESTUDIS AVANCATS, Barcelona (ES)

(72) Inventors: Joan SEOANE SUAREZ, Barcelona (ES); Judit ANIDO FOLGUEIRA, Barcelona (ES); Robin Matthew HALLETT, Toronto, ON (CA); Peter Edward BAYLISS, Toronto, ON (CA); Ajitha JEGANATHAN, Toronto, ON (CA); Patricia Anne GIBLIN, Boston, MA (US); Isabel Huber RUANO, Barcelona (ES); Jeanne MAGRAM, Boston, MA (US); Monica Pascual GARCIA, Barcelona (ES); Ester Bonfill TEIXIDOR, Barcelona (ES); Ester Planas RIGOL, Barcelona (ES)

(21) Appl. No.: 17/252,482

(22) PCT Filed: Jun. 17, 2019
(86) PCT No.: PCT/IB2019/000806

§ 371 Date: Dec. 15, 2020

(30) Foreign Application Priority Data
Jun. 18, 2018 (EP) 18382431.7
Feb. 22, 2019 (EP) 19382131.1

(51) Int. Cl. C07K 16/24; A61P 35/00; G01N 33/574

(52) U.S. Cl. CPC C07K 16/244; A61K 2039/505; G01N 33/57488; A61P 35/00

(57) ABSTRACT

Described herein are methods of selecting and treating patients likely to respond to treatment with an anti-LIF antibody.

### CROSS REFERENCE TO RELATED APPLICATIONS

The present application claims the benefit of European Application Serial Number 18382431.7, filed Jun. 18, 2018, and European Application Serial Number 19382131.1, filed Feb. 22, 2019, all of which are hereby incorporated by reference in their entireties.

### BACKGROUND

Leukemia inhibitory factor (LIF) is a member of the interleukin-6 (IL-6) family of cytokines. Based on the known physiological activities of LIF, clinical features of LIF deficiency, and nonclinical studies of LIF expression in healthy tissues and tumors, inhibition of LIF is expected to be well tolerated and to result in anti-tumor efficacy in a range of solid tumors, including but not limited to non-small cell lung cancer (NSCLC), pancreatic cancer, ovarian cancer and glioblastoma multiforme (GBM).

One problem facing clinicians when treating cancer with target specific therapeutics, is that not all tumors may be responsive to a given target specific therapeutic. Even for tumor types known to express LIF or the LIF receptor there is significant heterogeneity amongst individual tumors. Additionally, not all tumors that express LIF or LIF receptor may respond similarly, thus there is a need for effective methods to determine individuals that may most benefit from treatment with an anti-LIF therapeutic antibody.

### SUMMARY

The present disclosure relates to methods of treating cancer in individuals comprising administering a therapeutic anti-LIF antibody to those individuals most likely to respond to said antibody, and methods of determining which individuals are most likely to respond to a therapeutic anti-LIF antibody. Patients with tumors or cancers that exhibit expression of LIF or the LIF receptor at an mRNA or protein level that exceeds a reference level, as described herein, can be effectively treated with a LIF therapeutic antibody. Additionally, several non-LIF biomarkers are described that can determine individuals that would benefit from treatment with a therapeutic anti-LIF antibody. These non-LIF biomarkers can be used alone or together with an assay that measures a LIF or a LIF receptor level. Non-LIF biomarkers include immunomodulatory molecules that indicate an immunosuppressive signature, these include, the presence of immunosuppressive cell types, immunosuppressive cytokines, or immunosuppressive chemokines. It is envisioned that determining a LIF or LIF receptor level together with an immunosuppressive signature will increase the predictive power of a method of determining treatment with a therapeutic anti-LIF antibody.

In one aspect, described herein, is a method of treating an individual with cancer with a therapeutic anti-leukemia inhibitory factor (LIF) antibody comprising determining a level of LIF that exceeds a reference level in a biological sample from the individual, and administering a therapeutic amount of the anti-LIF antibody to the individual when the level of LIF is greater than the reference level of LIF. In certain embodiments, the therapeutic anti-LIF antibody comprises: an immunoglobulin heavy chain complementarity determining region 1 (VH-CDR1) comprising the amino acid sequence set forth in any one of SEQ ID NOs: 1-3; an immunoglobulin heavy chain complementarity determining region 2 (VH-CDR2) comprising the amino acid sequence set forth in any one of SEQ ID NOs: 4 or 5; an immunoglobulin heavy chain complementarity determining region 3 (VH-CDR3) comprising the amino acid sequence set forth in any one of SEQ ID NOs: 6-8; an immunoglobulin light chain complementarity determining region 1 (VL-CDR1) comprising the amino acid sequence set forth in any one of SEQ ID NOs: 9 or 10; an immunoglobulin light chain complementarity determining region 2 (VL-CDR2) comprising the amino acid sequence set forth in any one of SEQ ID NOs: 11 or 12; and an immunoglobulin light chain complementarity determining region 3 (VL-CDR3) comprising the amino acid sequence set forth in SEQ ID NO: 13. In certain embodiments, the therapeutic anti-LIF antibody comprises an immunoglobulin heavy chain variable region comprising at least 85%, 90%, 95%, 97%, 98%, 99%, or 100% identity to SEQ ID NOs: 14, 15, 17 or 38 and an immunoglobulin light chain variable region comprising at least 85%, 90%, 95%, 97%, 98%, 99%, or 100% identity to SEQ ID NO: 18-21. In certain embodiments, the therapeutic anti-LIF antibody comprises an immunoglobulin heavy chain region comprising at least 85%, 90%, 95%, 97%, 98%, 99%, or 100% identity to SEQ ID NOs: 30-33 or 39, and an immunoglobulin light chain region comprising at least 85%, 90%, 95%, 97%, 98%, 99%, or 100% identity to SEQ ID NOs: 34-37. In certain embodiments, the therapeutic anti-LIF antibody is an IgG antibody comprising two immunoglobulin heavy chains and two immunoglobulin light chains. In certain embodiments, the level of LIF is a LIF protein level and determining the level comprises performing at least one assay that detects LIF protein or receiving the results of at least one assay that detects LIF protein. In certain embodiments, the at least one assay comprises immunohistochemistry. In certain embodiments, the reference level is about 1%, 2%, 3%. 4%, 5%, 6%, 7%, 8%, 9%, 10%, 15%, 20%, 25%, 30%, 35%, or 50% of cells staining positive with an anti-LIF antibody.

In certain embodiments, the reference level is an IHC-score of about 100. In some embodiments, the reference level is an IHC-score of about 1 to about 300. In some embodiments, the reference level is an IHC-score of about 1 to about 30, about 1 to about 60, about 1 to about 90, about 1 to about 120, about 1 to about 150, about 1 to about 180, about 1 to about 210, about 1 to about 240, about 1 to about 270, about 1 to about 300, about 30 to about 60, about 30 to about 90, about 30 to about 120, about 30 to about 150, about 30 to about 180, about 30 to about 210, about 30 to about 240, about 30 to about 270, about 30 to about 300, about 60 to about 90, about 60 to about 120, about 60 to about 150, about 60 to about 180, about 60 to about 210, about 60 to about 240, about 60 to about 270, about 60 to about 300, about 90 to about 120, about 90 to about 150, about 90 to about 180, about 90 to about 210, about 90 to about 240, about 90 to about 270, about 90 to about 300, about 120 to about 150, about 120 to about 180, about 120 to about 210, about 120 to about 240, about 120 to about 270, about 120 to about 300, about 150 to about 180, about 150 to about 210, about 150 to about 240, about 150 to about 270, about 150 to about 300, about 180 to about 210, about 180 to about 240, about 180 to about 270, about 180 to about 300, about 210 to about 240, about 210 to about 270, about 210 to about 300, about 240 to about 270, about 240 to about 300, or about 270 to about 300. In some embodiments, the reference level is an IHC-score of about 1, about 30, about 60, about 90, about 120, about 150, about 180, about 210, about 240, about 270, or about 300. In some embodiments, the reference level is an IHC-score of at least about 1, about 30, about 60, about 90, about 120, about 150, about 180, about 210, about 240, or about 270. In some embodiments, the reference level is an IHC-score of at most about 30, about 60, about 90, about 120, about 150, about 180, about 210, about 240, about 270, or about 300. In some embodiments, the reference level is or exceeds an IHC-score of about 10 to about 100.

In certain embodiments, at least one assay comprises an enzyme linked immunosorbent assay (ELISA). In certain embodiments, the ELISA detects electrochemiluminescence. In certain embodiments, the reference level is about 1 pg/mL to about 10 pg/mL of LIF in an undiluted biological sample from the individual. In certain embodiments, the reference level of LIF corresponds to the 5^{th} percentile of LIF protein expression in LIF positive cancers of the same type. In certain embodiments, the reference level of LIF corresponds to the 10^{th} percentile of LIF protein expression in LIF positive cancers of the same type. In certain embodiments, the reference level of LIF corresponds to the 5^{th} percentile of LIF protein expression in a representative sample of human cancers. In certain embodiments, the reference level of LIF corresponds to the 10^{th} percentile of LIF protein expression in a representative sample of human cancers. In certain embodiments, the level of LIF is a LIF mRNA level and determining the level comprises performing at least one assay that detects LIF mRNA or receiving the results of at least one assay that detects LIF mRNA. In certain embodiments, the reference level is a level corresponding to the 5^{th} percentile of LIF mRNA expression in cancers of the same type. In certain embodiments, the reference level is a level corresponding to the 10^{th} percentile of LIF mRNA expression in cancers of the same type. In certain embodiments, the reference level is a level corresponding to the 5^{th} percentile of LIF mRNA expression in a representative sample of human cancers. In certain embodiments, the reference level is a level corresponding to the 10^{th} percentile of LIF mRNA expression in a representative sample of human cancers. In certain embodiments, the reference level of LIF corresponds to the 25^{th} percentile of LIF protein expression in LIF positive cancers of the same type. In certain embodiments, the reference level of LIF corresponds to the 50^{th} percentile of LIF protein expression in LIF positive cancers of the same type. In certain embodiments, the reference level of LIF corresponds to the 25^{th} percentile of LIF protein expression in a representative sample of human cancers. In certain embodiments, the reference level of LIF corresponds to the 50^{th} percentile of LIF protein expression in a representative sample of human cancers. In certain embodiments, the level of LIF is a LIF mRNA level and determining the level comprises performing at least one assay that detects LIF mRNA or receiving the results of at least one assay that detects LIF mRNA. In certain embodiments, the reference level is a level corresponding to the 25^{th} percentile of LIF mRNA expression in cancers of the same type. In certain embodiments, the reference level is a level corresponding to the 50^{th} percentile of LIF mRNA expression in cancers of the same type. In certain embodiments, the reference level is a level corresponding to the 25^{th} percentile of LIF mRNA expression in a representative sample of human cancers. In certain embodiments, the reference level is a level corresponding to the 50^{th} percentile of LIF mRNA expression in a representative sample of human cancers. In certain embodiments, the level of LIF is a LIF DNA level and determining the level comprises performing at least one assay that detects LIF DNA or receiving the results of at least one assay that detects LIF DNA. In certain embodiments, at least one assay comprises polymerase chain reaction (PCR). In certain embodiments, the PCR comprises quantitative PCR. In certain embodiments, the at least one assay comprises a sequencing reaction. In certain embodiments, the sequencing reaction comprises a next-generation sequencing reaction. In certain embodiments, the biological sample comprises a blood sample. In certain embodiments, the blood sample is plasma. In certain embodiments, the blood sample is serum. In certain embodiments, the biological sample comprises a tissue sample. In certain embodiments, the biological sample is a tumor biopsy. In certain embodiments, the method further comprises determining a DNA, mRNA, or protein level of an immunomodulatory molecule that exceeds a reference level of the immunomodulatory molecule. In certain embodiments, the method further comprises determining a DNA, mRNA, or protein level of an immunomodulatory molecule that is below a reference level of the immunomodulatory molecule. In certain embodiments, the immunomodulatory molecule is selected from an mRNA transcribed from or a protein produced from the list consisting of MHCII, CXCL9, CXCL10, CXCR3, PD-L1, CCL7, CCL2, CCL3, and CCL22. In certain embodiments, the method further comprises determining a level of a Type II macrophage (M2) marker that exceeds a reference level of DNA, mRNA, or protein of the Type II macrophage (M2) marker. In certain embodiments, the M2 marker is an mRNA transcribed from or a protein produced from the list consisting of CD206, CD163, PF4, CTSK, and ARG1. In certain embodiments, the method further comprises determining a DNA, mRNA, or protein level of LIF receptor (LIFR) that exceeds a reference level of LIFR. In certain embodiments, the level of LIFR is detected on an immunomodulatory cell. In certain embodiments, the human cancer is selected from the list consisting of non-small cell lung cancer, ovarian cancer, kidney cancer, bladder cancer, pancreatic cancer, prostate cancer, genitourinary cancer, gynecologic cancer, gastrointestinal cancer, endocrine system cancer, glioblastoma multiforme, breast cancer, melanoma, colorectal cancer, bile duct cancer, cervical cancer, endometrial cancer, head and neck squamous cell carcinoma, and combinations thereof. In certain embodiments, the human cancer is selected from the list consisting of non-small cell lung cancer, ovarian cancer, kidney cancer, bladder cancer, and combinations thereof. In certain embodiments, the human cancer is selected from the list consisting of pancreatic cancer, prostate cancer, glioblastoma multiforme, and combinations thereof.

In another aspect, described herein, is a method of treating an individual with cancer with a therapeutic anti-leukemia inhibitory factor (LIF) antibody comprising determining a level of LIF that exceeds a reference level in a biological sample from the individual, and administering a therapeutic amount of the anti-LIF antibody. In certain embodiments, the therapeutic anti-LIF antibody comprises: an immunoglobulin heavy chain complementarity determining region 1 (VH-CDR1) comprising the amino acid sequence set forth in any one of SEQ ID NOs: 1-3; an immunoglobulin heavy chain complementarity determining region 2 (VH-CDR2) comprising the amino acid sequence set forth in any one of SEQ ID NOs: 4 or 5; an immunoglobulin heavy chain complementarity determining region 3 (VH-CDR3) comprising the amino acid sequence set forth in any one of SEQ ID NOs: 6-8; an immunoglobulin light chain complementarity determining region 1 (VL-CDR1) comprising the amino acid sequence set forth in any one of SEQ ID NOs: 9 or 10; an immunoglobulin light chain complementarity determining region 2 (VL-CDR2) comprising the amino acid sequence set forth in any one of SEQ ID NOs: 11 or 12; and an immunoglobulin light chain complementarity determining region 3 (VL-CDR3) comprising the amino acid sequence set forth in SEQ ID NO: 13. In certain embodiments, the therapeutic anti-LIF antibody comprises an immunoglobulin heavy chain variable region comprising at least 85%, 90%, 95%, 97%, 98%, 99%, or 100% identity to SEQ ID NOs: 14, 15, 17 or 38 and an immunoglobulin light chain variable region comprising at least 85%, 90%, 95%, 97%, 98%, 99%, or 100% identity to SEQ ID NO: 18-21. In certain embodiments, the therapeutic anti-LIF antibody comprises an immunoglobulin heavy chain region comprising at least 85%, 90%, 95%, 97%, 98%, 99%, or 100% identity to SEQ ID NOs: 30-33 or 39, and an immunoglobulin light chain region comprising at least 85%, 90%, 95%, 97%, 98%, 99%, or 100% identity to SEQ ID NOs: 34-37. In certain embodiments, the therapeutic anti-LIF antibody is an IgG antibody comprising two immunoglobulin heavy chains and two immunoglobulin light chains. In certain embodiments, the level of LIF is a LIF protein level and determining the level comprises performing at least one assay that detects LIF protein or receiving the results of at least one assay that detects LIF protein. In certain embodiments, the at least one assay comprises immunohistochemistry. In certain embodiments, the reference level is about 1%, 2%, 3%. 4%, 5%, 6%, 7%, 8%, 9%, 10%, 15%, 20%, 25%, 30%, 35%, or 50% of cells staining positive with an anti-LIF antibody.

In certain embodiments, the reference level is an IHC-score of about 100. In some embodiments, the reference level is an IHC-score of about 1 to about 300. In some embodiments, the reference level is an IHC-score of about 1 to about 30, about 1 to about 60, about 1 to about 90, about 1 to about 120, about 1 to about 150, about 1 to about 180, about 1 to about 210, about 1 to about 240, about 1 to about 270, about 1 to about 300, about 30 to about 60, about 30 to about 90, about 30 to about 120, about 30 to about 150, about 30 to about 180, about 30 to about 210, about 30 to about 240, about 30 to about 270, about 30 to about 300, about 60 to about 90, about 60 to about 120, about 60 to about 150, about 60 to about 180, about 60 to about 210, about 60 to about 240, about 60 to about 270, about 60 to about 300, about 90 to about 120, about 90 to about 150, about 90 to about 180, about 90 to about 210, about 90 to about 240, about 90 to about 270, about 90 to about 300, about 120 to about 150, about 120 to about 180, about 120 to about 210, about 120 to about 240, about 120 to about 270, about 120 to about 300, about 150 to about 180, about 150 to about 210, about 150 to about 240, about 150 to about 270, about 150 to about 300, about 180 to about 210, about 180 to about 240, about 180 to about 270, about 180 to about 300, about 210 to about 240, about 210 to about 270, about 210 to about 300, about 240 to about 270, about 240 to about 300, or about 270 to about 300. In some embodiments, the reference level is an IHC-score of about 1, about 30, about 60, about 90, about 120, about 150, about 180, about 210, about 240, about 270, or about 300. In some embodiments, the reference level is an IHC-score of at least about 1, about 30, about 60, about 90, about 120, about 150, about 180, about 210, about 240, or about 270. In some embodiments, the reference level is an IHC-score of at most about 30, about 60, about 90, about 120, about 150, about 180, about 210, about 240, about 270, or about 300.

In certain embodiments, at least one assay comprises enzyme linked immunosorbent assay (ELISA). In certain embodiments, the ELISA detects electrochemiluminescence. In certain embodiments, the reference level is at least about 4 pg/mL of LIF in an undiluted biological sample from the individual. In certain embodiments, the reference level of LIF corresponds to the 5^{th} percentile of LIF protein expression in LIF positive cancers of the same type. In certain embodiments, the reference level of LIF corresponds to the 10^{th} percentile of LIF protein expression in LIF positive cancers of the same type. In certain embodiments, the reference level of LIF corresponds to the 5^{th} percentile of LIF protein expression in a representative sample of human cancers. In certain embodiments, the reference level of LIF corresponds to the 10^{th} percentile of LIF protein expression in a representative sample of human cancers. In certain embodiments, the level of LIF is a LIF mRNA level and determining the level comprises performing at least one assay that detects LIF mRNA or receiving the results of at least one assay that detects LIF mRNA. In certain embodiments, the reference level is a level corresponding to the 5^{th} percentile of LIF mRNA expression in cancers of the same type. In certain embodiments, the reference level is a level corresponding to the 10^{th} percentile of LIF mRNA expression in cancers of the same type. In certain embodiments, the reference level is a level corresponding to the 5^{th} percentile of LIF mRNA expression in a representative sample of human cancers. In certain embodiments, the reference level is a level corresponding to the 10^{th} percentile of LIF mRNA expression in a representative sample of human cancers. In certain embodiments, the reference level of LIF corresponds to the 25^{th} percentile of LIF protein expression in LIF positive cancers of the same type. In certain embodiments, the reference level of LIF corresponds to the 50^{th} percentile of LIF protein expression in LIF positive cancers of the same type. In certain embodiments, the reference level of LIF corresponds to the 25^{th} percentile of LIF protein expression in a representative sample of human cancers. In certain embodiments, the reference level of LIF corresponds to the 50^{th} percentile of LIF protein expression in a representative sample of human cancers. In certain embodiments, the level of LIF is a LIF mRNA level and determining the level comprises performing at least one assay that detects LIF mRNA or receiving the results of at least one assay that detects LIF mRNA. In certain embodiments, the reference level is a level corresponding to the 25^{th} percentile of LIF mRNA expression in cancers of the same type. In certain embodiments, the reference level is a level corresponding to the 50^{th} percentile of LIF mRNA expression in cancers of the same type. In certain embodiments, the reference level is a level corresponding to the 25^{th} percentile of LIF mRNA expression in a representative sample of human cancers. In certain embodiments, the reference level is a level corresponding to the 50^{th} percentile of LIF mRNA expression in a representative sample of human cancers. In certain embodiments, the level of LIF is a LIF DNA level and determining the level comprises performing at least one assay that detects LIF DNA or receiving the results of at least one assay that detects LIF DNA. In certain embodiments, the at least one assay comprises polymerase chain reaction (PCR). In certain embodiments, the PCR comprises quantitative PCR. In certain embodiments, the at least one assay comprises a sequencing reaction. In certain embodiments, the sequencing reaction comprises a next-generation sequencing reaction. In certain embodiments, the biological sample comprises a blood sample. In certain embodiments, the blood sample is plasma. In certain embodiments, the blood sample is serum. In certain embodiments, the biological sample comprises a tissue sample. In certain embodiments, the biological sample is a tumor biopsy. In certain embodiments, the method further comprises determining a DNA, mRNA, or protein level of an immunomodulatory molecule that exceeds a reference level of the immunomodulatory molecule. In certain embodiments, the method further comprises determining a DNA, mRNA, or protein level of an immunomodulatory molecule that is below a reference level of the immunomodulatory molecule. In certain embodiments, the immunomodulatory molecule is selected from an mRNA transcribed from or a protein produced from the list consisting of MHCII, CXCL9, CXCL10, CXCR3, PD-L1, CCL7, CCL2, CCL3, and CCL22. In certain embodiments, the method further comprises determining a DNA, mRNA, or protein level of a Type II macrophage (M2) marker that exceeds a reference level of the Type II macrophage (M2) marker. In certain embodiments, the M2 marker is an mRNA transcribed from or a protein produced from the list consisting of CD206, CD163, PF4, CTSK, and ARG1. In certain embodiments, the method further comprises determining a DNA, mRNA, or protein level of LIF receptor (LIFR) that exceeds a reference level of LIFR. In certain embodiments, the level of LIFR is detected on an immunomodulatory cell. In certain embodiments, the human cancer is selected from the list consisting of non-small cell lung cancer, ovarian cancer, kidney cancer, bladder cancer, pancreatic cancer, prostate cancer, genitourinary cancer, gynecologic cancer, gastrointestinal cancer, endocrine system cancer, glioblastoma multiforme, breast cancer, melanoma, colorectal cancer, bile duct cancer, cervical cancer, endometrial cancer, head and neck squamous cell carcinoma, and combinations thereof. In certain embodiments, the human cancer is selected from the list consisting of non-small cell lung cancer, ovarian cancer, kidney cancer, bladder cancer, and combinations thereof. In certain embodiments, the human cancer is selected from the list consisting of pancreatic cancer, prostate cancer, glioblastoma multiforme, and combinations thereof.

In another aspect, described herein is a method of treating an individual with cancer with a therapeutic anti-Leukemia inhibitory factor (LIF) antibody comprising determining a level of Leukemia inhibitory factor receptor (LIFR) that exceeds a reference level in a biological sample from the individual, and administering a therapeutic amount of the anti-LIF antibody to the individual when the level of LIFR is greater than the reference level of LIFR. In certain embodiments, the level of LIFR is detected on an immunomodulatory cell. In certain embodiments, the therapeutic anti-LIF antibody comprises: an immunoglobulin heavy chain complementarity determining region 1 (VH-CDR1) comprising the amino acid sequence set forth in any one of SEQ ID NOs: 1-3; an immunoglobulin heavy chain complementarity determining region 2 (VH-CDR2) comprising the amino acid sequence set forth in any one of SEQ ID NOs: 4 or 5; an immunoglobulin heavy chain complementarity determining region 3 (VH-CDR3) comprising the amino acid sequence set forth in any one of SEQ ID NOs: 6-8; an immunoglobulin light chain complementarity determining region 1 (VL-CDR1) comprising the amino acid sequence set forth in any one of SEQ ID NOs: 9 or 10; an immunoglobulin light chain complementarity determining region 2 (VL-CDR2) comprising the amino acid sequence set forth in any one of SEQ ID NOs: 11 or 12; and an immunoglobulin light chain complementarity determining region 3 (VL-CDR3) comprising the amino acid sequence set forth in SEQ ID NO: 13. In certain embodiments, the therapeutic anti-LIF antibody comprises an immunoglobulin heavy chain variable region comprising at least 85%, 90%, 95%, 97%, 98%, 99%, or 100% identity to SEQ ID NOs: 14, 15, 17 or 38 and an immunoglobulin light chain variable region comprising at least 85%, 90%, 95%, 97%, 98%, 99%, or 100% identity to SEQ ID NO: 18-21. In certain embodiments, the therapeutic anti-LIF antibody comprises an immunoglobulin heavy chain region comprising at least 85%, 90%, 95%, 97%, 98%, 99%, or 100% identity to SEQ ID NOs: 30-33 or 39, and an immunoglobulin light chain region comprising at least 85%, 90%, 95%, 97%, 98%, 99%, or 100% identity to SEQ ID NOs: 34-37. In certain embodiments, the therapeutic anti-LIF antibody is an IgG antibody comprising two immunoglobulin heavy chains and two immunoglobulin light chains. In certain embodiments, the level of LIFR is a level of LIFR protein and determining the level comprises performing at least one assay that detects LIFR protein or receiving the results of at least one assay that detects LIFR protein. In certain embodiments, the at least one assay comprises immunohistochemistry. In certain embodiments, the at least one assay comprises enzyme linked immunosorbent assay (ELISA). In certain embodiments, the ELISA detects electrochemiluminescence. In certain embodiments, the at least one assay comprises flow cytometry. In certain embodiments, the level of LIFR is a level of LIFR mRNA and determining the level comprises performing at least one assay that detects LIFR mRNA or receiving the results of at least one assay that detects LIFR mRNA. In certain embodiments, the level of LIFR is a level of LIFR DNA and determining the level comprises performing at least one assay that detects LIFR DNA or receiving the results of at least one assay that detects LIFR DNA. In certain embodiments, the at least one assay comprises polymerase chain reaction (PCR). In certain embodiments, the PCR comprises quantitative PCR. In certain embodiments, the at least one assay comprises a sequencing reaction. In certain embodiments, the sequencing reaction comprises a next-generation sequencing reaction. In certain embodiments, the biological sample comprises a blood sample. In certain embodiments, the blood sample is plasma. In certain embodiments, the blood sample is serum. In certain embodiments, the biological sample comprises a tissue sample. In certain embodiments, the biological sample is a tumor biopsy. In certain embodiments, the method further comprises determining a DNA, mRNA, or protein level of an immunomodulatory molecule that exceeds a reference level of the immunomodulatory molecule. In certain embodiments, the method further comprises determining a DNA, mRNA, or protein level of an immunomodulatory molecule that is below a reference level of the immunomodulatory molecule. In certain embodiments, the immunomodulatory molecule is selected from an mRNA transcribed from or a protein produced from the list consisting of MHCII, CXCL9, CXCL10, CXCR3, PD-L1, CCL7, CCL2, CCL3, and CCL22. In certain embodiments, the method further comprises determining a DNA, mRNA, or protein level of a Type II macrophage (M2) marker that exceeds a reference level of the Type II macrophage (M2) marker. In certain embodiments, the M2 marker is an mRNA transcribed from or a protein produced from the list consisting of CD206, CD163, PF4, CTSK, and ARG1. In certain embodiments, the method further comprises determining a DNA, mRNA, or protein level of LIF that exceeds a reference level of LIF. In certain embodiments, the human cancer is selected from the list consisting of non-small cell lung cancer, ovarian cancer, kidney cancer, bladder cancer, pancreatic cancer, prostate cancer, genitourinary cancer, gynecologic cancer, gastrointestinal cancer, endocrine system cancer, glioblastoma multiforme, breast cancer, melanoma, colorectal cancer, bile duct cancer, cervical cancer, endometrial cancer, head and neck squamous cell carcinoma, and combinations thereof. In certain embodiments, the human cancer is selected from the list consisting of non-small cell lung cancer, ovarian cancer, kidney cancer, bladder cancer, and combinations thereof. In certain embodiments, the human cancer is selected from the list consisting of pancreatic cancer, prostate cancer, glioblastoma multiforme, and combinations thereof.

In another aspect, described herein is a method of treating an individual with cancer with a therapeutic anti-Leukemia inhibitory factor (LIF) antibody comprising determining a level of Leukemia inhibitory factor receptor (LIFR) that exceeds a reference level in a biological sample from the individual, and administering a therapeutic amount of an the anti-LIF antibody to the individual. In certain embodiments, the level of LIFR is detected on an immunomodulatory cell. In certain embodiments, the therapeutic anti-LIF antibody comprises: an immunoglobulin heavy chain complementarity determining region 1 (VH-CDR1) comprising the amino acid sequence set forth in any one of SEQ ID NOs: 1-3; an immunoglobulin heavy chain complementarity determining region 2 (VH-CDR2) comprising the amino acid sequence set forth in any one of SEQ ID NOs: 4 or 5; an immunoglobulin heavy chain complementarity determining region 3 (VH-CDR3) comprising the amino acid sequence set forth in any one of SEQ ID NOs: 6-8; an immunoglobulin light chain complementarity determining region 1 (VL-CDR1) comprising the amino acid sequence set forth in any one of SEQ ID NOs: 9 or 10; an immunoglobulin light chain complementarity determining region 2 (VL-CDR2) comprising the amino acid sequence set forth in any one of SEQ ID NOs: 11 or 12; and an immunoglobulin light chain complementarity determining region 3 (VL-CDR3) comprising the amino acid sequence set forth in SEQ ID NO: 13. In certain embodiments, the therapeutic anti-LIF antibody comprises an immunoglobulin heavy chain variable region comprising at least 85%, 90%, 95%, 97%, 98%, 99%, or 100% identity to SEQ ID NOs: 14, 15, 17 or 38 and an immunoglobulin light chain variable region comprising at least 85%, 90%, 95%, 97%, 98%, 99%, or 100% identity to SEQ ID NO: 18-21. In certain embodiments, the therapeutic anti-LIF antibody comprises an immunoglobulin heavy chain region comprising at least 85%, 90%, 95%, 97%, 98%, 99%, or 100% identity to SEQ ID NOs: 30-33 or 39, and an immunoglobulin light chain region comprising at least 85%, 90%, 95%, 97%, 98%, 99%, or 100% identity to SEQ ID NOs: 34-37. In certain embodiments, the therapeutic anti-LIF antibody is an IgG antibody comprising two immunoglobulin heavy chains and two immunoglobulin light chains. In certain embodiments, the level of LIFR is a level of LIFR protein and determining the level comprises performing at least one assay that detects LIFR protein or receiving the results of at least one assay that detects LIFR protein. In certain embodiments, the at least one assay comprises immunohistochemistry. In certain embodiments, the at least one assay comprises enzyme linked immunosorbent assay (ELISA). In certain embodiments, the ELISA detects electrochemiluminescence. In certain embodiments, the at least one assay comprises flow cytometry. In certain embodiments, the level of LIFR is a level of LIFR mRNA and determining the level comprises performing at least one assay that detects LIFR mRNA or receiving the results of at least one assay that detects LIFR mRNA. In certain embodiments, the level of LIFR is a level of LIFR DNA and determining the level comprises performing at least one assay that detects LIFR DNA or receiving the results of at least one assay that detects LIFR DNA. In certain embodiments, the at least one assay comprises polymerase chain reaction (PCR). In certain embodiments, the PCR comprises quantitative PCR. In certain embodiments, the at least one assay comprises a sequencing reaction. In certain embodiments, the sequencing reaction comprises a next-generation sequencing reaction. In certain embodiments, the biological sample comprises a blood sample. In certain embodiments, the blood sample is plasma. In certain embodiments, the blood sample is serum. In certain embodiments, the biological sample comprises a tissue sample. In certain embodiments, the biological sample is a tumor biopsy. In certain embodiments, the method further comprises determining a DNA, mRNA, or protein level of an immunomodulatory molecule that exceeds a reference level of the immunomodulatory molecule. In certain embodiments, the method further comprises determining a DNA, mRNA, or protein level of an immunomodulatory molecule that is below a reference level of the immunomodulatory molecule. In certain embodiments, the immunomodulatory molecule is selected from an mRNA transcribed from or a protein produced from the list consisting of MHCII, CXCL9, CXCL10, CXCR3, PD-L1, CCL7, CCL2, CCL3, and CCL22. In certain embodiments, the method further comprises determining a DNA, mRNA, or protein level of a Type II macrophage (M2) marker that exceeds a reference level of the Type II macrophage (M2) marker. In certain embodiments, the M2 marker is an mRNA transcribed from or a protein produced from the list consisting of CD206, CD163, PF4, CTSK, and ARG1. In certain embodiments, the method further comprises determining a DNA, mRNA, or protein level of LIF that exceeds a reference level of LIF. In certain embodiments, the human cancer is selected from the list consisting of non-small cell lung cancer, ovarian cancer, kidney cancer, bladder cancer, pancreatic cancer, prostate cancer, genitourinary cancer, gynecologic cancer, gastrointestinal cancer, endocrine system cancer, glioblastoma multiforme, breast cancer, melanoma, colorectal cancer, bile duct cancer, cervical cancer, endometrial cancer, head and neck squamous cell carcinoma, and combinations thereof. In certain embodiments, the human cancer is selected from the list consisting of non-small cell lung cancer, ovarian cancer, kidney cancer, bladder cancer, and combinations thereof. In certain embodiments, the human cancer is selected from the list consisting of pancreatic cancer, prostate cancer, glioblastoma multiforme, and combinations thereof.

### BRIEF DESCRIPTION OF THE DRAWINGS

FIG. 1 depicts a western blot showing inhibition of LIF-induced STAT3 phosphorylation of different anti-LIF humanized antibodies.

FIGS. 2A and 2B depicts a western blot showing inhibition of LIF-induced STAT3 phosphorylation humanized and parental 5D8 antibody.

FIG. 3A shows an IC₅₀ for LIF inhibition in U-251 cells using the h5D8 antibody.

FIG. 3B shows representative IC₅₀ dose response curves of r5D8 and h5D8 inhibition of pSTAT3 under endogenous LIF stimulation conditions. Shown are the representative curves (n=1 h5D8, n=2 r5D8).

FIG. 4 depicts a western blot showing inhibition of LIF-induced STAT3 phosphorylation of different monoclonal antibodies described in this disclosure.

FIG. 5 depicts immunohistochemistry staining and quantitation of LIF expression in glioblastoma multiforme (GBM), NSCLC (non-small cell lung carcinoma), ovarian cancer, colorectal cancer tumors and pancreatic tumors from human patients. Bars represent mean+/−SEM.

FIG. 6 is a graph showing an experiment conducted in a mouse model of non-small cell lung cancer using the humanized 5D8 antibody.

FIG. 7A shows the effect of r5D8 on inhibition of U251 cells in an orthotopic mouse model of GBM. Quantitation shown at day 26.

FIG. 7B shows data from mice inoculated with luciferase expressing human U251 GBM cells and then treated with 100, 200 or 300 μg of h5D8 or vehicle twice a week. Tumor size was determined by bioluminescence (Xenogen IVIS Spectrum) on day 7. The graph shows individual tumor measurements with horizontal bars indicating mean±SEM. Statistical significance was calculated using the unpaired non-parametric Mann-Whitney U-test.

FIG. 8A shows the effect of r5D8 on inhibition of growth of ovarian cancer cells in an syngeneic mouse model.

FIG. 8B shows the individual measurements of tumors at day 25.

FIG. 8C illustrates that h5D8 shows a significant reduction in tumor growth when administered at 200 μg/mouse twice weekly (p<0.05). Symbols are mean+SEM, statistical significance compared with vehicle (with unpaired non-parametric Mann-Whitney U-test).

FIG. 9A shows the effect of r5D8 on inhibition of growth of colorectal cancer cells in a syngeneic mouse model.

FIG. 9B shows the individual measurements of tumors at day 17.

FIG. 10A shows reduction of macrophage infiltration to tumor sites in an orthotopic mouse model of GBM with a representative image and quantitation of CCL22+ cells.

FIG. 10B shows reduction of macrophage polarization in a human organotypic tissue slice culture model. Shown are a representative image (left) and quantitation (right).

FIG. 10C shows reduction of macrophage polarization to tumor sites in a syngeneic mouse model of ovarian cancer with a representative image and quantitation of CCL22+ cells.

FIG. 10D shows reduction of macrophage infiltration to tumor sites in a syngeneic mouse model of colorectal cancer with a representative image and quantitation of CCL22+ cells.

FIG. 11A shows increases in non-myeloid effector cells in a syngeneic mouse model of ovarian cancer after treatment with r5D8.

FIG. 11B shows increases in non-myeloid effector cells in a syngeneic mouse model of colorectal cancer after treatment with r5D8.

FIG. 11C shows decreases in percentage of CD4+ T_{REG} cells in a mouse model of NSCLC cancer after treatment with r5D8.

FIG. 12 shows data from mice bearing CT26 tumors treated twice weekly with PBS (control) or r5D8 administered intraperitoneally in the presence or absence of anti-CD4 and anti-CD8 depleting antibodies. The graph shows individual tumor measurements at d13 expressed as mean tumor volume+SEM. Statistical differences between groups was determined by unpaired non-parametric Mann-Whitney U-test. R5D8 inhibited the growth of CT26 tumors (*p<0.05). The tumor growth inhibition by r5D8 was significantly reduced in the presence of anti-CD4 and anti-CD8 depleting antibodies (****p<0.0001).

FIG. 13A illustrates an overview of the co-crystal structure of h5D8 Fab in complex with LIF. The gp130 interacting site is mapped on the surface of LIF (dark shaded).

FIG. 13B illustrates detailed interactions between LIF and h5D8, showing residues forming salt bridges and h5D8 residues with buried surface areas greater than 100 Å².

FIG. 14A illustrates superposition of the five h5D8 Fab crystal structures and indicates a high degree of similarity despite being crystallized in different chemical conditions.

FIG. 14B illustrates an extensive network of Van der Waals interactions mediated by unpaired Cys100. This residue is well-ordered, partakes in shaping the conformations of HCDR1 and HCDR3 and is not involved in undesired disulfide scrambling. Distances between residues are shown as dashed lines and labeled.

FIG. 15A illustrates binding of h5D8 C100 mutants to human LIF by ELISA.

FIG. 15B illustrates binding of h5D8 C100 mutants to mouse LIF by ELISA.

FIG. 16A illustrates that h5D8 does not block binding between LIF and LIFR by Octet. Sequential binding of h5D8 to LIF followed by LIFR.

FIGS. 16B and 16C illustrate ELISA analysis of LIF/mAb complexes binding to immobilized LIFR or gp130. Signals of species-specific peroxidase conjugated anti-IgG antibodies (anti-human for (−) and h5D8, anti-rat for r5d8 and B09) detecting the antibody portion of mAb/LIF complexes binding immobilized LIFR (FIG. 16B) or gp130 (FIG. 16C) coated plates.

FIGS. 17A and 17B illustrate mRNA expression of LIF (FIG. 16A) or LIFR (FIG. 16B) in 72 different human tissues.

FIG. 18 shows mRNA expression levels in different cancer types stratified into high, medium-high, medium low, and low levels. Expression data represents LIF transcript levels measured across 7,769 samples collected from 22 indications, collected from The Cancer Genome Atlas, and is thresholded by quartiles across the dataset.

FIG. 19A shows correlation (r²) of LIF mRNA levels with CCL7 mRNA levels. Samples for each indication were obtained from The Cancer Genome Atlas and the association between LIF and CCL7 was assessed by Pearson correlation.

FIG. 19B shows correlation (r²) of LIF mRNA levels with CCL2 mRNA levels. Samples for each indication were obtained from The Cancer Genome Atlas and the association between LIF and CCL2 was assessed by Pearson correlation.

FIG. 19C shows correlation (r²) of LIF mRNA levels with CCL3 mRNA levels. Samples for each indication were obtained from The Cancer Genome Atlas and the association between LIF and CCL3 was assessed by Pearson correlation.

FIG. 19D shows correlation (r²) of LIF mRNA levels with CCL22 mRNA levels. Samples for each indication were obtained from The Cancer Genome Atlas and the association between LIF and CCL22 was assessed by Pearson correlation.

FIG. 20A shows the correlation (r²) of LIF mRNA levels with an expression signature typical of Type II macrophage (M2).

FIG. 20B shows the correlation between LIF and CD163, CD206, and CCL2 expression in GBM and ovarian cancer. Regression plots are between LIF and CD163, CD206, CCL2 expression (in log 2 RSEM) in GBM and ovarian cancer (OV) TCGA tumor cohorts.

FIG. 20C shows correlation of IHC of the indicated markers from 20 GBM tumors. Correlations between LIF and CCL2, CD206, CD163, and CXCL9 with the R-squared coefficients (R²) are shown.

FIG. 20D shows percentage and mean fluorescent intensity (MFI) of CCL2⁺ and CXCL9⁺ in TAMs (CD11b⁺ Ly6G⁻ Ly6C⁻) from anti-LIF treated or untreated GL261N tumors.

FIG. 20E shows percentage of double positive cells relative to the TAM marker positive cells. CXCL9 quantification is relative to the total number of cells.

FIG. 20F shows percentage of CCR2, CXCR3 and LIFR receptors in TAMs (CD11b⁺ Ly6G⁻ Ly6C⁻) and CD8⁺ T cell (CD3⁺ CD8⁺) populations was determined by flow cytometry. Data are presented as mean±SEM. Statistical analysis by Mann-Whitney T test. *P<0.05.

FIG. 20G shows tumor growth of GL261N in CXCL9^{-/-} and CCL2^{-/-} mice or mice treated with the indicated antibodies is shown as total flux (p/s).

FIG. 20H shows fold increase (FI) of tumor infiltrating CD8⁺ T cells in the indicated treatments. Data are mean±SEM. Statistical analyses by Mann-Whitney T test. *P<0.05; **P<0.01; ***P<0.001; ****P<0.0001.

FIG. 20I shows percentage of double positive cells relative to Iba1⁺ cells and percentage of CXCL9⁺ cells in GBM organotypic slices (patients 1, 2, 3) incubated with 10 j·tg/ml anti-LIF for 3 days relative to the total number of cells. Data are mean of all patients±SEM. Statistical analyses by Mann-Whitney T test. *P<0.05, **P<0.01; ***P<0.001; ****P<0.0001.

FIG. 20J shows transcript levels of M1 and M2 genes in CT26 tumors at endpoint determined by qRT-PCR of whole tumor lysate (n=8). Data is represented as the fold-change relative to mean of IgG1 control treated tumors (Hprt, Tbp, Tfrc).

FIG. 20K shows the quantification of pan Myeloid cell population (CD11b⁺ in CD45⁺ cells), TAM (CD11b⁺ Ly6C^{low} F4/80⁺ in CD45⁺ cells), MHC TAMs (MHC II⁺ in CD11b⁺ Ly6C^{low} F4/80⁺ cells), and MHC II expression in TAMs. Data are shown as mean±s.e.m. (n is indicated for each experiment), *p<0.05, **p<0.01,***p<0.001.

FIGS. 21A and B show level of LIF receptor on primary macrophages differentiated from primary monocytes from 3 different donors by flow cytometry (FIG. 21A), and quantitated by interpolation against fluorescence calibration beads (FIG. 21B).

FIG. 22 shows increase in CD206 and CD163 in primary M₀ macrophages in response to LIF treatment (20 nM) for 72 hours.

FIG. 23 shows increase in CCL22 secretion in response to LIF treatment (20 nM) in M_{O} macrophages, and M1 and M2 macrophages (lower right corner).

FIG. 24 shows LIF receptor expression on tumor associated macrophages from Serous Ovarian Cancer, Stage III-C (two different donors, two left panels), and Lung Adenocarcinoma, stage III-A (two different donors, two right panels) determined by flow cytometry. Control plots are fluorescent minus one (FMO) controls.

FIG. 25 shows LIF receptor expression on tumor monocytic myeloid derived suppressor cells (M-MDSC) tumor polymorphonuclear myeloid derived suppressor cells (PMN-MDSC) from Serous Ovarian Cancer, Stage III-C (two different donors, two left panels), and Lung Adenocarcinoma, stage III-A (two different donors, two right panels) determined by flow cytometry. Control plots represent fluorescent minus one (FMO) staining. Samples are gated on CD11b⁺ CD33⁺ HLA-DR^{low}.

### DETAILED DESCRIPTION

In one aspect, described herein, is a method of treating an individual with cancer with a therapeutic anti-leukemia inhibitory factor (LIF) antibody comprising determining a level of LIF that exceeds a reference level in a biological sample from the individual, and administering a therapeutic amount of the anti-LIF antibody to the individual when the level of LIF is greater than the reference level of LIF.

In another aspect, described herein, is a method of treating an individual with cancer with a therapeutic anti-leukemia inhibitory factor (LIF) antibody comprising determining a level of LIF that exceeds a reference level in a biological sample from the individual, and administering a therapeutic amount of the anti-LIF antibody.

In another aspect, described herein, is a method of treating an individual with cancer with a therapeutic anti-Leukemia inhibitory factor (LIF) antibody comprising determining a level of Leukemia inhibitory factor receptor (LIFR) that exceeds a reference level in a biological sample from the individual, and administering a therapeutic amount of the anti-LIF antibody to the individual when the level of LIFR is greater than the reference level of LIFR.

In another aspect, described herein, is a method of treating an individual with cancer with a therapeutic anti-Leukemia inhibitory factor (LIF) antibody comprising determining a level of Leukemia inhibitory factor receptor (LIFR) that exceeds a reference level in a biological sample from the individual, and administering a therapeutic amount of an the anti-LIF antibody to the individual.

As used herein the term “individual” “patient” or “subject” refers to individuals diagnosed with, suspected of being afflicted with, or at-risk of developing a cancer, tumor or neoplasm. In certain embodiments, the individual is a mammal. In certain embodiments, the mammal is a mouse, rat, rabbit, dog, cat, horse, cow, sheep, pig, goat, llama, alpaca, or yak. In certain embodiments, the individual is a human.

As used herein, unless otherwise indicated, the term “immunomodulatory molecule” refers to any molecule, polypeptide, or protein, present in either a tumor or tumor-microenvironment that modulates or causes modulation of the innate and/or adaptive immune system, including but not limited to immunosuppressive chemokines, immunosuppressive cytokines, or checkpoint inhibitor molecules. Immunomodulatory molecules may be produced by immunomodulatory cells, tumor/cancer cells or stromal cells. Immunomodulatory molecules include by way of non-limiting example CCL7, CCL2, CCL3, CCL22, MHCII, CXCL9, CXCL10, CXCR3, and PD-L1.

As used herein, unless otherwise indicated, the term “immunomodulatory cell” refers to any cell of the immune system that has the ability to produce immunomodulatory factors and includes dendritic cells, macrophages, tumor-associated macrophages, type I macrophages, Type II macrophages, myeloid derived suppressor cells, tumor polymorphonuclear myeloid derived suppressor cells (PMN-MDSC), helper T cells, regulatory T cells, activated T cells, antigen experienced T cells, cytotoxic T cells, and the like.

As used herein, unless otherwise indicated, the term “antibody” includes antigen binding fragments of antibodies, i.e. antibody fragments that retain the ability to bind specifically to the antigen bound by the full-length antibody, e.g. fragments that retain one or more CDR regions. Examples of antibody fragments include, but are not limited to, Fab, Fab′, F(ab′)2, and Fv fragments; diabodies; linear antibodies; heavy chain antibodies, single-chain antibody molecules, e.g. single-chain variable region fragments (scFv), nanobodies and multispecific antibodies formed from antibody fragments with separate specificities, such as a bispecific antibody. In certain embodiments, the antibodies are humanized in such a way as to reduce an individual's immune response to the antibody. For example, the antibodies may be chimeric, e.g. non-human variable region with human constant region, or CDR grafted, e.g. non-human CDR regions with human constant region and variable region framework sequences. In certain embodiments, antibodies are deimmunized after humanization. Deimmunization involves removing or mutating one or more T-cell epitopes in the constant region of the antibody. In certain embodiments, the antibodies described herein are monoclonal. As used herein a “recombinant antibody” is an antibody that comprises an amino acid sequence derived from two different species or, or two different sources, and includes synthetic molecules, for example, an antibody that comprises a non-human CDR and a human framework or constant region. In certain embodiments, recombinant antibodies of the present invention are produced from a recombinant DNA molecule or synthesized. The terms “cancer” and “tumor” relate to the physiological condition in mammals characterized by deregulated cell growth. Cancer is a class of diseases in which a group of cells display uncontrolled growth or unwanted growth. Cancer cells can also spread to other locations, which can lead to the formation of metastases. Spreading of cancer cells in the body can, for example, occur via lymph or blood. Uncontrolled growth, intrusion, and metastasis formation are also termed malignant properties of cancers. These malignant properties differentiate cancers from benign tumors, which typically do not invade or metastasize.

As used herein a “therapeutic antibody” is one administered to an individual and intended to produce one or more beneficial effects useful in the treatment of cancer. Therapeutic antibodies of the current disclosure include antibodies that have CDR sequences, heavy and/or light chain immunoglobulin variable regions, or full immunoglobulin heavy and light chains identical to h5D8, or CDRs that vary from h5D8 but that possess similar binding characteristics (epitope, affinity, or biological effect) and can produce one or more beneficial effects useful to treat cancer.

As used herein a “therapeutic amount” is a dosage amount of a therapeutic antibody intended to produce one or more beneficial effects useful for treating cancer. Some specific therapeutic amounts are discussed in detail herein.

As used herein “treating” or “treatment” refers to the intervention in a disease state intended to produce one or more beneficial effects. For cancer/tumor purposes treatment includes methods that are intended to cause or do cause stable disease, partial response, complete response, extension of progression-free survival, extension of overall survival, tumor shrinkage, a delay in tumor growth, an arrest of tumor growth, or a prevention or reduction in metastasis. In certain cases the therapeutic methods described herein may be used as maintenance after successful treatment or to prevent recurrence or metastasis of a particular tumor or cancer. It is understood that not all individuals will respond to the same degree, or at all, to a given administration of therapeutic antibody, however even if no response is detected these individuals are nonetheless considered to have been treated.

As used herein a “biomarker” is a measurable molecule in an individual whose presence is indicative of a disease state of that individual. Without limitation biomarkers comprise, for example, proteins and their post-translational modifications, polypeptides, nucleic acids, DNAs, RNAs, amino acids, fatty acids, lipids, sterols, carbohydrates, or metabolites and metabolic intermediates of amino acids, fatty acids, lipids, sterols, and carbohydrates.

As used herein an “IHC-score” relates to a parameter used to quantify LIF or LIF receptor expression levels in a test sample. The IHC-score in a sample is determined by staining the sample with the anti-LIF or LIF receptor specific antibody using immunohistochemistry. Each tumor cell is given an intensity level ranging from 0 for no staining to 3+ for the most intense staining, 2+ is for the moderately staining cells and 1+ for weakly staining cells. An IHC score can then be calculated by the following equation:

[1×(% cells 1+)+2×(% cells 2+)+3×(% cells 3+)]

An IHC score can range from 1 to 300. The IHC-score in a sample can be used directly to provide an indication as to LIF expression levels or can be compared to a reference IHC-score value to provide an indication as to whether an individual would respond to treatment with an anti-LIF therapeutic antibody.

As used herein “immunohistochemistry” or “IHC” refers to a lab test that uses antibodies, affinity molecules and stains to test for certain antigens (biomarkers) in a sample of tissue or cells. The antibodies can be linked to an enzyme or a fluorescent dye. IHC can be combined with other non-antibody stains or methods that further elaborate tissue or cell structure, for example nuclear or cell membrane stains. IHC can be performed on formalin-fixed paraffin embedded or frozen tissue or biopsy samples. IHC can also be performed on cells in suspension with the cells subsequently being spun down or adhered to a microscope slide or cover slip. IHC samples can suitably be analyzed by visible light microscopy or imaging or fluorescent microscopy or imaging. Quantitation can be performed manually or by a computer program (e.g., Image J).

As used herein “reference level” relates to a predetermined criteria used as a reference for evaluating the values or data obtained from a sample obtained from an individual. The reference level can be an absolute value; a relative value; a value that has an upper or a lower limit; a range of values; an average value; a median value; a mean value; or a value as compared to a particular control or baseline value. A reference level can be based on an individual sample value, such as for example, a value obtained from a sample from the subject being tested, but at an earlier point in time. The reference level can be based on a large number of samples, such as from a population of subjects of similar chronological age, gender, disease state, or otherwise matched group, or based on a pool of samples including or excluding the sample to be tested. A reference level can also be determined from a representative number of cancer/tumor samples derived from different individuals afflicted with a cancer. A reference level can also be determined from biological samples from cancer or non-cancer afflicted individuals. These biological samples from a cancer afflicted or non-cancer afflicted individual may comprise for example, tissue biopsies, blood, plasma, serum, fecal samples, urine, cerebral spinal fluid, pap smears, or semen. A representative sample can include measurements from at least 50, 100, 200, 300, 400, 500, 600, 700, 800, 900, 1,000 or more individuals or cancer/tumor biological samples from individuals.

Percent (%) sequence identity with respect to a reference polypeptide or antibody sequence is the percentage of amino acid residues in a candidate sequence that are identical with the amino acid residues in the reference polypeptide or antibody sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN, or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “epitope” includes any determinant capable of being bound by an antigen binding protein, such as an antibody. An epitope is a region of an antigen that is bound by an antigen binding protein that targets that antigen, and when the antigen is a protein, includes specific amino acids that directly contact the antigen binding protein. Most often, epitopes reside on proteins, but in some instances can reside on other kinds of molecules, such as saccharides or lipids. Epitope determinants can include chemically active surface groupings of molecules such as amino acids, sugar side chains, phosphoryl or sulfonyl groups, and can have specific three dimensional structural characteristics, and/or specific charge characteristics. Generally, antibodies specific for a particular target antigen will preferentially recognize an epitope on the target antigen in a complex mixture of proteins and/or macromolecules. In certain embodiments, the antibodies disclosed herein recognize a linear sequence of amino acids. In certain embodiments, the antibodies disclosed herein recognize conformational (non-linear) arrays of amino acids.

### Structural Attributes of the Antibodies Described Herein

A complementarity determining region (“CDR”) is a part of an immunoglobulin (antibody) variable region that is primarily responsible for the antigen binding specificity of the antibody. CDR regions are highly variable from one antibody to the next even when the antibody specifically binds the same target or epitope. A heavy chain variable region comprises three CDR regions, abbreviated VH-CDR1, VH-CDR2, and VH-CDR3; and a light chain variable region comprises three CDR regions, abbreviated VL-CDR1, VL-CDR2, and VL-CDR3. These CDR regions are ordered consecutively in the variable region with the CDR1 being the most N-terminal and the CDR3 being the most C-terminal. Interspersed between the CDRs are framework regions which contribute to the structure and display much less variability than the CDR regions. A heavy chain variable region comprises four framework regions, abbreviated VH-FR1, VH-FR2, VH-FR3, and VH-FR4; and a light chain variable region comprises four framework regions, abbreviated VL-FR1, VL-FR2, VL-FR3, and VL-FR4. Complete full-sized bivalent antibodies comprising two heavy and light chains will comprise: 12 CDRs, with three unique heavy chain CDRs and three unique light chain CDRs; 16 FR regions, with four unique heavy chain FR regions and four unique light chain FR regions. In certain embodiments, the antibodies described herein minimally comprise three heavy chain CDRs. In certain embodiments, the antibodies described herein minimally comprise three light chain CDRs. In certain embodiments, the antibodies described herein minimally comprise three heavy chain CDRs and three light chain CDRs. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); and Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme). CDRs are identified herein from variable sequences provided using different numbering systems, herein with the Kabat, the IMGT, the Chothia numbering system, or any combination of the three. The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. In certain embodiments, CDRs defined from the variable regions disclosed herein comprise those defined according to the Chothia, Kabat, IMGT, Contact, or Aho method, or any combination thereof.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (See e.g., Portolano et al., J. Immunol. 150:880-887 (1993); Clarkson et al., Nature 352:624-628 (1991)). In certain embodiments, the antibodies described herein comprise variable regions of rat origin. In certain embodiments, the antibodies described herein comprise CDRs of rat origin. In certain embodiments, the antibodies described herein comprise variable regions of mouse origin. In certain embodiments, the antibodies described herein comprise CDRs of mouse origin.

Alterations (e.g., substitutions) may be made in CDRs, e.g., to improve antibody affinity. Such alterations may be made in CDR encoding codons with a high mutation rate during somatic maturation (See e.g., Chowdhury, Methods Mol. Biol. 207:179-196 (2008)), and the resulting variant can be tested for binding affinity. Affinity maturation (e.g., using error-prone PCR, chain shuffling, randomization of CDRs, or oligonucleotide-directed mutagenesis) can be used to improve antibody affinity (See e.g., Hoogenboom et al. in Methods in Molecular Biology 178:1-37 (2001)). CDR residues involved in antigen binding may be specifically identified, e.g., using alanine scanning mutagenesis or modeling (See e.g., Cunningham and Wells Science, 244:1081-1085 (1989)). CDR-H3 and CDR-L3 in particular are often targeted. Alternatively, or additionally, a crystal structure of an antigen-antibody complex is analyzed to identify contact points between the antibody and antigen. Such contact residues and neighboring residues may be targeted or eliminated as candidates for substitution. Variants may be screened to determine whether they contain the desired properties.

In certain embodiments, the antibodies described herein comprise a constant region in addition to a variable region. The heavy chain constant region (C_{H}) comprises four domains abbreviated C_{H}1, C_{H}2, C_{H}3, and C_{H}4, located at the C-terminal end of the full heavy chain polypeptide, C-terminal to the variable region. The light chain constant region (C_{L}) is much smaller than the C_{H} and is located at the C-terminal end of the full light chain polypeptide, C-terminal to the variable region. The constant region is highly conserved and comprises different isotypes that are associated with slightly different functions and properties. In certain embodiments, the constant region is dispensable for antibody binding to a target antigen. In certain embodiments, the constant regions of the antibody, both heavy and light chains are dispensable for antibody binding. In certain embodiments, the antibodies described herein lack one or more of a light chain constant region, heavy chain constant region, or both. Most monoclonal antibodies are of an IgG isotype; which is further divided into four subclasses IgG₁, IgG₂, IgG₃, and IgG₄. In certain embodiments, the antibodies described herein comprise any IgG subclass. In certain embodiments, the IgG subclass comprises IgG₁. In certain embodiments, the IgG subclass comprises IgG₂. In certain embodiments, the IgG subclass comprises IgG₃. In certain embodiments, the IgG subclass comprises IgG₄.

Antibodies comprise a fragment crystallizable region (Fc region) that is responsible for binding to complement and Fc receptors. The Fc region comprises the C_{H}2, C_{H}3, and C_{H}4 regions of the antibody molecule. The Fc region of an antibody is responsible for activating complement and antibody dependent cell cytotoxicity (ADCC). The Fc region also contributes to an antibody's serum half-life. In certain embodiments, the Fc region of the therapeutic antibodies described herein comprise one or more amino acid substitutions that promote complement mediated cell lysis. In certain embodiments, the Fc region of the therapeutic antibodies described herein comprises one or more amino acid substitutions that promote ADCC. In certain embodiments, the Fc region of the therapeutic antibodies described herein comprises one or more amino acid substitutions that reduce complement mediated cell lysis. In certain embodiments, the Fc region of the therapeutic antibodies described herein comprises one or more amino acid substitutions that increase binding of the antibody to an Fc receptor. In certain embodiments, the Fc receptor comprises FcγRI (CD64), FcγRIIA (CD32), FcγRIIIA (CD16a), FcγRIIIB (CD16b), or any combination thereof. In certain embodiments, the Fc region of the therapeutic antibodies described herein comprise one or more amino acid substitutions that increase the serum half-life of the antibody. In certain embodiments, the one or more amino acid substitutions that increase the serum half-life of the therapeutic antibody increase affinity of the antibody to the neonatal Fc receptor (FcRn).

Antibodies useful in the clinic are often “humanized” to reduce immunogenicity in human individuals. Humanized antibodies improve safety and efficacy of monoclonal antibody therapy. One common method of humanization is to produce a monoclonal antibody in any suitable animal (e.g., mouse, rat, hamster) and replace the constant region with a human constant region, antibodies engineered in this way are termed “chimeric”. Another common method is “CDR grafting” which replaces the non-human V-FRs with human V-FRs. In the CDR grafting method all residues except for the CDR region are of human origin. In certain embodiments, the antibodies described herein are humanized. In certain embodiments, the antibodies described herein are chimeric. In certain embodiments, the antibodies described herein are CDR grafted.

Methods of Determining Treatment with a Therapeutic Anti-LIF Antibody

Described herein are methods comprising treating an individual with a therapeutic anti-LIF antibody when the level of a biomarker exceeds a reference level in a sample from the individual. The sample can comprise a blood sample, plasma sample, serum sample, urine sample, fecal sample, or a tissue sample, such as a tissue biopsy from a suspected or known tumor. The biomarker can comprise LIF, LIF receptor, a marker of a type II macrophage (M2) cell, a marker of a regulatory T cell, an activated T cell, an antigen experienced T cell, a cytotoxic T cell, an immunosuppressive cytokine, or an immunosuppressive chemokine, or phosphorylated STAT3 or any other immunomodulatory molecule. Levels of biomarkers can be determined by any commonly used molecular or cellular technique, such as without limitation: mRNA quantitation, by semi-quantitative PCR, digital PCR, real-time PCR or RNA-seq; or protein quantitation, by western blot, flow cytometry, mass cytometry, ELISA, immunofluorescence, or a homogenous protein quantitation assays (e.g., AlphaLISA®). In certain embodiments, the biomarker is determined by immunohistochemistry using an antibody specific for a certain biomarker. Immunohistochemistry can be performed on a biopsy or a blood sample from the individual. Using immunohistochemistry an IHC-score for a certain protein can be determined and compared to a reference level or a control sample. Additionally, protein, mRNA, or DNA levels of combinations of biomarkers can be determined to inform treatment decisions.

In certain embodiments, the biomarker is LIF. In certain embodiments, an individual treated with the antibodies of this disclosure has been selected for treatment as having a LIF positive tumor/cancer. In certain embodiments, the tumor is LIF positive or produces elevated levels of LIF. In certain embodiments, LIF positivity is determined in comparison to a reference value or a set pathological criteria. In certain embodiments, a LIF positive tumor expresses greater than 2-fold, 3-fold, 5-fold, 10-fold, 100-fold or more LIF than a non-transformed cell from which the tumor is derived. In certain embodiments, the tumor has acquired ectopic expression of LIF.

LIF protein levels can be determined quantitatively or semi-quantitatively using immunohistochemistry. In certain embodiments, a LIF IHC-score can be calculated in a sample from an individual, and if the IHC score is or exceeds about 1, 5, 10, 25, 50, 75, 100, 110, 120, 130, 140, 150, 160, 170, 180, 190, 200, 210, 220, 230, 240, or 250, then a therapeutic anti-LIF antibody is administered to the individual. In certain embodiments, the therapeutic antibody is h5D8 or an antigen binding fragment thereof. In certain embodiments, the sample is a tissue sample or a tissue biopsy sample. In certain embodiments, a percentage of LIF positive cells can be determined in a sample from an individual, and if the percentage of LIF positive cells exceeds 1%, 2%, 3%. 4%, 5%, 6%, 7%, 8%, 9%, 10%, 15%, 20%, 25%, 30%, 35%, 40%, 45%, 50%, 55%, 60%, 65%, 70%, 75%, or 80%, then a therapeutic anti-LIF antibody is administered to the individual. In certain embodiments, the therapeutic antibody is h5D8 or an antigen binding fragment thereof. In certain embodiments, the sample is a tissue sample or a tissue biopsy sample. The LIF-IHC score reference level is derived from levels observed in a population of at least N samples. In certain embodiments, N is equal to or greater than 20, 30, 40, 50, 60, 70, 80, 90, 100, 150, 200, 250, 300, 350, 400, 450, 500 or more. In certain embodiments, the sample comprises cancers of like type (e.g., defining a reference level for a specific cancer) or all cancers (e.g., defining a reference level for all cancers). Different types of cancer may possess different reference levels that indicate an increased chance for successful treatment with h5D8, thus, a LIF IHC score may be specific to a certain cancer. In certain embodiments, the LIF IHC score is specific for any one or more of non-small cell lung cancer, ovarian cancer, kidney cancer, bladder cancer, pancreatic cancer, prostate cancer, genitourinary cancer, gynecologic cancer, gastrointestinal cancer, endocrine system cancer, glioblastoma multiforme, breast cancer, melanoma, colorectal cancer, bile duct cancer, cervical cancer, endometrial cancer, and head and neck squamous cell carcinoma.

LIF protein levels can be determined quantitatively or semi-quantitatively using an ELISA based assay. In certain embodiments, a LIF protein amount can be determined in a sample from an individual, and if the protein amount exceeds 1 picograms/milliliter (pg/mL), 2 pg/mL, 3 pg/mL, 4 pg/mL, 5 pg/mL, 6 pg/mL, 7 pg/mL, 8 pg/mL, 9 pg/mL, 10 pg/mL, 20 pg/mL, 30 pg/mL, 40 pg/mL, 50 pg/mL, 60 pg/mL, 70 pg/mL, 80 pg/mL, 90 pg/mL, then a therapeutic anti-LIF antibody is administered to the individual. In certain embodiments, the therapeutic antibody is h5D8 or an antigen binding fragment thereof. In certain embodiments, a LIF protein amount can be determined in sample from an individual, and if the protein amount exceeds 100 pg/mL), 200 pg/mL, 300 pg/mL, 400 pg/mL, 500 pg/mL, 600 pg/mL, 700 pg/mL, 800 pg/mL, 900 pg/mL, 1 nanograms/milliliter (ng/mL), 2 ng/mL, 3 ng/mL, 4 ng/mL, 5 ng/mL, 6 ng/mL, 7 ng/mL, 8 ng/mL, 9 ng/mL, 10 ng/mL, then a therapeutic anti-LIF antibody is administered to the individual. In certain embodiments, the therapeutic antibody is h5D8 or an antigen binding fragment thereof. In certain embodiments, a LIF protein amount can be determined in sample from an individual, and if the protein amount exceeds 100 picograms/milliliter (pg/mL), 200 pg/mL, 300 pg/mL, 400 pg/mL, 500 pg/mL, 600 pg/mL, 700 pg/mL, 800 pg/mL, 900 pg/mL, 1 nanograms/milliliter (ng/mL), 2 ng/mL, 3 ng/mL, 4 ng/mL, 5 ng/mL, 6 ng/mL, 7 ng/mL, 8 ng/mL, 9 ng/mL, 10 ng/mL, then a therapeutic anti-LIF antibody is administered to the individual. In certain embodiments, the therapeutic antibody is h5D8 or an antigen binding fragment thereof. In certain embodiments, the sample is a blood sample, plasma sample, or serum sample. The ELISA reference level is derived from levels observed in a population of at least N samples. In certain embodiments, N is equal to or greater than 20, 30, 40, 50, 60, 70, 80, 90, 100, 150, 200, 250, 300, 350, 400, 450, 500 or more. In certain embodiments, the sample comprises cancers of like type (e.g., defining a reference level for a specific cancer) or all cancers (e.g., defining a reference level for all cancers). Different types of cancer may possess different reference levels that indicate an increased chance for successful treatment with h5D8, thus, a LIF ELISA reference may be specific to a certain cancer. In certain embodiments, the LIF ELISA reference is specific for any one or more of non-small cell lung cancer, ovarian cancer, kidney cancer, bladder cancer, pancreatic cancer, prostate cancer, genitourinary cancer, gynecologic cancer, gastrointestinal cancer, endocrine system cancer, glioblastoma multiforme, breast cancer, melanoma, colorectal cancer, bile duct cancer, cervical cancer, endometrial cancer, and head and neck squamous cell carcinoma.

LIF mRNA levels can be determined quantitatively or semi-quantitatively using real-time PCR or RNA-seq. In certain embodiments, a LIF mRNA level can be determined and if the LIF mRNA level exceeds a level corresponding to the 25^{th}, 30^{th}, 35^{th}, 40^{th}, 45^{th}, 50^{th}, 55^{th}, 60^{th}, 65^{th}, 70^{th}, or 75^{th} percentile, then a therapeutic anti-LIF antibody is administered to the individual. In certain embodiments, the therapeutic antibody is h5D8 or an antigen binding fragment thereof. The percentile reference relates to mRNA levels observed in a population of at least N samples. In certain embodiments, N is equal to or greater than 20, 30, 40, 50, 60, 70, 80, 90, 100, 150, 200, 250, 300, 350, 400, 450, 500 or more. In certain embodiments, the sample comprises cancers of like type (e.g., defining a reference level for a specific cancer) or all cancers (e.g., defining a reference level for all cancers). Different types of cancer may possess different reference levels that indicate an increased chance for successful treatment with h5D8, thus, a LIF mRNA reference level may be specific to a certain cancer. In certain embodiments, the LIF mRNA reference level is specific for any one or more of non-small cell lung cancer, ovarian cancer, kidney cancer, bladder cancer, pancreatic cancer, prostate cancer, genitourinary cancer, gynecologic cancer, gastrointestinal cancer, endocrine system cancer, glioblastoma multiforme, breast cancer, melanoma, colorectal cancer, bile duct cancer, cervical cancer, endometrial cancer, and head and neck squamous cell carcinoma.

LIF signals through binding to the LIF receptor and gp130. In certain embodiments, the antibodies disclosed herein, are useful for treating tumors or cancers that express the LIF receptor (CD118), either directly on the cancer cell, or on tumor associated myeloid cells (e.g., macrophages or myeloid derived suppressor cells), stromal cells (cancer associated fibroblasts), or endothelial cells. The tumor associated macrophages can be specific immunosuppressive macrophages such as type II macrophages (M2).

In certain embodiments, the biomarker is LIF receptor. In certain embodiments, an individual treated with the antibodies of this disclosure has been selected for treatment as having a LIF receptor positive tumor/cancer. In certain embodiments, an individual treated with the antibodies of this disclosure has been selected for treatment as having LIF receptor positive infiltrates to tumor sites, as assessed by, for example, IHC, flow cytometry, or mRNA quantitation. These infiltrates can comprise immunomodulatory cells such as tumor associated macrophages, type II macrophages, myeloid derived suppressor cells, tumor monocytic myeloid derived suppressor cells (M-MDSC), or tumor polymorphonuclear myeloid derived suppressor cells (PMN-MDSC).

In certain embodiments, the antibodies disclosed herein, are useful for treating tumors or cancers that express the LIF receptor. A LIF receptor positive tumor can be determined by histopathology or flow cytometry, and, in certain embodiments, comprises a cell that binds a LIF receptor antibody greater than 2×, 3×, 4×, 5×, 10× or more than an isotype control. In certain embodiments, the tumor has acquired ectopic expression of the LIF receptor. In a certain embodiment, the cancer cell is a cancer stem cell. In a certain embodiment, a LIF receptor positive tumor or cancer can be determined by immunohistochemistry using anti-LIF receptor. In certain embodiments, a level of LIF receptor protein or mRNA is determined associated with one or more cell populations associated with an immunosuppressive response. In certain embodiments, the cell population is myeloid cells, macrophage cells, M2 cells, neutrophils, myeloid derived suppressor cells, tumor M-MDSC, or tumor PMN-MDSC.

LIF receptor protein levels can be determined quantitatively or semi-quantitatively using immunohistochemistry. An IHC assay for LIF receptor can be based upon LIF receptor expressed on all cells in a sample, on all immune cells in a sample, all myeloid derived cells in a sample, or all macrophages in a sample. In certain embodiments, a LIF receptor IHC-score can be calculated in sample from an individual, and if the IHC score is or exceeds about 1, 5, 10, 25, 50, 75, 100, 110, 120, 130, 140, 150, 160, 170, 180, 190, 200, 210, 220, 230, 240, or 250, then a therapeutic anti-LIF antibody is administered to the individual. In certain embodiments, the therapeutic antibody is h5D8 or an antigen binding fragment thereof. In certain embodiments, the sample is a tissue sample or a tissue biopsy sample. In certain embodiments, a percentage of LIF receptor positive cells can be determined in a sample from an individual, and if the percentage of LIF receptor positive cells exceeds 1%, 2%, 3%. 4%, 5%, 6%, 7%, 8%, 9%, 10%, 15%, 20%, 25%, 30%, 35%, 40%, 45%, 50%, 55%, 60%, 65%, 70%, 75%, or 80%, then a therapeutic anti-LIF antibody is administered to the individual. In certain embodiments, the therapeutic antibody is h5D8 or an antigen binding fragment thereof. In certain embodiments, the sample is a tissue sample or a tissue biopsy sample. The LIF receptor IHC score reference level is derived from levels observed in a population of at least N samples. In certain embodiments, N is equal to or greater than 20, 30, 40, 50, 60, 70, 80, 90, 100, 150, 200, 250, 300, 350, 400, 450, 500 or more. In certain embodiments, the sample comprises cancers of like type (e.g., defining a reference level for a specific cancer) or all cancers (e.g., defining a reference level for all cancers). Different types of cancer may possess different reference levels that indicate an increased chance for successful treatment with h5D8, thus, a LIF receptor IHC score may be specific to a certain cancer. In certain embodiments, the LIF receptor IHC score is specific for any one or more of non-small cell lung cancer, ovarian cancer, kidney cancer, bladder cancer, pancreatic cancer, prostate cancer, genitourinary cancer, gynecologic cancer, gastrointestinal cancer, endocrine system cancer, glioblastoma multiforme, breast cancer, melanoma, colorectal cancer, bile duct cancer, cervical cancer, endometrial cancer, and head and neck squamous cell carcinoma.

LIF receptor protein levels can be determined quantitatively or semi-quantitatively using a flow cytometry based assay. In certain embodiments, a LIF receptor protein level can be determined in sample from an individual, and if the protein amount exceeds 1.5×, 2×, 3×, 4×, 5×, 6×, 7×, 8×, 9×, 10× compared to a control antibody (e.g., isotype control), then a therapeutic anti-LIF antibody is administered to the individual. In certain embodiments, the therapeutic antibody is h5D8 or an antigen binding fragment thereof. In certain embodiments, the sample is a blood sample, plasma sample, or serum sample. The flow cytometry reference level is derived from levels observed in a population of at least N samples. In certain embodiments, N is equal to or greater than 20, 30, 40, 50, 60, 70, 80, 90, 100, 150, 200, 250, 300, 350, 400, 450, 500 or more. In certain embodiments, the sample comprises cancers of like type (e.g., defining a reference level for a specific cancer) or all cancers (e.g., defining a reference level for all cancers). Different types of cancer may possess different reference levels that indicate an increased chance for successful treatment with h5D8, thus, a LIF flow cytometry reference score may be specific to a certain cancer. In certain embodiments, the LIF flow cytometry reference is specific for any one or more of non-small cell lung cancer, ovarian cancer, kidney cancer, bladder cancer, pancreatic cancer, prostate cancer, genitourinary cancer, gynecologic cancer, gastrointestinal cancer, endocrine system cancer, glioblastoma multiforme, breast cancer, melanoma, colorectal cancer, bile duct cancer, cervical cancer, endometrial cancer, and head and neck squamous cell carcinoma.

LIF receptor mRNA levels can be determined quantitatively or semi-quantitatively using real-time PCR or RNA-seq. In certain embodiments, a LIF receptor mRNA level can be determined and if the LIF receptor mRNA level exceeds a level corresponding to the 25^{th}, 30^{th}, 35^{th}, 40^{th}, 45^{th}, 50^{th}, 55^{th}, 60^{th}, 65^{th}, 70^{th}, or 75^{th} percentile, then a therapeutic anti-LIF antibody is administered to the individual. In certain embodiments, the therapeutic antibody is h5D8 or an antigen binding fragment thereof. The percentile reference relates to mRNA levels observed in a population of at least N samples. In certain embodiments, N is equal to or greater than 20, 30, 40, 50, 60, 70, 80, 90, 100, 150, 200, 250, 300, 350, 400, 450, 500 or more. In certain embodiments, the sample comprises cancers of like type (e.g., defining a reference level for a specific cancer) or all cancers (e.g., defining a reference level for all cancers). Different types of cancer may possess different reference levels that indicate an increased chance for successful treatment with h5D8, thus, a LIF mRNA reference level may be specific to a certain cancer. In certain embodiments, the LIF mRNA reference level is specific for any one or more of non-small cell lung cancer, ovarian cancer, kidney cancer, bladder cancer, pancreatic cancer, prostate cancer, genitourinary cancer, gynecologic cancer, gastrointestinal cancer, endocrine system cancer, glioblastoma multiforme, breast cancer, melanoma, colorectal cancer, bile duct cancer, cervical cancer, endometrial cancer, and head and neck squamous cell carcinoma.

Additional biomarkers described herein and useful in methods of treating an individual with a therapeutic anti-LIF antibody include immunosuppressive biomarkers. It is shown herein that LIF and LIF receptor are important for signaling in various immunomodulatory cells types, and thus, immunosuppressive biomarkers can serve as indicators of potential treatment success. These biomarkers can be utilized on their own, or combined with a determination of LIF and LIF receptor levels. In certain embodiments, if a protein, mRNA, or DNA level of an immunosuppressive biomarker exceeds a reference level, then a therapeutic anti-LIF antibody is administered to the individual. In certain embodiments, the therapeutic antibody is h5D8 or an antigen binding fragment thereof. In certain embodiments, if a protein, mRNA, or DNA level of an immunosuppressive biomarker exceeds a reference level and LIF exceeds a reference level, then a therapeutic anti-LIF antibody is administered to the individual. In certain embodiments, the therapeutic antibody is h5D8 or an antigen binding fragment thereof. In certain embodiments, if a protein, mRNA, or DNA level of an immunosuppressive biomarker exceeds a reference level and LIF receptor exceeds a reference level, then a therapeutic anti-LIF antibody is administered to the individual. In certain embodiments, the therapeutic antibody is h5D8 or an antigen binding fragment thereof.

In certain embodiments, a combination of all three of LIF, LIF receptor and an immunosuppressive biomarker can be utilized to select an individual for treatment. Important immunomodulatory and immunosuppressive biomarkers of the current disclosure include those that are associated with regulatory T cells, activated T cells, antigen experienced T cells, cytotoxic T cells, and their respective functions, including chemokines and cytokines released by tumor associated macrophages or present in the tumor micro environment; markers of myeloid derived suppressor cells, or markers of macrophages, including M2 macrophages. In certain embodiments, the biomarker is an immunomodulatory molecule, such as a costimulatory molecule, antigen presenting molecule, cytokine or chemokine that acts upon T regulatory cells. In certain embodiments, the costimulatory molecule, antigen presenting molecule, cytokine, or chemokine that acts upon T regulatory cells is selected from the list consisting of MHCII, CXCL9, CXCL10, CXCR3, PD-L1, CCL7, CCL2, CCL3, and CCL22. In certain embodiments, the antigen presenting molecule that acts upon T regulatory cells is MHCII. In certain embodiments, the cytokine or chemokine that acts upon T regulatory cells is CXCL9. In certain embodiments, the cytokine or chemokine that acts upon T regulatory cells is CXCL10. In certain embodiments, the cytokine or chemokine that acts upon T regulatory cells is CXCR3. In certain embodiments, the costimulatory molecule that acts upon T regulatory cells is PD-L1. In certain embodiments, the cytokine or chemokine that acts upon T regulatory cells is CCL7. In certain embodiments, the cytokine or chemokine that acts upon T regulatory cells is CCL2. In certain embodiments, the cytokine or chemokine that acts upon T regulatory cells is CCL3. In certain embodiments, the cytokine or chemokine that acts upon T regulatory cells is CCL22. In certain embodiments, a patient is selected for treatment if levels of MHCII are below a reference level. In certain embodiments, if MHCII is below 1.5×, 2×, 3×, 4×, 5×, 6×, 7×, 8×, 9×, 10× compared to a control antibody (e.g., isotype control), then a therapeutic anti-LIF antibody is administered to the individual. In certain embodiments, a patient is selected for treatment if levels of CXCL9 are below a reference level. In certain embodiments, if CXCL9 is below 1.5×, 2×, 3×, 4×, 5×, 6×, 7×, 8×, 9×, 10× compared to a control antibody (e.g., isotype control), then a therapeutic anti-LIF antibody is administered to the individual. In certain embodiments, a patient is selected for treatment if levels of CXCL10 are below a reference level. In certain embodiments, if CXCL10 is below 1.5×, 2×, 3×, 4×, 5×, 6×, 7×, 8×, 9×, 10× compared to a control antibody (e.g., isotype control), then a therapeutic anti-LIF antibody is administered to the individual. In certain embodiments, a patient is selected for treatment if levels of CXCR3 are below a reference level. In certain embodiments, if CXCR3 is below 1.5×, 2×, 3×, 4×, 5×, 6×, 7×, 8×, 9×, 10× compared to a control antibody (e.g., isotype control), then a therapeutic anti-LIF antibody is administered to the individual. In certain embodiments, a patient is selected for treatment if levels of PD-L1 are below a reference level. In certain embodiments, if PD-L1 is below 1.5×, 2×, 3×, 4×, 5×, 6×, 7×, 8×, 9×, 10× compared to a control antibody (e.g., isotype control), then a therapeutic anti-LIF antibody is administered to the individual. In certain embodiments, a patient is selected for treatment if levels of CCL7 exceed a reference level. In certain embodiments, if CCL7 exceeds 1.5×, 2×, 3×, 4×, 5×, 6×, 7×, 8×, 9×, 10× compared to a control antibody (e.g., isotype control), then a therapeutic anti-LIF antibody is administered to the individual. In certain embodiments, a patient is selected for treatment if levels of CCL2 exceed a reference level. In certain embodiments, if CCL2 exceeds 1.5×, 2×, 3×, 4×, 5×, 6×, 7×, 8×, 9×, 10× compared to a control antibody (e.g., isotype control), then a therapeutic anti-LIF antibody is administered to the individual. In certain embodiments, a patient is selected for treatment if levels of CCL3 exceed a reference level. In certain embodiments, if CCL3 exceeds 1.5×, 2×, 3×, 4×, 5×, 6×, 7×, 8×, 9×, 10× compared to a control antibody (e.g., isotype control), then a therapeutic anti-LIF antibody is administered to the individual. In certain embodiments, a patient is selected for treatment if levels of CCL22 exceed a reference level. In certain embodiments, if CCL22 exceeds 1.5×, 2×, 3×, 4×, 5×, 6×, 7×, 8×, 9×, 10× compared to a control antibody (e.g., isotype control), then a therapeutic anti-LIF antibody is administered to the individual. In certain embodiments, the immunosuppressive biomarker is a marker of M2 macrophage cells. In certain embodiments, the marker of M2 macrophage cells is selected from the list consisting of CD206, CD163, PF4, CTSK, and ARG1. In certain embodiments, the marker of M2 macrophage cells is CD206. In certain embodiments, the marker of M2 macrophage cells is CD163. In certain embodiments, the marker of M2 macrophage cells is PF4. In certain embodiments, the marker of M2 macrophage cells is CTSK. In certain embodiments, the marker of M2 macrophage cells is ARG1. In certain embodiments, a patient is selected for treatment if levels of CD206 exceed a reference level. In certain embodiments, if CD206 exceeds 1.5×, 2×, 3×, 4×, 5×, 6×, 7×, 8×, 9×, 10× compared to a control antibody (e.g., isotype control), then a therapeutic anti-LIF antibody is administered to the individual. In certain embodiments, a patient is selected for treatment if levels of CD163 exceed a reference level. In certain embodiments, if CD163 exceeds 1.5×, 2×, 3×, 4×, 5×, 6×, 7×, 8×, 9×, 10× compared to a control antibody (e.g., isotype control), then a therapeutic anti-LIF antibody is administered to the individual. In certain embodiments, a patient is selected for treatment if levels of PF4 exceed a reference level. In certain embodiments, if PF4 exceeds 1.5×, 2×, 3×, 4×, 5×, 6×, 7×, 8×, 9×, 10× compared to a control antibody (e.g., isotype control), then a therapeutic anti-LIF antibody is administered to the individual. In certain embodiments, a patient is selected for treatment if levels of CTSK exceed a reference level. In certain embodiments, if CTSK exceeds 1.5×, 2×, 3×, 4×, 5×, 6×, 7×, 8×, 9×, 10× compared to a control antibody (e.g., isotype control), then a therapeutic anti-LIF antibody is administered to the individual. In certain embodiments, a patient is selected for treatment if levels of ARG1 exceed a reference level. In certain embodiments, if ARG1 exceeds 1.5×, 2×, 3×, 4×, 5×, 6×, 7×, 8×, 9×, 10× compared to a control antibody (e.g., isotype control), then a therapeutic anti-LIF antibody is administered to the individual. Protein levels of immunosuppressive biomarkers can be determined by western blot, ELISA, flow cytometry or IHC; mRNA levels of immunosuppressive biomarkers can be determined by quantitative PCR or RNA-seq. In certain embodiments, the therapeutic antibody is h5D8 or an antigen binding fragment thereof. In certain embodiments, if a level of an immunosuppressive biomarker described herein exceeds a reference level, then a patient is selected for treatment with a therapeutic anti-LIF antibody. In certain embodiments, if a level of an immunosuppressive biomarker exceeds a reference level and LIF exceeds a reference level, then a therapeutic anti-LIF antibody is administered to the individual. In certain embodiments, if a level of an immunosuppressive biomarker exceeds a reference level and LIF receptor exceeds a reference level, then a therapeutic anti-LIF antibody is administered to the individual. In certain embodiments, a combination of all three of LIF, LIF receptor and an immunosuppressive biomarker can be utilized to select an individual for treatment.

Additional biomarkers described herein and useful in methods of determining treatment an individual with a therapeutic anti-LIF antibody include markers of LIF signaling. In certain embodiments, the marker of LIF signaling is phosphorylated STAT3. In certain embodiments, a patient is selected for treatment if levels of phosphorylated STAT3 exceed a reference level. In certain embodiments, if pSTAT3 exceeds 1.5×, 2×, 3×, 4×, 5×, 6×, 7×, 8×, 9×, 10× compared to a control antibody (e.g., isotype control), then a therapeutic anti-LIF antibody is administered to the individual. In certain embodiments, the therapeutic antibody is h5D8 or an antigen binding fragment thereof.

### Therapeutic Anti-LIF Antibodies

The 5D8 antibody described herein was generated from rats immunized with DNA encoding human LIF. The parental rat version of the antibody is referred to as r5D8 the humanized version is referred to as h5D8.

### 5D8

The antibodies described herein were generated from rats immunized with DNA encoding human LIF. One such antibody (5D8) was cloned and sequenced and comprises CDRs (using the combination of the Kabat and IMGT CDR numbering methods) with the following amino acid sequences: a VH-CDR1 corresponding to SEQ ID NO: 1 (GFTFSHAWMH), a VH-CDR2 corresponding to SEQ ID NO: 4 (QIKAKSDDYATYYAESVKG), a VH-CDR3 corresponding to SEQ ID NO: 6 (TCWEWDLDF), a VL-CDR1 corresponding to SEQ ID NO: 9 (RSSQSLLDSDGHTYLN), a VL-CDR2 corresponding to SEQ ID NO: 11 (SVSNLES), and a VL-CDR3 corresponding to SEQ ID NO: 13 (MQATHAPPYT). This antibody has been humanized by CDR grafting and the humanized version is referred to as h5D8. The V_{H} and V_{L} regions are set forth in SEQ ID NOs: 15 and 19.

In certain embodiments, described herein, is a therapeutic antibody that specifically binds LIF comprising a VH-CDR1 at least 80% or 90% identical to that set forth in SEQ ID NO: 1 (GFTFSHAWMH), a VH-CDR2 at least 80%, 90%, or 95% identical to that set forth in SEQ ID NO: 4 (QIKAKSDDYATYYAESVKG), and a VH-CDR3 at least 80% or 90% identical to that set forth in SEQ ID NO: 6 (TCWEWDLDF). In certain embodiments, described herein, is a therapeutic antibody that specifically binds LIF comprising a VL-CDR1 at least 80% or 90% identical to that set forth in SEQ ID NO: 9 (RSSQSLLDSDGHTYLN), a VL-CDR2 at least 80% identical to that set forth in SEQ ID NO: 11 (SVSNLES), and a VL-CDR3 at least 80% or 90% identical to that set forth in SEQ ID NO: 13 (MQATHAPPYT). In certain embodiments, described herein, is a therapeutic antibody that specifically binds LIF comprising a VH-CDR1 set forth in SEQ ID NO: 1 (GFTFSHAWMH), a VH-CDR2 set forth in SEQ ID NO: 4 (QIKAKSDDYATYYAESVKG), a VH-CDR3 set forth in SEQ ID NO: 6 (TCWEWDLDF), a VL-CDR1 set forth in SEQ ID NO: 9 (RSSQSLLDSDGHTYLN), a VL-CDR2 set forth in SEQ ID NO: 11 (SVSNLES), and a VL-CDR3 set forth in SEQ ID NO: 13 (MQATHAPPYT). Certain conservative amino acid substitutions are envisioned in the amino acid sequences of the CDRs of this disclosure. In certain embodiments, the antibody comprises CDRs that differ from the amino acid sequence set forth in any one of SEQ ID NOs: 1, 4, 6, 9, 11, and 13 by 1, 2, 3, or 4 amino acids. In certain embodiments, the antibody comprises CDRs that differ from the amino acid sequence set forth in any one of SEQ ID NOs: 1, 4, 6, 9, 11, and 13 by 1, 2, 3, or 4 amino acids and does not affect the binding affinity by greater than 10%, 20%, or 30%. In certain embodiments, antibodies that specifically bind LIF comprise one or more human heavy chain framework regions.

In certain embodiments, described herein, is a therapeutic antibody that specifically binds LIF comprising a VH-CDR1 amino acid sequence at least 80% or 90% identical to that set forth in SEQ ID NO: 1 (GFTFSHAWMH), a VH-CDR2 amino acid sequence at least 80%, 90%, or 95% identical to that set forth in SEQ ID NO: 4 (QIKAKSDDYATYYAESVKG), and a VH-CDR3 amino acid sequence at least 80% or 90% identical to that set forth in SEQ ID NO: 8 (TSWEWDLDF). In certain embodiments, described herein, is a therapeutic antibody that specifically binds LIF comprising a VL-CDR1 amino acid sequence at least 80% or 90% identical to that set forth in SEQ ID NO: 9 (RSSQSLLDSDGHTYLN), a VL-CDR2 amino acid sequence at least 80% identical to that set forth in SEQ ID NO: 11 (SVSNLES), and a VL-CDR3 amino acid sequence at least 80% or 90% identical to that set forth in SEQ ID NO: 13 (MQATHAPPYT). In certain embodiments, described herein, is a therapeutic antibody that specifically binds LIF comprising a VH-CDR1 amino acid sequence set forth in SEQ ID NO: 1 (GFTFSHAWMH), a VH-CDR2 amino acid sequence set forth in SEQ ID NO: 4 (QIKAKSDDYATYYAESVKG), a VH-CDR3 amino acid sequence set forth in SEQ ID NO: 8 (TSWEWDLDF), a VL-CDR1 amino acid sequence set forth in SEQ ID NO: 9 (RSSQSLLDSDGHTYLN), a VL-CDR2 amino acid sequence set forth in SEQ ID NO: 11 (SVSNLES), and a VL-CDR3 amino acid sequence set forth in SEQ ID NO: 13 (MQATHAPPYT). Certain conservative amino acid substitutions are envisioned in the amino acid sequences of the CDRs of this disclosure. In certain embodiments, the antibody comprises CDRs that differ from the amino acid sequence set forth in any one of SEQ ID NOs: 1, 4, 8, 9, 11, and 13 by 1, 2, 3, or 4 amino acids. In certain embodiments, the antibody comprises CDRs that differ from the amino acid sequence set forth in any one of SEQ ID NOs: 1, 4, 8, 9, 11, and 13 by 1, 2, 3, or 4 amino acids and does not affect the binding affinity by greater than 10%, 20%, or 30%.

In certain embodiments, described herein, is a therapeutic antibody that specifically binds LIF comprising a humanized heavy chain variable region comprising an amino acid sequence at least about 80%, about 90%, about 95%, about 97%, about 98%, or about 99% identical to the amino acid sequence set forth in any one of SEQ ID NOs: 14, 15, or 17. In certain embodiments, described herein, is a therapeutic antibody that specifically binds LIF comprising a humanized heavy chain variable region comprising an amino acid sequence set forth in any one of SEQ ID NOs: 14, 15, and 17. In certain embodiments, described herein, is a therapeutic antibody that specifically binds LIF comprising a humanized light chain variable region comprising an amino acid sequence at least about 80%, about 90%, about 95%, about 97%, about 98%, or about 99% identical to the amino acid sequence set forth in any one of SEQ ID NOs: 18-21. In certain embodiments, described herein, is a therapeutic antibody that specifically binds LIF comprising a humanized light chain variable region comprising an amino acid sequence set forth in any one of SEQ ID NOs: 18-21. In certain embodiments, the antibody specifically binds human LIF.

In certain embodiments, described herein, is a therapeutic antibody that specifically binds LIF comprising a humanized heavy chain variable region comprising an amino acid sequence at least about 80%, about 90%, about 95%, about 97%, about 98%, or about 99% identical to the amino acid sequence set forth in SEQ ID NO:15; and a humanized light chain variable region comprising an amino acid sequence at least about 80%, about 90%, about 95%, about 97%, about 98%, or about 99% identical to the amino acid sequence set forth in SEQ ID NO: 19. In certain embodiments, described herein, is a therapeutic antibody that specifically binds LIF comprising a humanized heavy chain variable region comprising an amino acid sequence set forth in SEQ ID NO: 15; and a humanized light chain variable region comprising an amino acid sequence set forth in SEQ ID NO: 19.

In certain embodiments, described herein, is a therapeutic antibody that specifically binds LIF comprising a humanized heavy chain variable region comprising an amino acid sequence at least about 80%, about 90%, about 95%, about 97%, about 98%, or about 99% identical to the amino acid sequence set forth in SEQ ID NO: 38; and a humanized light chain variable region comprising an amino acid sequence at least about 80%, about 90%, about 95%, about 97%, about 98%, or about 99% identical to the amino acid sequence set forth in SEQ ID NO: 19. In certain embodiments, described herein, is a therapeutic antibody that specifically binds LIF comprising a humanized heavy chain variable region comprising an amino acid sequence set forth in SEQ ID NO: 38; and a humanized light chain variable region comprising an amino acid sequence set forth in SEQ ID NO: 19.

In certain embodiments, described herein, is a therapeutic antibody that specifically binds LIF comprising a humanized heavy chain comprising an amino acid sequence at least about 80%, about 90%, about 95%, about 97%, about 98%, or about 99% identical to the amino acid sequence set forth in any one of SEQ ID NOs: 30-33; and a humanized light chain comprising an amino acid sequence at least about 80%, about 90%, about 95%, about 97%, about 98%, or about 99% identical to the amino acid sequence set forth in any one of SEQ ID NOs: 34-37. In certain embodiments, described herein, is a therapeutic antibody that specifically binds LIF comprising a humanized heavy chain comprising an amino acid sequence set forth in any one of SEQ ID NOs: 30-33; and a humanized light chain comprising an amino acid sequence set forth in any one of SEQ ID NOs: 34-37.

In certain embodiments, described herein, is a therapeutic antibody that specifically binds LIF comprising a humanized heavy chain comprising an amino acid sequence at least about 80%, about 90%, about 95%, about 97%, about 98%, or about 99% identical to the amino acid sequence set forth in SEQ ID NO: 31; and a humanized light chain comprising an amino acid sequence at least about 80%, about 90%, about 95%, about 97%, about 98%, or about 99% identical to the amino acid sequence set forth in SEQ ID NO: 35. In certain embodiments, described herein, is a therapeutic antibody that specifically binds LIF comprising a humanized heavy chain comprising an amino acid sequence set forth in SEQ ID NO: 31; and a humanized light chain comprising an amino acid sequence set forth in SEQ ID NO: 35. In certain embodiments, described herein, is a therapeutic antibody that specifically binds LIF comprising a humanized heavy chain comprising an amino acid sequence at least about 80%, about 90%, about 95%, about 97%, about 98%, or about 99% identical to the amino acid sequence set forth in SEQ ID NO:39; and a humanized light chain comprising an amino acid sequence at least about 80%, about 90%, about 95%, about 97%, about 98%, or about 99% identical to the amino acid sequence set forth in SEQ ID NO: 35. In certain embodiments, described herein, is a therapeutic antibody that specifically binds LIF comprising a humanized heavy chain comprising an amino acid sequence set forth in SEQ ID NO: 39; and a humanized light chain comprising an amino acid sequence set forth in SEQ ID NO: 35.

In certain embodiments, described herein, is a recombinant antibody that specifically binds Leukemia Inhibitory Factor (LIF) comprising: a heavy chain complementarity determining region 1 (VH-CDR1) comprising an amino acid sequence set forth in SEQ ID NO: 3; a heavy chain complementarity determining region 2 (VH-CDR2) comprising an amino acid sequence set forth in SEQ ID NO: 4; a heavy chain complementarity determining region 3 (VH-CDR3) comprising an amino acid sequence set forth in SEQ ID NO: 7; a light chain complementarity determining region 1 (VL-CDR1) comprising an amino acid sequence set forth in SEQ ID NO: 9; and a light chain complementarity determining region 2 (VL-CDR2) comprising an amino acid sequence set forth in SEQ ID NO: 11; and a light chain complementarity determining region 3 (VL-CDR3) comprising an amino acid sequence set forth in SEQ ID NO: 13.

In certain embodiments, described herein, is a recombinant antibody that specifically binds Leukemia Inhibitory Factor (LIF) comprising: a heavy chain complementarity determining region 1 (VH-CDR1) comprising an amino acid sequence set forth in SEQ ID NO: 2; a heavy chain complementarity determining region 2 (VH-CDR2) comprising an amino acid sequence set forth in SEQ ID NO: 5; a heavy chain complementarity determining region 3 (VH-CDR3) comprising an amino acid sequence set forth in SEQ ID NO: 6; a light chain complementarity determining region 1 (VL-CDR1) comprising an amino acid sequence set forth in SEQ ID NO: 10; and a light chain complementarity determining region 2 (VL-CDR2) comprising an amino acid sequence set forth in SEQ ID NO: 12; and a light chain complementarity determining region 3 (VL-CDR3) comprising an amino acid sequence set forth in SEQ ID NO: 13. Certain conservative amino acid substitutions are envisioned in the amino acid sequences of the CDRs of this disclosure. In certain embodiments, the antibody comprises CDRs that differ from the amino acid sequence set forth in any one of SEQ ID NOs: 2, 5, 6, 10, 12, and 13 by 1, 2, 3, or 4 amino acids. In certain embodiments, the antibody comprises CDRs that differ from the amino acid sequence set forth in any one of SEQ ID NOs: 2, 5, 6, 10, 12, and 13 by 1, 2, 3, or 4 amino acids and does not affect the binding affinity by greater than 10%, 20%, or 30%.

In certain embodiments, described herein, is a recombinant antibody that specifically binds Leukemia Inhibitory Factor (LIF) comprising: a heavy chain complementarity determining region 1 (VH-CDR1) comprising an amino acid sequence set forth in SEQ ID NO: 3; a heavy chain complementarity determining region 2 (VH-CDR2) comprising an amino acid sequence set forth in SEQ ID NO: 4; a heavy chain complementarity determining region 3 (VH-CDR3) comprising an amino acid sequence set forth in SEQ ID NO: 7; a light chain complementarity determining region 1 (VL-CDR1) comprising an amino acid sequence set forth in SEQ ID NO: 9; and a light chain complementarity determining region 2 (VL-CDR2) comprising an amino acid sequence set forth in SEQ ID NO: 11; and a light chain complementarity determining region 3 (VL-CDR3) comprising an amino acid sequence set forth in SEQ ID NO: 13. Certain conservative amino acid substitutions are envisioned in the amino acid sequences of the CDRs of this disclosure. In certain embodiments, the antibody comprises CDRs that differ from the amino acid sequence set forth in any one of SEQ ID NOs: 3, 4, 7, 9, 11, and 13 by 1, 2, 3, or 4 amino acids. In certain embodiments, the antibody comprises CDRs that differ from the amino acid sequence set forth in any one of SEQ ID NOs: 3, 4, 7, 9, 11, and 13 by 1, 2, 3, or 4 amino acids and does not affect the binding affinity by greater than 10%, 20%, or 30%.

In certain embodiments, described herein, is a therapeutic antibody that specifically binds LIF comprising a humanized heavy chain comprising an amino acid sequence at least about 80%, about 90%, about 95%, about 97%, about 98%, or about 99% identical to the amino acid sequence set forth in any one of SEQ ID NOs: 22-25; and a humanized light chain comprising an amino acid sequence at least about 80%, about 90%, about 95%, about 97%, about 98%, or about 99% identical to the amino acid sequence set forth in any one of SEQ ID NOs: 26-29. In certain embodiments, described herein, is a therapeutic antibody that specifically binds LIF comprising a humanized heavy chain comprising an amino acid sequence set forth in any one of SEQ ID NOs: 22-25; and a humanized light chain comprising an amino acid sequence set forth in any one of SEQ ID NOs: 26-29.

In certain embodiments, described herein, is a therapeutic antibody that specifically binds LIF comprising a humanized heavy chain comprising an amino acid sequence at least about 80%, about 90%, about 95%, about 97%, about 98%, or about 99% identical to the amino acid sequence set forth in SEQ ID NO: 23; and a humanized light chain comprising an amino acid sequence at least about 80%, about 90%, about 95%, about 97%, about 98%, or about 99% identical to the amino acid sequence set forth in of SEQ ID NO: 27. In certain embodiments, described herein, is a therapeutic antibody that specifically binds LIF comprising a humanized heavy chain comprising an amino acid sequence set forth in SEQ ID NO: 23; and a humanized light chain comprising an amino acid sequence set forth in any one of SEQ ID NO: 27.

In certain embodiments, described herein, is a therapeutic antibody that specifically binds LIF comprising a humanized heavy chain comprising an amino acid sequence at least about 80%, about 90%, about 95%, about 97%, about 98%, or about 99% identical to the amino acid sequence set forth in SEQ ID NO: 39; and a humanized light chain comprising an amino acid sequence at least about 80%, about 90%, about 95%, about 97%, about 98%, or about 99% identical to the amino acid sequence set forth in of SEQ ID NO: 27. In certain embodiments, described herein, is a therapeutic antibody that specifically binds LIF comprising a humanized heavy chain comprising an amino acid sequence set forth in SEQ ID NO: 39; and a humanized light chain comprising an amino acid sequence set forth in any one of SEQ ID NO: 27.

### Epitopes Bound by Therapeutically Useful LIF Antibodies

Described herein is a unique epitope of human LIF that when bound inhibits LIF biological activity (e.g., STAT3 phosphorylation) and inhibits tumor growth in vivo and produces a therapeutic effect. The therapeutic antibody of the current disclosure can be a therapeutic antibody that does not comprise the CDRs of h5D8, but binds to the same or similar epitope (amino acid residues) as h5D8. A similar epitope is one that binds within the bounds of the specified epitope. The epitope described herein consists of two discontinuous stretches of amino acids (from residue 13 to residue 32 and from residue 120 to 138 of human LIF), that are present in two distinct topological domains (alpha helixes A and C) of the human LIF protein. This binding is a combination of weak (Van der Waals attraction), medium (hydrogen binding), and strong (salt bridge) interactions. In certain embodiments, a contact residue is a residue on LIF that forms a hydrogen bond with a residue on an anti-LIF antibody. In certain embodiments, a contact residue is a residue on LIF that forms a salt bridge with a residue on an anti-LIF antibody. In certain embodiments, a contact residue is a residue on LIF that results in a Van der Waals attraction with and is within at least 5, 4, or 3 angstroms of a residue on an anti-LIF antibody. The therapeutic antibody can bind this epitope, bind to less of this epitope, or overlap with this epitope and be utilized in the assay described herein.

In certain embodiments, the therapeutic antibody described herein is an isolated antibody that binds any one, two, three, four, five, six, seven, eight, nine, ten, eleven, twelve, thirteen, fourteen, fifteen, sixteen, seventeen, eighteen, nineteen, or twenty of the following residues: A13, I14, R15, H16, P17, C18, H19, N20, Q25, Q29, Q32, D120, R123, S127, N128, L130, C131, C134, S135, or H138 of SEQ ID NO: 40. In certain embodiments, described herein is an isolated antibody that binds all of the following residues: A13, I14, R15, H16, P17, C18, H19, N20, Q25, Q29, Q32, D120, R123, S127, N128, L130, C131, C134, S135, or H138 of SEQ ID NO: 40. In certain embodiments, described herein is an isolated antibody that binds all of the following residues: A13, I14, R15, H16, P17, C18, H19, N20, Q25, Q29, Q32, D120, R123, S127, N128, L130, C131, C134, S135, or H138 of SEQ ID NO: 40. In certain embodiments, the antibody only binds residues that participate with the antibody in strong or medium interactions. In certain embodiments, the antibody only binds residues that participate with the antibody in strong interactions. In a certain embodiment, the antibody interacts with helix A and C of LIF. In a certain embodiment, the antibody blocks LIF interaction with gp130.

### Therapeutic Indications

In certain embodiments, the therapeutic antibodies disclosed herein inhibit LIF signaling in cells. In certain embodiments, the IC₅₀ for biological inhibition of the antibody under serum starved conditions in U-251 cells is less than or equal to about 100, 75, 50, 40, 30, 20, 10, 5, or 1 nanomolar. In certain embodiments, the IC₅₀ for biological inhibition of the antibody under serum starved conditions in U-251 cells is less than or equal to about 900, 800, 700, 600, 500, 400, 300, 200, or 100 nanomolar.

In certain embodiments, disclosed herein, are antibodies useful for the treatment of a cancer or tumor. In certain embodiments, the cancer comprises breast, heart, lung, small intestine, colon, spleen, kidney, bladder, head, neck, ovarian, prostate, brain, pancreatic, skin, bone, bone marrow, blood, thymus, uterine, testicular, and liver tumors. In certain embodiments, tumors which can be treated with the antibodies of the invention comprise adenoma, adenocarcinoma, angiosarcoma, astrocytoma, epithelial carcinoma, germinoma, glioblastoma, glioma, hemangioendothelioma, hemangiosarcoma, hematoma, hepatoblastoma, leukemia, lymphoma, medulloblastoma, melanoma, neuroblastoma, osteosarcoma, retinoblastoma, rhabdomyosarcoma, sarcoma and/or teratoma. In certain embodiments, the tumor/cancer is selected from the group of acral lentiginous melanoma, actinic keratosis, adenocarcinoma, adenoid cystic carcinoma, adenomas, adenosarcoma, adenosquamous carcinoma, astrocytic tumors, Bartholin gland carcinoma, basal cell carcinoma, bronchial gland carcinoma, capillary carcinoid, carcinoma, carcinosarcoma, cholangiocarcinoma, chondrosarcoma, cystadenoma, endodermal sinus tumor, endometrial hyperplasia, endometrial stromal sarcoma, endometrioid adenocarcinoma, ependymal sarcoma, Swing's sarcoma, focal nodular hyperplasia, gastronoma, germ line tumors, glioblastoma, glucagonoma, hemangioblastoma, hemangioendothelioma, hemangioma, hepatic adenoma, hepatic adenomatosis, hepatocellular carcinoma, insulinite, intraepithelial neoplasia, intraepithelial squamous cell neoplasia, invasive squamous cell carcinoma, large cell carcinoma, liposarcoma, lung carcinoma, lymphoblastic leukemia, lymphocytic leukemia, leiomyosarcoma, melanoma, malignant melanoma, malignant mesothelial tumor, nerve sheath tumor, medulloblastoma, medulloepithelioma, mesothelioma, mucoepidermoid carcinoma, myeloid leukemia, neuroblastoma, neuroepithelial adenocarcinoma, nodular melanoma, osteosarcoma, ovarian carcinoma, papillary serous adenocarcinoma, pituitary tumors, plasmacytoma, pseudosarcoma, prostate carcinoma, pulmonary blastoma, renal cell carcinoma, retinoblastoma, rhabdomyosarcoma, sarcoma, serous carcinoma, squamous cell carcinoma, small cell carcinoma, soft tissue carcinoma, somatostatin secreting tumor, squamous carcinoma, squamous cell carcinoma, undifferentiated carcinoma, uveal melanoma, verrucous carcinoma, vagina/vulva carcinoma, VIPpoma, and Wilm's tumor. In certain embodiments, the tumor/cancer to be treated with one or more antibodies of the invention comprise brain cancer, head and neck cancer, colorectal carcinoma, acute myeloid leukemia, pre-B-cell acute lymphoblastic leukemia, bladder cancer, astrocytoma, preferably grade II, III or IV astrocytoma, glioblastoma, glioblastoma multiforme, small cell cancer, and non-small cell cancer, preferably non-small cell lung cancer, lung adenocarcinoma, metastatic melanoma, androgen-independent metastatic prostate cancer, androgen-dependent metastatic prostate cancer, prostate adenocarcinoma, and breast cancer, preferably breast ductal cancer, and/or breast carcinoma. In certain embodiments, the cancer treated with the antibodies of this disclosure comprises glioblastoma. In certain embodiments, the cancer treated with one or more antibodies of this disclosure comprises pancreatic cancer. In certain embodiments, the cancer treated with one or more antibodies of this disclosure comprises ovarian cancer. In certain embodiments, the cancer treated with one or more antibodies of this disclosure comprises lung cancer. In certain embodiments, the cancer treated with one or more antibodies of this disclosure comprises prostate cancer. In certain embodiments, the cancer treated with one or more antibodies of this disclosure comprises colon cancer. In certain embodiments, the cancer treated comprises glioblastoma, pancreatic cancer, ovarian cancer, colon cancer, prostate cancer, or lung cancer. In a certain embodiment, the cancer is refractory to other treatment. In a certain embodiment, the cancer treated is relapsed. In a certain embodiment, the cancer is a relapsed/refractory glioblastoma, pancreatic cancer, ovarian cancer, colon cancer, prostate cancer, genitourinary cancer, gynecologic cancer, gastrointestinal cancer, endocrine system cancer, or lung cancer. In certain embodiments, the cancer comprises an advanced solid tumor, glioblastoma, stomach cancer, skin cancer, prostate cancer, pancreatic cancer, breast cancer, testicular cancer, thyroid cancer, colorectal cancer, bile duct cancer, cervical cancer, endometrial cancer, head and neck cancer, liver cancer, kidney cancer, esophageal cancer, ovarian cancer, colon cancer, lung cancer, lymphoma, or soft tissue cancer. In certain embodiments, the cancer comprises non-small cell lung cancer, epithelial ovarian carcinoma, or pancreatic adenocarcinoma. In certain embodiments, the cancer comprises an advanced solid tumor.

### Therapeutic Methods

In certain embodiments, the therapeutic antibodies can be administered by any route suitable for the administration of antibody-containing pharmaceutical compositions, such as, for example, subcutaneous, intraperitoneal, intravenous, intramuscular, intratumoral, or intracerebral, etc. In certain embodiments, the antibodies are administered intravenously. In certain embodiments, the antibodies are administered on a suitable dosage schedule, for example, weekly, twice weekly, monthly, twice monthly, etc. In certain embodiments, the antibodies are administered once every three weeks. The antibodies can be administered in any therapeutically effective amount. In certain embodiments, the therapeutically acceptable amount is between about 0.1 mg/kg and about 50 mg/kg. In certain embodiments, the therapeutically acceptable amount is between about 1 mg/kg and about 40 mg/kg. In certain embodiments, the therapeutically acceptable amount is between about 5 mg/kg and about 30 mg/kg. The therapeutic antibody can be administered at a flat dose regardless of the weight or mass of the individual to whom the h5D8 antibody is administered. The h5D8 antibody can be administered at a flat dose regardless of the weight or mass of the individual to whom the therapeutic antibody is administered, provided that the individual has a mass of at least about 37.5 kilograms. A flat dose of therapeutic antibody can be administered from about 75 milligrams to about 2000 milligrams. A flat dose of therapeutic antibody can be administered from about 225 milligrams to about 2000 milligrams, from about 750 milligrams to about 2000 milligrams, from about 1125 milligrams to about 2000 milligrams, or from about 1500 milligrams to about 2000 milligrams. A flat dose of therapeutic antibody can be administered at about 75 milligrams. A flat dose of therapeutic antibody can be administered at about 225 milligrams. A flat dose of therapeutic antibody can be administered at about 750 milligrams. A flat dose of therapeutic antibody can be administered at about 1125 milligrams. A flat dose of therapeutic antibody can be administered at about 1500 milligrams. A flat dose of therapeutic antibody can be administered at about 2000 milligrams.

Other dosages of therapeutic antibody are contemplated. A flat dose of therapeutic antibody can be administered at about 50, 100, 150, 175, 200, 250, 300, 325, 350, 375, 400, 425, 450, 475, 500, 525, 550, 575, 600, 625, 650, 675, 700, 725, 775, 800, 825, 850, 875, 900, 925, 950, 975, 1000, 1025, 1050, 1075, 1100, 1150, 1175, 1200, 1225, 1250, 1275, 1300, 1325, 1350, 1375, 1400, 1425, 1450, 1475, 1525, 1550, 1575, 1600, 1625, 1650, 1675, 1700, 1725, 1750, 1775, 1800, 1825, 1850, 1875, 1900, 1925, 1950, 1975, 2025, 2050, 2075, or 2100 milligrams. Any of these doses can be administered once a week, once every two weeks, once every three weeks, or once every four weeks.

The therapeutic antibody can be administered at a dose based on the bodyweight or mass of the individual to whom the therapeutic antibody is administered. A body weight adjusted dose of therapeutic antibody can be administered from about 1 mg/kg to about 25 mg/kg. A body weight adjusted dose of therapeutic antibody can be administered from about 3 mg/kg to about 25 mg/kg, from about 10 mg/kg to about 25 mg/kg, from about 15 mg/kg to about 25 mg/kg, or from about 20 mg/kg to about 25 mg/kg. A body weight adjusted dose of h5D8 can be administered at about 1 mg/kg. A body weight adjusted dose of therapeutic antibody can be administered at about 3 mg/kg. A body weight adjusted dose of therapeutic antibody can be administered at about 10 mg/kg. A body weight adjusted dose of therapeutic antibody can be administered at about 15 mg/kg. A body weight adjusted dose of therapeutic antibody can be administered at about 20 mg/kg. A body weight adjusted dose of therapeutic antibody can be administered at about 25 mg/kg.

Any of the doses detailed herein can be administered i.v. over a time period of at least about 60 minutes; however, this period can vary somewhat based upon conditions relevant to each individual administration.

### Pharmaceutically Acceptable Excipients, Carriers and Diluents

In certain embodiments, the antibodies of the current disclosure are administered suspended in a sterile solution. In certain embodiments, the solution comprises a physiologically appropriate salt concentration (e.g., NaCl). In certain embodiments, the solution comprises between about 0.6% and 1.2% NaCl. In certain embodiments, the solution comprises between about 0.7% and 1.1% NaCl. In certain embodiments, the solution comprises between about 0.8% and 1.0% NaCl. In certain embodiments, a highly concentrated stock solution of antibody may be diluted in about 0.9% NaCl. In certain embodiments, the solution comprises about 0.9% NaCl. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), polysorbate and poloxamer 188; polyol/disaccharide/polysaccharides, for example, glucose, dextrose, mannose, mannitol, sorbitol, sucrose, trehalose, and dextran 40; amino acids, for example, histidine, glycine or arginine; antioxidants, for example, ascorbic acid, methionine; and chelating agents, for example, EGTA or EGTA. In certain embodiments, the antibodies of the current disclosure are shipped/stored lyophilized and reconstituted before administration. In certain embodiments, lyophilized antibody formulations comprise a bulking agent such as, mannitol, sorbitol, sucrose, trehalose, and dextran 40. In a certain embodiment, anti-LIF antibodies of this disclosure can be shipped and stored as a concentrated stock solution to be diluted at the treatment site of use. In certain embodiments, the stock solution comprises about 25 mM histidine, about 6% sucrose, about 0.01% polysorbate, and about 20 mg/mL of anti-LIF antibody. In certain embodiments, the pH of the solution is about 6.0. In certain embodiments, the form administered to an individual is an aqueous solution comprising about 25 mM histidine, about 6% sucrose, about 0.01% polysorbate 80, and about 20 mg/mL of h5D8 antibody. In certain embodiments, the pH of the solution is about 6.0.

### EXAMPLES

The following illustrative examples are representative of embodiments of the compositions and methods described herein and are not meant to be limiting in any way.

### Example 1-Generation of Rat Antibodies Specific for LIF

A cDNA encoding amino acids 23-202 of human LIF was cloned into expression plasmids (Aldevron GmbH, Freiburg, Germany). Groups of laboratory rats (Wistar) were immunized by intradermal application of DNA-coated gold-particles using a hand-held device for particle-bombardment (“gene gun”). Cell surface expression on transiently transfected HEK cells was confirmed with anti-tag antibodies recognizing a tag added to the N-terminus of the LIF protein. Serum samples were collected after a series of immunizations and tested in flow cytometry on HEK cells transiently transfected with the aforementioned expression plasmids. Antibody-producing cells were isolated and fused with mouse myeloma cells (Ag8) according to standard procedures. Hybridomas producing antibodies specific for LIF were identified by screening in a flow cytometry assay as described above. Cell pellets of positive hybridoma cells were prepared using an RNA protection agent (RNAlater, cat. #AM7020 by ThermoFisher Scientific) and further processed for sequencing of the variable domains of the antibodies.

### Example 2-Generation of Mouse Antibodies Specific for LIF

A cDNA encoding amino acids 23-202 of human LIF was cloned into expression plasmids (Aldevron GmbH, Freiburg, Germany). Groups of laboratory mice (NMRI) were immunized by intradermal application of DNA-coated gold-particles using a hand-held device for particle-bombardment (“gene gun”). Cell surface expression on transiently transfected HEK cells was confirmed with anti-tag antibodies recognizing a tag added to the N-terminus of the LIF protein. Serum samples were collected after a series of immunizations and tested in flow cytometry on HEK cells transiently transfected with the aforementioned expression plasmids. Antibody-producing cells were isolated and fused with mouse myeloma cells (Ag8) according to standard procedures. Hybridomas producing antibodies specific for LIF were identified by screening in a flow cytometry assay as described above. Cell pellets of positive hybridoma cells were prepared using an RNA protection agent (RNAlater, cat. #AM7020 by ThermoFisher Scientific) and further processed for sequencing of the variable domains of the antibodies.

### Example 3-Humanization of Rat Antibodies Specific for LIF

One clone from the rat immunization (5D8) was chosen for subsequent humanization. Humanization was conducted using standard CDR grafting methods. The heavy chain and light chain regions were cloned from the 5D8 hybridoma using standard molecular cloning techniques and sequenced by the Sanger method. A BLAST search was then conducted against human heavy chain and light chain variable sequences and 4 sequences from each were chosen as acceptor frameworks for humanization. These acceptor frameworks were deimmunized to remove T cell response epitopes. The heavy chain and light chain CDR1, CDR2 and CDR3 of 5D8 were cloned into the 4 different heavy chain acceptor frameworks (H1 to H4), and 4 different light chain frameworks (L1 to L4). Then all 16 different antibodies were tested for: expression in CHO-S cells (Selexis); inhibition of LIF-induced STAT3 phosphorylation; and binding affinity by Surface Plasmon Resonance (SPR). These experiments are summarized in Table 1.

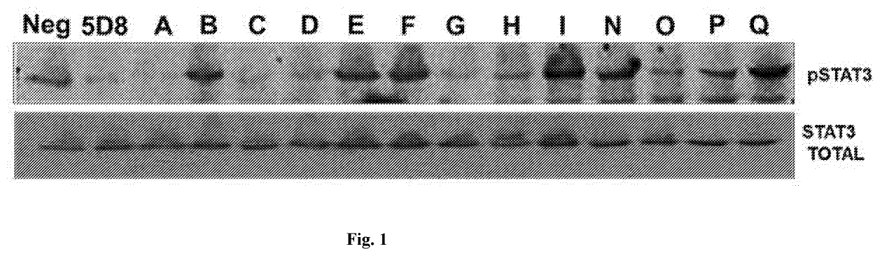

TABLE 1
Summary of 5D8 humanization

| Heavy chain | Inhibition of LIF- | | |
|---|---|---|---|
| light chain | induced pSTAT3 | Affinity by | Expression |
| combination | from FIG. 1 | SPR K_{D1} (pM) | (ug/mL) |
| H0L0 | +++ | 133 ± 46 | 393 |
| H1L1 | − | N/A | 627 |
| H1L2 | +++ | 55 ± 23 | 260 |
| H1L3 | +++ | 54 ± 31 | 70 |
| H1L4 | − | N/A | 560 |
| H2L1 | − | N/A | 369 |
| H2L2 | +++ | 52 ± 22 | 392 |
| H2L3 | ++ | 136 ± 19 | 185 |
| H2L4 | − | N/A | 78 |
| H3L1 | N/A | N/A | No expression |
| H3L2 | N/A | N/A | No expression |
| H3L3 | N/A | N/A | No expression |
| H3L4 | N/A | N/A | No expression |
| H4L1 | − | N/A | 259 |
| H4L2 | ++ | 913 ± 308 | 308 |
| H4L3 | + | | 252 |
| H4L4 | − | N/A | 186 |

N/A = Not attempted;
H0L0 = chimeric antibody with full rat heavy and light chain variable regions

The expression performance of the transfected cells was compared in Erlenmeyer flasks (seeding 3×10⁵ cells/mL, 200 mL culture volume) within fed-batch cultivation after 10 days of cell culture. At this point cells were harvested and the secreted antibody purified using a Protein A column and then quantitated. All humanized antibodies expressed except those using the H3 heavy chain. The H2 and L2 variable regions performed well compared to other variable regions (SEQ ID NO: 15 and SEQ ID NO: 19).

Inhibition of LIF-induced STAT3 phosphorylation at tyrosine 705 was determined by western blot. U251 glioma cells were plated in 6-well plates at a density of 100,000 cells/well. Cells were cultured in complete medium for 24 hours before any treatment and after that, cells were serum starved for 8 hours. After that, cells with the indicated antibodies over night at a concentration of 10 μg/ml. After treatment, proteins were obtained in radio-immunoprecipitation assay (RIPA) lysis buffer containing phosphatase and protease inhibitors, quantified (BCA-protein assay, Thermo Fisher Scientific) and used in western blot. For western blot, membranes were blocked for 1 hour in 5% non-fat dried milk—TBST and incubated with the primary antibody overnight (p-STAT3, catalog #9145, Cell Signaling or STAT3, catalog #9132, Cell Signaling) or 30 minutes (β-actin-peroxidase, catalog #A3854, Sigma-Aldrich). Membranes were then washed with TBST, incubated with secondary and washed again. Proteins were detected by chemiluminescence (SuperSignal Substrate, catalog #34076, Thermo Fisher Scientific). These results are shown in FIG. 1. The darker the pSTAT3 band the less inhibition is present. Inhibition was high in lanes labeled 5D8 (non humanized rat), A (H0L0), C (H1L2), D (H1L3), and G (H2L2); inhibition was moderate in H (H2L3), O (H4L2), and P (H4L3); inhibition was absent in B (H1L1), E (H1L4), F (H2L1), I (H2L4), N (H4L1) and Q (H4L4).

Antibodies that exhibited inhibition of LIF-induced STAT3 phosphorylation were then analyzed by SPR to determine binding affinity. Briefly, binding of the A (H0L0), C (H1L2), D (H1L3), and G (H2L2), H (H2L3) and O (H4L2) humanized antibodies to amine coupled hLIF was observed using a Biacore™ 2002 Instrument. Kinetic constants and affinities were determined by mathematical sensorgram fitting (Langmuir interaction model [A+B=AB]) of all sensorgrams generated on all sensor chip surfaces at six ligand concentrations. The best fitted curves (minimal Chi2) of each concentration were used for calculation of kinetic constants and affinities. See Table 1.

Since the experimental setup used bivalent antibodies as analytes, best fitted sensorgrams, were also analyzed on basis of a bivalent analyte fitting model [A+B=AB; AB+B=AB2] in order to obtain a more detailed insight into the target binding mechanism of the humanized antibodies. Kinetic sensorgram analysis using a bivalent fitting model [A+B=AB; AB+B=AB2] confirmed the relative affinity ranking of the mAb samples.

The humanized 5D8 comprising H2 and L2 was selected for more in-depth analysis due to its high binding affinity and high yield from batch culture.

### Example 4-Humanization of Clone 5D8 Improves Binding to LIF

The H2L2 clone (h5D8) was selected for further analysis and compared binding by SPR to the parental rat 5D8 (r5D8) and a mouse clone 1B2. The 1B2 antibody is a previously disclosed mouse anti-LIF antibody previously deposited at the Deutsche Sammlung von Mikroorganismen and Zellkulturen GmbH (DSM ACC3054) and was included for comparison purposes. Recombinant human LI, purified from E. coli and HEK-293 cells, respectively, were used as ligands. The LIF from human or E. coli sources was covalently coupled to the surface of Biacore optical sensor chips using amine coupling chemistry, and binding affinities were calculated from the kinetic constants.

### Materials and Methods

Human LIF from E. coli was obtained from Millipore, reference LIF 1010; human LIF from HEK-293 cells was obtained from ACRO Biosystems, reference LIF-H521b. LIF was coupled to the sensor chips using the Biacore Amine Coupling Kit (BR-1000-50; GE-Healthcare, Uppsala). Samples were run on a Biacore™ 2002 Instrument using CM5 optical sensor chips (BR-1000-12; GE-Healthcare, Uppsala). Biacore HBS-EP buffer was used during the machine runs (BR-1001-88; GE-Healthcare, Uppsala). Kinetic analysis of binding sensorgrams was performed using BIAevaluation 4.1 software. Kinetic constants and affinities were determined by mathematical sensorgram fitting (Langmuir interaction model [A+B=AB]) of all sensorgrams generated on all sensor chip surfaces at increasing analyte concentrations. Sensorgrams were also analyzed on the basis of a bivalent analyte sensorgram fitting model [A+B=AB; AB+B=AB₂], including component analysis, in order to generate an estimate on the bivalent contribution to the determined Langmuir antibody—target affinities (e.g., avidity contribution). The best fitted curves (minimal Chi²) of each concentration were used for calculation of kinetic constants and affinities. Summaries of these affinity experiments are shown in Table 2 (human LIF made in E. coli) and Table 3 (human LIF made in HEK 293 cells).

TABLE 2

| Improved binding | | |
|---|---|---|
| of 5D8 after | K_{D} [pM] | |
| humanization | Langmuir 1:1 | Bivalent analyte |
| hLIF (E. coli) | sensorgram fitting | fitting |
| Mouse 1B2 | 400 ± 210 | 1500 ± 200 |
| r5D8 (Rat) | 130 ± 30 | 780 ± 130 |
| h5D8 (humanized) | 26 ± 14 | 82 ± 25 |

TABLE 3

| Improved binding | | |
|---|---|---|
| of 5D8 after | K_{D} [pM] | |
| humanization | Langmuir 1:1 | Bivalent analyte |
| hLIF (HEK 293) | sensorgram fitting | fitting |
| Mouse 1B2 | 320 ± 150 | 3900 ± 900 |
| r5D8 (rat) | 135 ± 100 | 410 ± 360 |
| h5D8 (humanized) | 13 ± 6 | 63 ± 30 |

The Langmuir 1:1 sensorgram fitting model from this set of experiments indicates that the humanized 5D8 (h5D8) antibody bound with ˜10-25 times higher affinity to human LIF than mouse 1B2 and r5D8.

Next, the h5D8 antibody was tested against LIF of multiple species by SPR. h5D8 SPR binding kinetics were performed for recombinant LIF analytes derived from different species and expression systems: human LIF (E. coli, HEK293 cells); mouse LIF (E. coli, CHO cells); rat LIF (E. coli); cynomolgus monkey LIF (yeast, HEK293 cells).

### Materials and Methods

The h5D8 antibody was immobilized to the sensor chip surface by non covalent, Fc specific capturing. Recombinant, Ig(Fc) specific S. aureus Protein A/G was used as capturing agent, allowing sterically uniform and flexible presentation of the anti-LIF antibody to the LIF analytes. Sources of the LIF analytes are as follows: Human LIF (from E. coli; Millipore reference LIF 1050); Human LIF (from HEK cells ACRO Biosystems LIF-H521); Mouse LIF (E. coli; Millipore Cat. No NF-LIF2010); Mouse LIF (from CHO cells; Reprokine Catalog #RCP09056); Monkey LIF (yeast Kingfisher Biotech Catalog #RP1074Y); Monkey LIF produced in HEK-293 cell. Overall h5D8 exhibited binding to LIF from several species. A summary of this affinity experiment is shown in Table 4.

TABLE 4

| Broad species reactivity | Langmuir 1:1 sensorgram fitting | | |
|---|---|---|---|
| of humanized 5D8 | mean Kₐ | mean K_{d} (1/S) | mean K_{D} |
| Analyte | (1/Ms)[10⁵] | [10⁻⁵] | [pM] |
| Human LIF (E.coli) | 8.5 ± 0.7 | 7.2 ± 0.7 | 86 ± 9 |
| Human LIF (HEK-293) | 5.5 ± 0.02 | 3.1 ± 0.7 | 56 ± 13 |
| Mouse LIF (E.coli) | 21.4 ± 3.7 | 5.7 ± 1.0 | 27 ± 6 |
| Mouse LIF(CHO cells) | 6.5 ± 0.7 | 1.1 ± 0.3 | 17 ± 4 |
| Cyno Monkey LIF (yeast) | 6.3 ± 0.8 | 5.4 ± 0.7 | 89 ± 10 |
| Cyno Monkey LIF (HEK-293) | 2.4 ± 0.2 | 3.3 ± 0.3 | 134 ± 6 |

### Example 5-Humanized Clone 5D8 Inhibits LIF-Induced Phosphorylation of STAT3 In Vitro

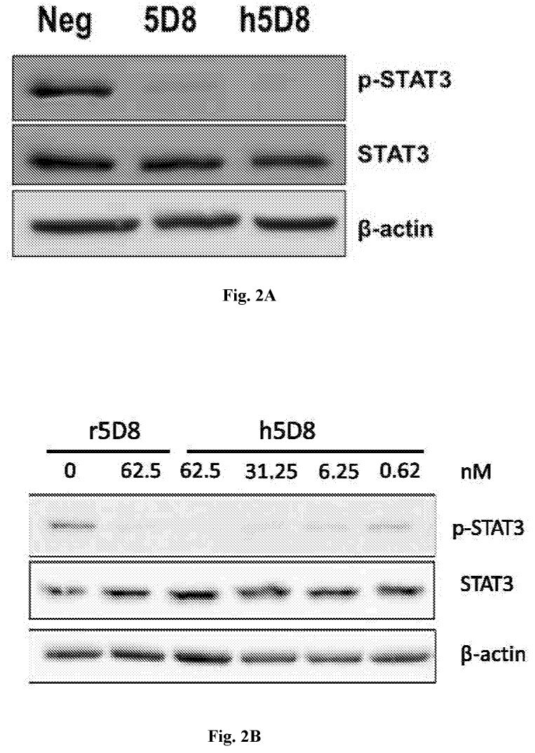

To determine the biological activity of h5D8, the humanized and parental versions were tested in a cell culture model of LIF activation. FIG. 2A shows that the humanized clone exhibited increased inhibition of STAT3 phosphorylation (Tyr 705) when a glioma cell line was incubated with human LIF. FIG. 2B shows an experiment with the same set up of FIG. 2A repeated with different dilutions of the h5D8 antibody.

### Methods

U251 glioma cells were plated in 6-well plates at a density of 150,000 cells/well. Cells were cultured in complete medium for 24 hours before any treatment. After that, cells were treated over night or not (control cells) with r5D8 anti-LIF antibody or h5D8 anti-LIF antibody at a concentration of 10 μg/ml.

After treatment, proteins were obtained in radio-immunoprecipitation assay (RIPA) lysis buffer containing phosphatase and protease inhibitors, quantified (BCA-protein assay, Thermo Fisher Scientific) and used in western blot. For western blot, membranes were blocked for 1 hour in 5% non-fatty milk—TBST and incubated with the primary antibody overnight (p-STAT3, catalog #9145, Cell Signaling or STAT3, catalog #9132, Cell Signaling) or 30 minutes (β-actin-peroxidase, catalog #A3854, Sigma-Aldrich). Membranes were then washed with TBST, incubated with secondary antibody if necessary, and washed again. Proteins were detected by chemiluminescence (SuperSignal Substrate, catalog #34076, Thermo Fisher Scientific).

### Example 6-IC₅₀ Value of h5D8 Antibody Treatment on Endogenous Levels of LIF in U-251 Cells

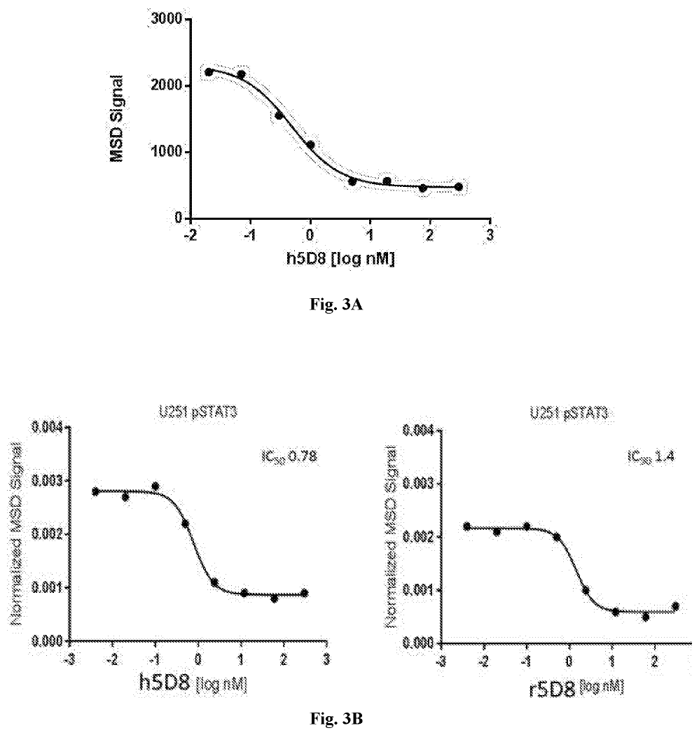

An IC₅₀ of as low as 490 picomolar (FIG. 3A) was determined for biological inhibition for h5D8 under serum starved conditions in U-251 cells. See representative results FIGS. 3A and 3B and Table 5.

TABLE 5

| | | | | | |
|---|---|---|---|---|---|
| Origin of | Cell Line | | | | JAK inhibition |
| Cell Line | Name | Treatment | IC₅₀ (nM) | IC₉₀ (nM) | (%) |

| Endogenous LIF Condition | | | n = 1 | n = 2 | Mean | SD | Mean | Mean |
|---|---|---|---|---|---|---|---|---|
| GBM | U251 | h5D8 | 0.78 | 0.54 | 0.66 | 0.12 | 4.1 | 84% |
| | | r5D8 | 1.6 | 1.5 | 1.4 | 0.15 | 8.5 | 86% |
| | | | 1.2 | 1.4 | | | | |

### Methods

The U-251 cells were seeded at 600,000 cells per 6 cm plate (per condition). Cells were treated with h5D8 in corresponding concentration (titration) overnight at 37° C., under serum starvation (0.1% FBS). As a positive control for pSTAT3, recombinant LIF (R&D #7734-LF/CF) was used to stimulate the cells at 1.79 nM for 10 min at 37° C. As a negative control of pSTAT3, the JAK I inhibitor (Calbiochem #420099) was used at 1 uM for 30 min at 37° C. Cells were then harvested on ice for lysates following the Meso Scale Discovery Multi-Spot Assay System Total STAT3 (Cat #K150SND-2) and Phospho-STAT3 (Tyr705) (Cat #K150SVD-2) kits' protocol, to measure protein levels detectable by the MSD Meso Sector S600.

### Example 7-Additional Antibodies that Specifically Bind to Human LIF

Other rat antibody clones (10G7 and 6B5) that specifically bind human LIF were identified and a summary of their binding characteristics are shown below in Table 6, clone 1B2 served as a comparison.

### Methods

Kinetic real time binding analysis was performed for anti-LIF mAbs 1B2, 10G7 and 6B5, immobilized on the surface of CM5 optical sensor chips, applying recombinant LIF target proteins [human LIF (E. coli); Millipore Cat. No. LIF 1010 and human LIF (HEK293 cells); ACRO Biosystems Cat. No. LIF-H521b] as analytes.

Kinetic constants and affinities were obtained by mathematical sensorgram fitting using a Langmuir 1:1 binding model applying global (simultaneous fitting of sensorgram sets) as well as single curve fitting algorithms. Plausibility of global fits was assessed by k_{obs} analysis.

TABLE 6

| Affinity | | | | |
|---|---|---|---|---|
| measurements of | | | | |
| additional anti-LIF | | | | |
| antibodies | | Langmuir 1:1 sensorgram fitting | | |
| Analyte | clone | mean Kₐ (1/Ms) | mean K_{d} (1/S) | mean K_{D} [nM] |
| Human LIF | 1B2 | 1.1 ± 0.4E5 | 1.1 ± 0.3E−3 | 9.7 ± 1.4 |
| (E. coli) | | | | |
| Human LIF | 1B2 | 2.0 ± 0.04E6 | 1.4 ± 0.2E−3 | 0.7 ± 0.03 |
| (HEK-293) | | | | |
| Human LIF | 10G7 | 7.9 ± 5.8E4 | 6.0 ± 2.3E−4 | 12.6 ± 9.5 |
| (E. coli) | | | | |
| Human LIF | 10G7 | 3.6 ± 1.75E5 | 3.1 ± 0.5E−4 | 1.1 ± 0.6 |
| (HEK-293) | | | | |
| Human LIF | 6B5 | N/A | N/A | N/A |
| (E. coli) | | | | |
| Human LIF | 6B5 | 3.6 ± 1.7E5 | 3.1 ± 0.5E4 | 62 ± 6 |
| (HEK-293) | | | | |

### Example 8-Additional Anti LIF Antibodies Inhibit LIF-Induced Phosphorylation of STAT3 In Vitro

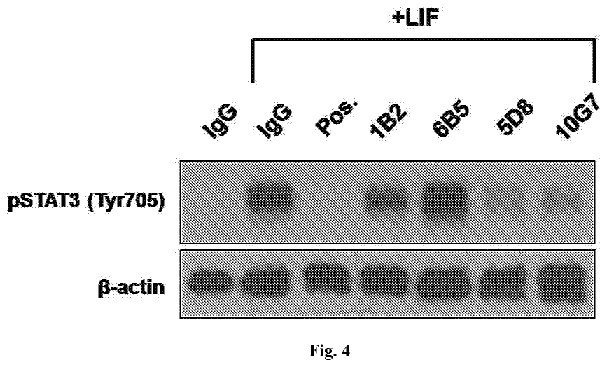

Additional clones were tested for their ability to inhibit LIF-induced phosphorylation of STAT3 in cell culture. As shown in FIG. 4 clones 10G7 and the previously detailed r5D8 exhibited high inhibition of LIF-induced STAT3 phosphorylation, compared to the 1B2 clone. Anti-LIF polyclonal anti-sera (pos.) was included as a positive control While 6B5 exhibited no inhibition, this may be explained by a possible lack of 6B5 binding to non-glycosylated LIF which was used in this experiment.

### Methods

Patient derived glioma cells were plated in 6-well plates at a density of 150,000 cells/well. Cells were cultured in GBM medium that consisted of Neurobasal medium (Life Technologies) supplemented with B27 (Life Technologies), penicillin/streptomycin and growth factors (20 ng/ml EGF and 20 ng/ml FGF-2 [PeproTech]) for 24 hours before any treatment. The following day, cells were treated or not with recombinant LIF produced in E. coli or a mix of recombinant LIF plus the indicated antibodies for 15 minutes (final concentration of 10 μg/ml for the antibodies and 20 ng/ml of recombinant LIF). After treatment, proteins were obtained in radio-immunoprecipitation assay (RIPA) lysis buffer containing phosphatase and protease inhibitors, quantified (BCA-protein assay, Thermo Fisher Scientific) and used in western blot. For western blot, membranes were blocked for 1 hour in 5% non-fatty milk—TBST and incubated with the primary antibody overnight (p-STAT3, catalog #9145, Cell Signaling) or 30 minutes (β-actin-peroxidase, catalog #A3854, Sigma-Aldrich). Membranes were then washed with TBST, incubated with secondary antibody if necessary, and washed again. Proteins were detected by chemiluminescence (SuperSignal Substrate, catalog #34076, Thermo Fisher Scientific).

### Example 9—LIF is Highly Overexpressed Across Multiple Tumor Types

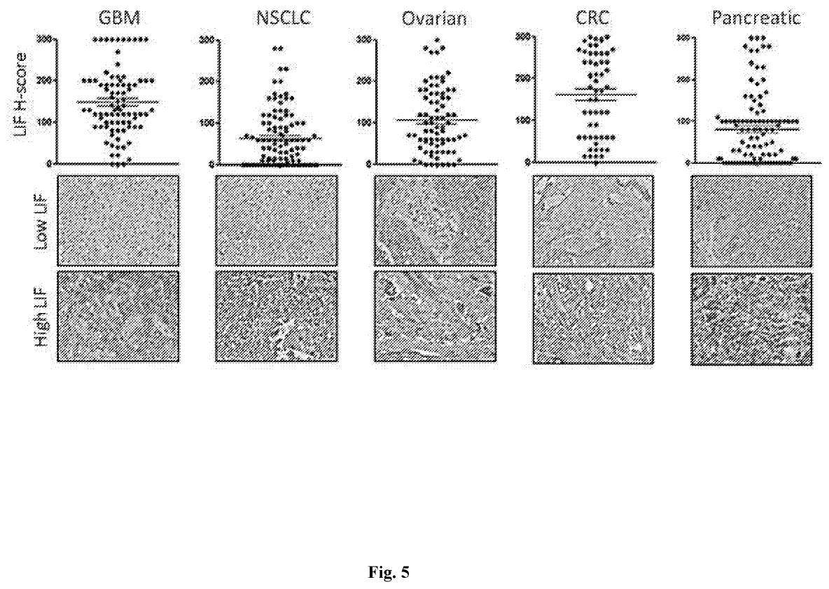

Immunohistochemistry was conducted on multiple human tumor types to determine the degree of LIF expression. As shown in FIG. 5 LIF is highly expressed in glioblastoma multiforme (GBM), non-small cell lung cancer (NSCLC), ovarian cancer, colorectal cancer (CRC), and pancreatic tumors.

### Example 10-Humanized Clone h5D8 Inhibits Tumor Growth in a Mouse Model of Non-Small Cell Lung Carcinoma

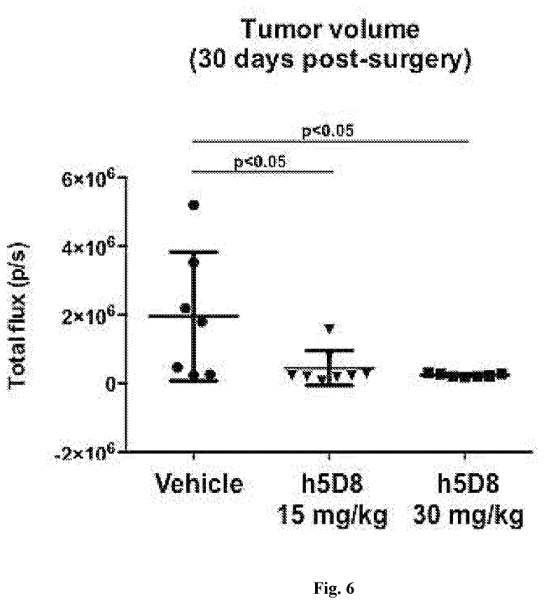

To determine the ability of the humanized 5D8 clone to inhibit a LIF positive cancer in vivo this antibody was tested in a mouse model of non-small cell lung carcinoma (NSCLC). FIG. 6 shows reduced tumor growth in mice treated with this antibody compared to a vehicle negative control.

### Methods

The murine non-small cell lung cancer (NSCLC) cell line KLN205 with high LIF levels was stably infected with lentivirus expressing the firefly luciferase gene for in vivo bioluminescence monitoring. To develop the mouse model, 5×10⁵ KLN205 non-small cell lung cancer (NSCLC) cells were orthotopically implanted into the left lung of 8-week-old immunocompetent syngeneic DBA/2 mice by intercostal puncture. Mice were treated with a control vehicle or with 15 mg/kg or 30 mg/kg of the h5D8 antibody intraperitoneally twice a week and tumor growth was monitored by bioluminescence. For the bioluminescence imaging, mice received an intraperitoneal injection of 0.2 mL of 15 mg/mL D-luciferin under 1-2% inhaled isoflurane anesthesia. The bioluminescence signals were monitored using the IVIS system 2000 series (Xenogen Corp., Alameda, Calif., USA) consisting of a highly sensitive cooled CCD camera. Living Image software (Xenogen Corp.) was used to grid the imaging data and integrate the total bioluminescence signals in each boxed region. Data were analyzed using the total photon flux emission (photons/second) in the regions of interest (ROI). The results demonstrate that treatment with the h5D8 antibody promote tumor regression. Data are presented as mean±SEM.

### Example 11—h5D8 Inhibits Tumor Growth in a Mouse Model of Glioblastoma Multiforme

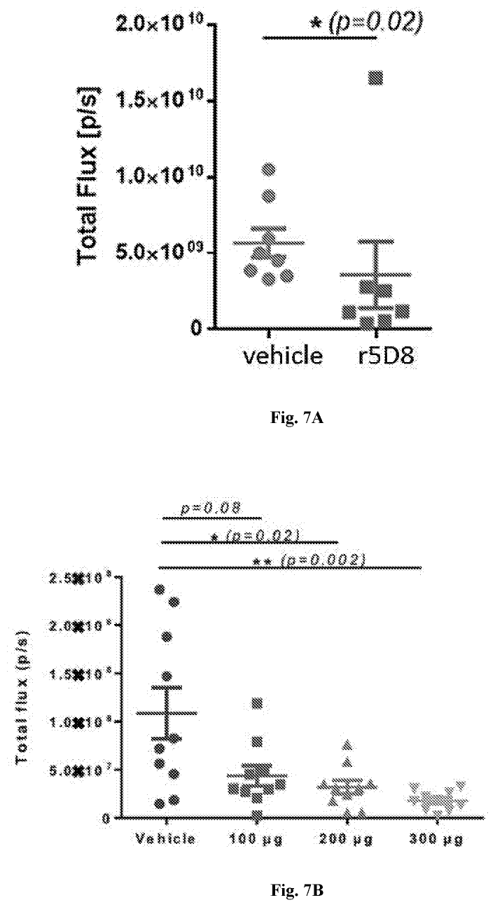

In an orthotopic GBM tumor model using a luciferase expressing human cell line U251, r5D8 significantly reduced tumor volumes in mice administered 300 μg r5D8 and h5D8 by intraperitoneal (IP) injection twice a week. Results of this study are shown in FIG. 7A (quantitation at day 26 post treatment). This experiment was also conducted using humanized h5D8 mice treated with 200 μg or 300 μg showed a statistically significant reduction in tumor after 7 days of treatment.

### Methods

U251 cells stably expressing luciferase were harvested, washed in PBS, centrifuged at 400 g for 5 min, resuspended in PBS and counted with an automated cell counter (Countess, Invitrogen). Cells were kept on ice to maintain optimal viability. Mice were anaesthetized with intraperitoneal administration of Ketamine (Ketolar50®)/Xylacine (Rompún®) (75 mg/kg and 10 mg/kg respectively). Each mouse was carefully placed in the stereotactic device and immobilized. Hair from the head was removed with depilatory cream, and the head skin was cut with a scalpel to expose the skull. A small incision was carefully made with a drill in the coordinates 1.8 mm lateral and 1 mm anterior to the Lambda. 5 μL of cells were inoculated using a Hamilton 30G syringe into the right corpus striatum, at 2.5 mm of depth. Head incision was closed with Hystoacryl tissue adhesive (Braun) and mice were injected with subcutaneous analgesic Meloxicam (Metacam®) (1 mg/kg). The final cell number implanted into each mouse was 3×10⁵.

Mice were treated twice a week with h5D8 administered intraperitoneally. Treatment was initiated on day 0, immediately after tumor cell inoculation. Mice received a total of 2 doses of h5D8 or vehicle control.

Body weight and tumor volume: Body weight was measured 2 times/week and tumor growth was quantified by bioluminescence on day 7 (Xenogen IVIS Spectrum). To quantify bioluminescence activity in vivo, mice were anaesthetized using isofluorane, and injected intraperitoneally with luciferin substrate (PerkinElmer) (167 μg/kg).

Tumor size as determined by bioluminescence (Xenogen IVIS Spectrum) was evaluated at day 7. The individual tumor measurements and mean±SEM for each treatment group were calculated. Statistical significance was determined by the unpaired non-parametric Mann-Whitney U-test.

### Example 12—h5D8 Inhibits Tumor Growth in a Mouse Model of Ovarian Cancer

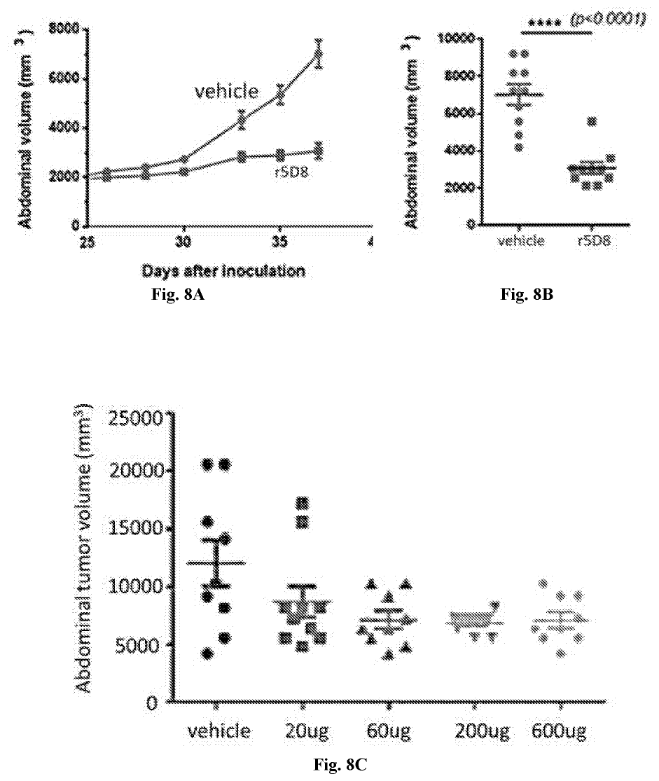

The efficacy of r5D8 was evaluated in two other syngeneic tumor models. In the ovarian orthotopic tumor model ID8, IP administration of 300 μg r5D8 twice weekly significantly inhibited tumor growth as measured by abdominal volume (FIGS. 8A and 8B). Results in FIG. 8C show that h5D8 also reduced tumor volume at a dose of 200 μg and above.

### Methods

ID8 cells were cultured in Dulbecco's Modified Eagle Medium (DMEM) (Gibco, Invitrogen), supplemented with 10% Fetal Bovine Serum (FBS) (Gibco, Invitrogen), 40 U/mL Penicillin and 40 μg/mL Streptomycin (PenStrep) (Gibco, Invitrogen) and 0.25 μg/mL Plasmocin (Invivogen).

The ID8 cells were harvested, washed in PBS, centrifuged at 400 g for 5 min and resuspended in PBS. Cells were kept on ice to maintain optimal viability and 200 μL of the cell suspension was injected intraperitoneally with a 27G needle. The final cell number implanted into mice was 5×10⁶.

Mice were treated twice weekly with h5D8 administered ip at different doses as indicated. Body weights were measured 2 times/week and tumor progression was monitored by measuring abdominal girth using a caliper (Fisher Scientific).

### Example 13—r5D8 Inhibits Tumor Growth in a Mouse Model of Colorectal Cancer

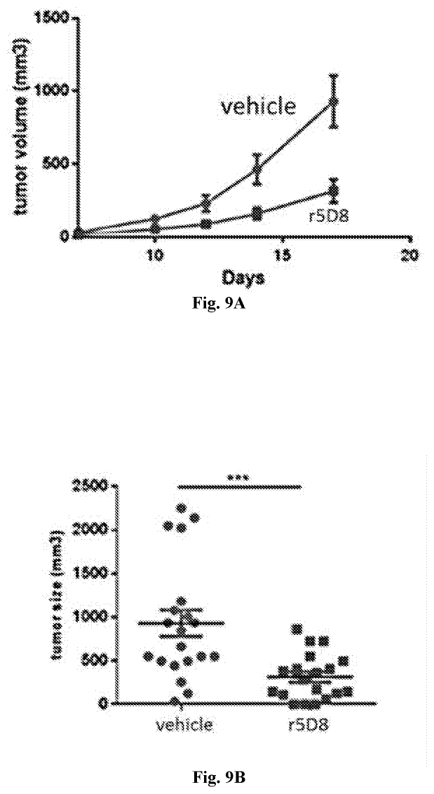

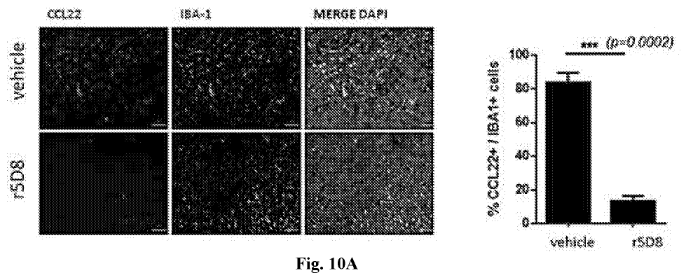

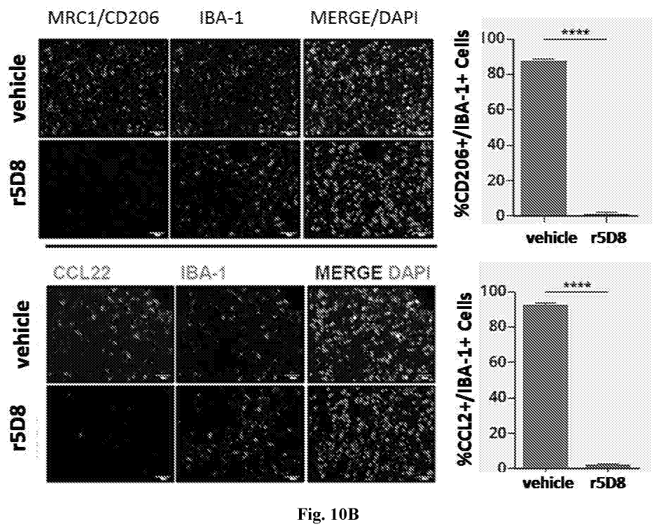

In mice with subcutaneous colon CT26 tumors, r5D8 (administered 300 μg IP twice weekly) significantly inhibited tumor growth (FIGS. 9A and 9B).

### Methods

CT26 cells were cultured in Roswell Park Memorial Institute medium (RPMI [Gibco, Invitrogen]), supplemented with 10% Fetal Bovine Serum (FBS), 40 U/mL penicillin and 40 μg/mL streptomycin (PenStrep) and 0.25 μg/mL Plasmocin.

CT26 cells (8×10⁵) were trypsinized, rinsed with PBS, centrifuged at 400 g for 5 minutes and resuspended in 100 μL PBS. Cells were kept on ice to avoid cell death. The CT26 cells were administered to mice via subcutaneous injection using a 27G needle.

300 μg r5D8, or vehicle control, was administered to the mice via intraperitoneal injection (IP) twice weekly from day 3 post CT26 cell implant.

Body weight and tumor volumes were measured three times per week. Tumor volume was measured using a caliper (Fisher Scientific).

### Example 14—r5D8 Reduces Inflammatory Infiltration in Tumor Models

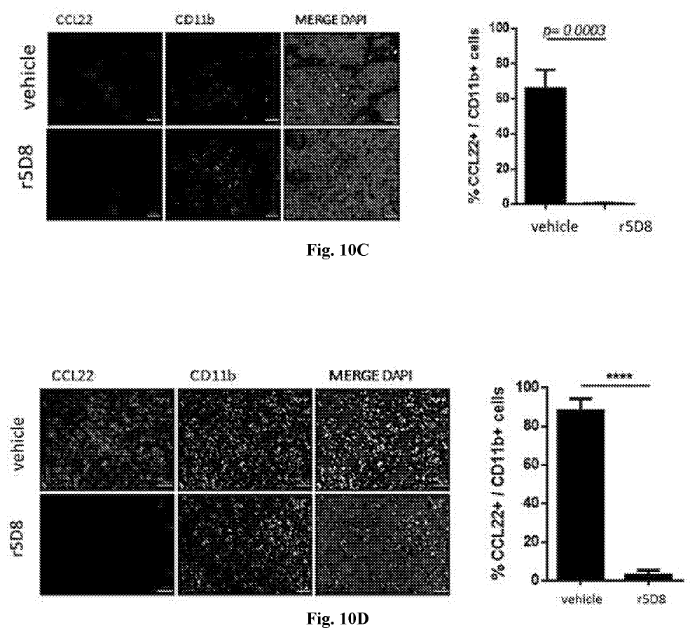

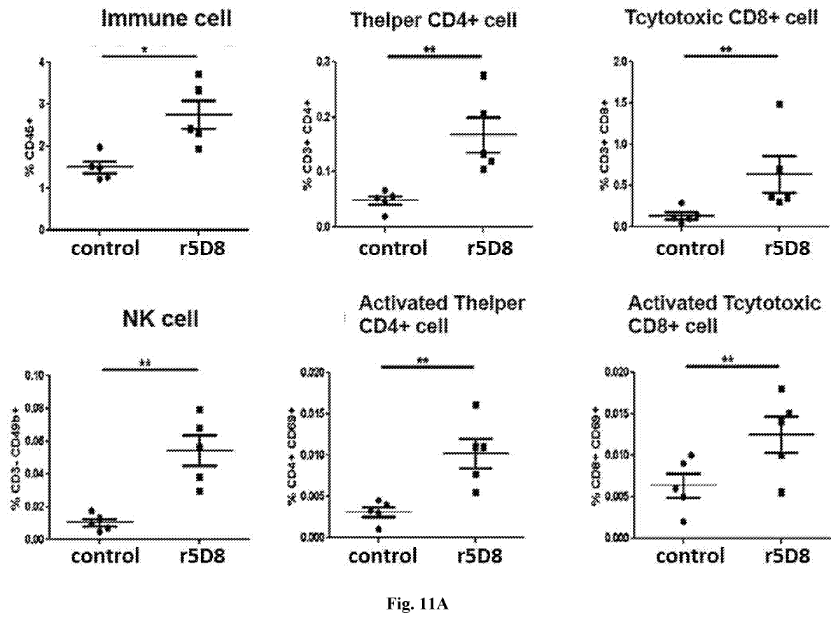

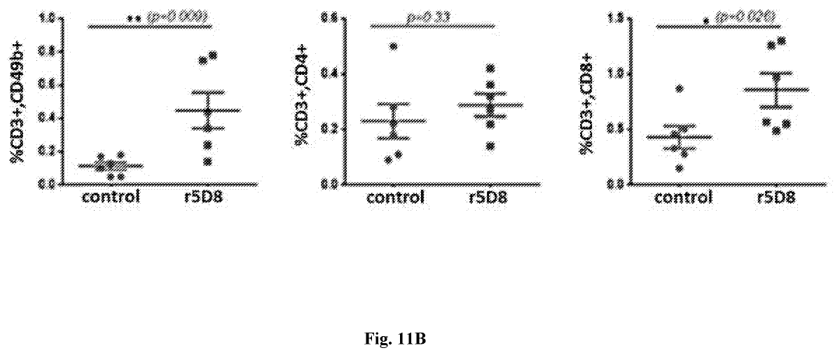

In the U251 GBM orthotopic model, expression of CCL22, a marker of M2 polarized macrophages, was significantly decreased in tumors treated with r5D8 as shown in FIG. 10A. This finding was also confirmed in a physiologically relevant organotypic tissue slice culture model using r5D8 in which three patient samples showed a significant decrease in CCL22 and CD206 (MRC1) expression (also a marker of M2 macrophages) after treatment, as shown in FIG. 10B (compare upper, control, to lower, treated, for both MRC1 and CCL22). Furthermore, r5D8 also decreased CCL22⁺M2 macrophages in syngeneic ID8 (FIG. 10C) and CT26 (FIG. 10D) tumors in immunocompetent mice.

### Example 15—r5D8 Increases Non-Myeloid Effector Cells

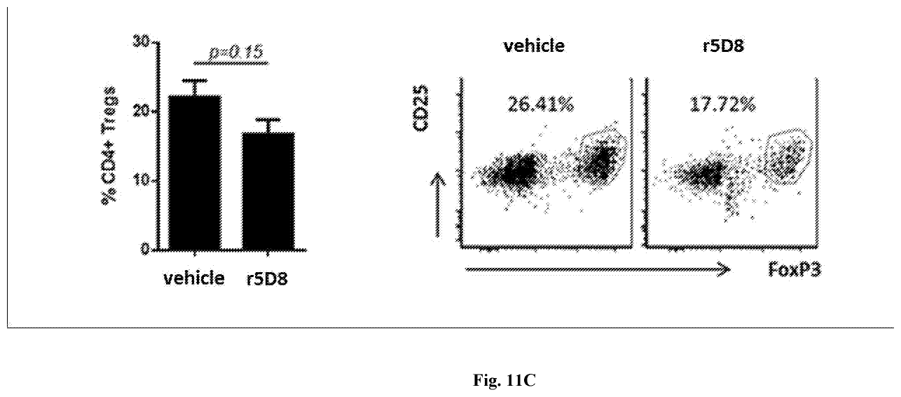

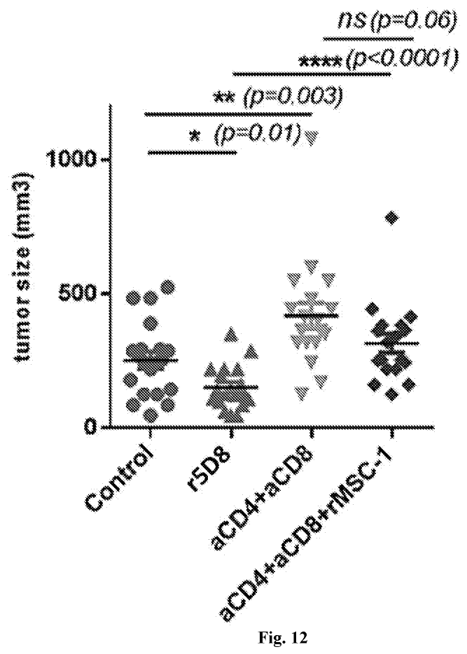

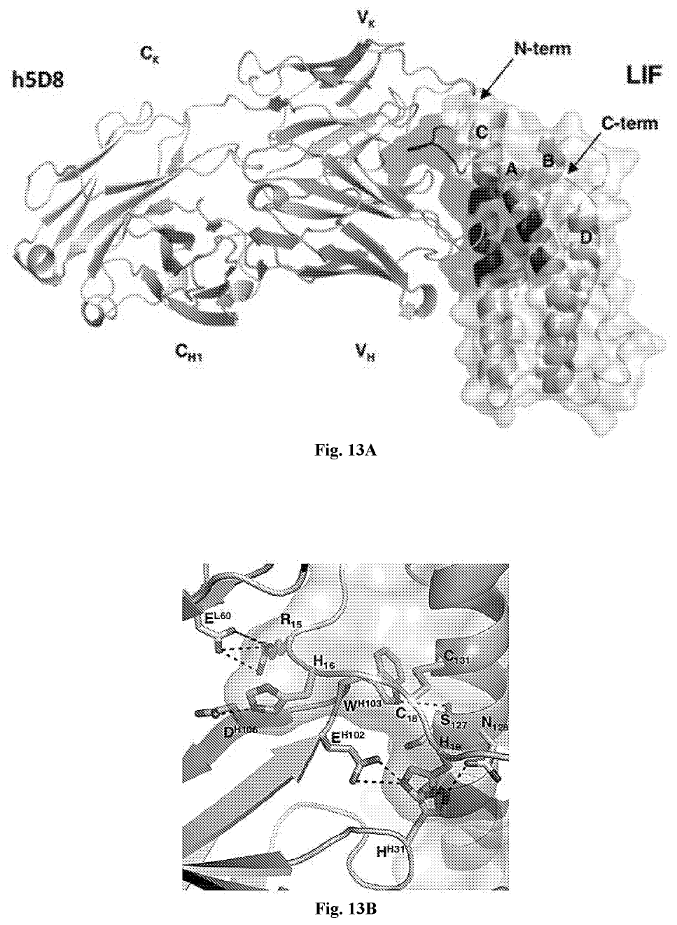

To investigate additional immune mechanisms, the effect of r5D8 on T cells and other non-myeloid immune effector cells within the tumor microenvironment were evaluated. In the ovarian orthotopic ID8 syngeneic model, r5D8 treatment resulted in an increase in intratumoral NK cells and an increase in total and activated CD4⁺ and CD8⁺T cells as shown in FIG. 11A. Similarly, in the colon syngeneic CT26 tumor model, r5D8 increased intratumoral NK cells, increased CD4+ and CD8+T cells and trended to decrease CD4⁺CD25⁺FoxP3⁺T-reg cells as shown in FIG. 11B. A trend for a decrease in CD4⁺CD25⁺FoxP3⁺T-reg cells was also observed in the syngeneic orthotopic KLN205 tumor model following r5D8 treatment as shown in FIG. 11C. Consistent with a requirement for T cells to mediate efficacy, depletion of CD4⁺ and CD8⁺T cells in the CT26 model inhibited the anti-tumor efficacy of r5D8 as shown in FIG. 12.

### Methods for T Cell Depletion

CT26 cells were cultured in RPMI culture medium (Gibco, Invitrogen), supplemented with 10% Fetal Bovine Serum (FBS [Gibco, Invitrogen]), 40 U/mL penicillin and 40 μg/mL streptomycin (PenStrep [Gibco, Invitrogen]) and 0.25 μg/mL Plasmocin (Invivogen). CT26 cells (5×10⁵) were collected, rinsed with PBS, centrifuged at 400 g for 5 minutes and resuspended in 100 μL PBS. Cells were kept on ice to avoid cell death. The CT26 cells were administered in both flanks to mice via subcutaneous injection using a 27G syringe. Mice were treated twice weekly with r5D8 administered intraperitoneally as indicated in the study design. Vehicle control (PBS), rat r5D8, and/or anti-CD4 and anti-CD8 was administered to the mice via intraperitoneal injection (IP) twice weekly as stated in the study design. All antibody treatments were administered concomitantly.

### Example 16-Crystal Structure of h5D8 in Complex with Human LIF

The crystal structure of h5D8 was solved to a resolution of 3.1 angstroms in order to determine the epitope on LIF that h5D8 was bound to and to determine residues of h5D8 that participate in binding. The co-crystal structure revealed that the N-terminal loop of LIF is centrally positioned between the light and heavy chain variable regions of h5D8 (FIG. 13A). In addition, h5D8 interacts with residues on helix A and C of LIF, thereby forming a discontinuous and conformational epitope. Binding is driven by several salt-bridges, H-bonds and Van der Waals interactions (Table 7, FIG. 13B). The h5D8 epitope of LIF spans the region of interaction with gp130. See Boulanger, M. J., Bankovich, A. J., Kortemme, T., Baker, D. & Garcia, K. C. Convergent mechanisms for recognition of divergent cytokines by the shared signaling receptor gp130. Molecular cell 12, 577-589 (2003). The results are summarized below in Table 7 and depicted in FIG. 13.

TABLE 7
Summary of X-Ray crystal structure for h5D8 in complex with
human LIF

| LIF Residue | Interaction | |
|---|---|---|
| (epitope) | type | h5D8 Residue (paratope, Kabat numbering) |
| Ala13 | VDW | L-Tyr49, L-Asn53 |
| Ile14-O | HB | L-Ser50-OG |
| Ile | VDW | L-His30, L-Tyr32, L-Tyr49, L-Ser50 |
| | | H-Trp97 |
| Arg15-NE | SB | L-G1u55-OE1, L-Glu55-OE2 |
| Arg15-NH1 | SB | L-G1u55-OE1, L-Glu55-OE2 |
| Arg15-NH2 | SB | L-G1u55-OE1, L-Glu55-OE2 |
| Arg15-O | HB | L-Asn34-ND2 |
| Arg15 | VDW | L-Asn34, L-Leu46, L-Tyr49, L-G1u55, |
| | | L-Ser56 |
| | | H-G1u96, H-Trp97, H-Asp98, H-Leu99, |
| | | H-Asp101 |
| His16-NE2 | SB | H-Asp101-OD2 |
| His16 | VDW | L-Tyr32, L-Asn34, L-Met89 |
| | | H-Trp95, H-Glu96, H-Trp97, H-Asp101 |
| Pro17 | VDW | L-Tyr32, L-Ala91 |
| | | H-Trp97 |
| Cys18 | VDW | L-Tyr32 |
| | | H-Trp33, H-Trp97 |
| His19-NE2 | SB | H-Glu96-oE1, H-Glu96-OE2 |
| His19 | VDW | H-His31, H-Trp33, H-Glu96 |
| Asn20-OD1 | HB | H-Lys52-NZ |
| Asn20-ND2 | HB | H-Asp53-0D1 |
| Asn20 | VDW | H-Trp33, H-Lys52, H-Asp53 |
| Gln25-NE2 | HB | H-Asp53-OD2 |
| Gln25 | VDW | H-His31, H-Ser52C, H-Asp53 |
| Gln29 | VDW | H-His31 |
| Gln32 | VDW | H-Lys52B |
| Asp120-OD2 | HB | H-Ser30-OG |
| Asp120 | VDW | H-Thr28, H-Ser30 |
| Arg123-NE | HB | H-Thr28-OG |
| Arg123 | VDW | H-Thr28 |
| Gly124 | VDW | H-His31 |
| Leu125 | VDW | H-His31 |
| Ser127-OG | HB | H-Asp98-OD2 |
| Ser127-O | HB | H-Trp97-NE1 |
| Ser127 | VDW | H-His31, H-Trp97, H-Asp98 |
| Asn128-OD1 | HB | H-His31-NE2 |
| Asn128 | VDW | H-His31 |
| Leu130 | VDW | H-Trp97 |
| Cys131 | VDW | H-Trp97 |
| Cys134 | VDW | H-Trp97 |
| Ser135-O | HB | L-His30-NE2 |
| Ser135 | VDW | L-His30 |
| His138 | VDW | L-His30 |

VDW, Van der Waals low energy binding;
HB, hydrogen bond (medium energy binding);
SB, salt bridge (high energy binding)

### Methods

LIF was transiently expressed in HEK 293S (Gnt I^{-/-}) cells and purified using Ni-NTA affinity chromatography, followed by gel-filtration chromatography in 20 mM Tris pH 8.0 and 150 mM NaCl. The recombinant h5D8 Fab was transiently expressed in HEK 293F cells and purified using KappaSelect affinity chromatography, followed by cation exchange chromatography. Purified h5D8 Fab and LIF were mixed at a 1:2.5 molar ratio and incubated at room temperature for 30 min prior to deglycosylation using EndoH. Gel-filtration chromatography was subsequently used to purify the complex. The complex was concentrated to 20 mg/mL and set up for crystallization trials using sparse matrix screens. Crystals formed at 4° C. in a condition containing 19% (v/v) isopropanol, 19% (w/v) PEG 4000, 5% (v/v) glycerol, 0.095 M sodium citrate pH 5.6. The crystal diffracted to a resolution of 3.1 Å at the 08ID-1 beamline at the Canadian Light Source (CLS). Data were collected, processed and scaled using XDS as per Kabsch et al. Xds. Acta crystallographica. Section D, Biological crystallography 66, 125-132 (2010). Structures were determined by molecular replacement using Phaser as per McCoy et al. Phaser crystallographic software. J Appl Crystallogr 40, 658-674 (2007). Several iterations of model building and refinement were performed using Coot and phenix.refine until the structures converged to an acceptable R_{work} and R_{free}. See Emsley et al. Features and development of Coot. Acta crystallographica. Section D, Biological crystallography 66, 486-501 (2010); and Adams, et al. PHENIX: a comprehensive Python-based system for macromolecular structure solution. Acta crystallographica. Section D, Biological crystallography 66, 213-221 (2010) respectively. The figures were generated in PyMOL (The PyMOL Molecular Graphics System, Version 2.0 Schrödinger, LLC).

### Example 17—h5D8 has High Specificity for LIF

The goal was to test binding of h5D8 to other LIF family members to determine the binding specificity. Using Octet96 analysis h5D8 binding to human LIF is approximately 100-fold greater than binding to LIFs highest homology IL-6 family member Oncostatin M (OSM) when both proteins are produced in E. coli. When both proteins are produced in a mammalian system h5D8 exhibits no binding to OSM. Data are summarized in Table 8.

TABLE 8
Summary of h5D8 Affinity Measurements for Cytokines as
Measured by Octet

| | KD [M] | kon [1/1Ms] | kdis [1/s] |
|---|---|---|---|
| h5D8 + huLIF | 4.3E−10 +/− | 3.1E+05 +/− 3.1E+03 | 1.3E−04 +/− |
| (E. coli) | 2.0E−11 | | 5.8E−06 |
| h5D8 + huLIF | 1.3E−09 +/− | 1.2E+05 +/− 1.3E+03 | 1.5E−04 +/− |
| (mammalian) | 7.2E−11 | | 8.5E−06 |
| h5D8 + huOSM | 3.6E−08 +/− | 8.5E+04 +/− 3.1E+03 | 3.1E−03 +/− |
| (E. coli) | 1.4E−09 | | 4.1E−05 |
| h5D8 + huOSM | ND | ND | ND |
| (mammalian) | | | |
| h5D8 + huIL-6 | ND | ND | ND |
| (E. coli) | | | |

ND = no binding

### Methods

Octet Binding Experiments: Reagents were used and prepared as per manufacturer's provided manual. A Basic Kinetics Experiment was performed using Octet Data Acquisition software ver. 9.0.0.26 as follows: Setup of sensors/program: i) Equilibration (60 seconds); ii) Loading (15 seconds); iii) Baseline (60 seconds); iv) Association (180 seconds); and v) Dissociation (600 seconds)

Octet Affinity of h5D8 for cytokines: A Basic Kinetics Experiment was performed using Octet Data Acquisition software ver. 9.0.0.26 as follows: Amine Reactive 2nd Generation Biosensors (AR2G) were hydrated for a minimum of 15 minutes in water. Amine conjugation of h5D8 to the biosensors was performed according to ForteBio Technical Note 26 (please see References) using the Amine Coupling Second Generation Kit. Dip steps were as performed at 30° C., 1000 rpm as follows: i) 60 seconds Equilibration in water; ii) 300 seconds Activation in 20 mM ECD, 10 mM sulfo-NHS in water; iii) 600 second Immobilization of 10 μg/ml h5D8 in 10 mM Sodium Acetate, pH 6.0; iv) 300 seconds Quench in 1M Ethanolamine, pH 8.5; v) 120 seconds Baseline in water. Kinetics experiments were then performed with the following Dip and Read steps at 30° C., 1000 rpm: vi) 60 seconds Baseline in 1× kinetics buffer; vii) 180 seconds Association of appropriate serial dilutions of a cytokine in 1× kinetics buffer; viii) 300 seconds Dissociation in 1× kinetics buffer; ix) Three Regeneration/Neutralization cycles alternating between 10 mM glycine pH 2.0 and 1× kinetics buffer respectively (5 seconds in each for 3 cycles). Following regeneration, the biosensors were reused for subsequent binding analyses.

Human recombinant LIF produced from mammalian cells was from ACROBiosystems (LIF-H521b); human recombinant OSM produced in mammalian cells was from R & D (8475-OM/CF); and human recombinant OSM produced in E. coli cells was from R & D (295-OM-050/CF).

### Example 18—Crystal Structure of h5D8 Fab

Five crystal structures of the h5D8 Fab under a wide spectrum of chemical conditions were determined. The high resolutions of these structures indicate that the conformations of CDR residues are associated with minor flexibility, and are highly similar in different chemical environments. A unique feature of this antibody is the presence of a non-canonical cysteine in position 100 of the variable heavy region. Structure analysis shows that the cysteine is unpaired and largely inaccessible to the solvent.

H5D8 Fab was obtained by papain digestion of its IgG, followed by purification using standard affinity, ion exchange and size chromatography techniques. Crystals were obtained using vapor diffusion methods and allowed to determine five crystal structures ranging between 1.65 Å to 2.0 Å in resolution. All structures were solved in the same crystallographic space group and with similar unit cell dimensions (P212121, a˜53.8 Å, b˜66.5 Å, c˜143.3 Å), despite crystallization conditions ranging across five different pH levels: 5.6, 6.0, 6.5, 7.5 and 8.5. As such, these crystal structures allow for comparison of the three-dimensional disposition of h5D8 Fab unimpeded by crystal packing artefacts and across a wide spectrum of chemical conditions.

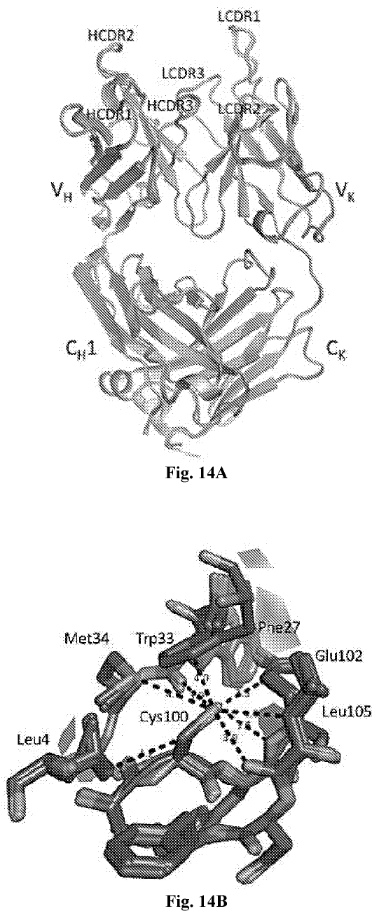

Electron density was observed for all complementarity determining region (CDR) residues, which were subsequently modeled. Noticeably, LCDR1 and HCDR2 adopted elongated conformations that together with shallow LCDR3 and HCDR3 regions formed a binding groove at the center of the paratope (FIG. 14A). The five structures were highly similar across all residues, with all-atoms root mean square deviations ranging between 0.197 Å and 0.327 Å (FIG. 14A). These results indicated that the conformations of CDR residues were maintained in various chemical environments, including pH levels ranging between 5.6 and 8.5 and ionic strengths ranging between 150 mM and 1 M. Analysis of the electrostatic surface of the h5D8 paratope revealed that positively and negatively charged regions equally contributed to hydrophilic properties, with no prevalent hydrophobic patches. h5D8 has the uncommon feature of a non-canonical cysteine at the base of HCDR3 (Cys100). In all five structures, this free cysteine is ordered and does not form any disulfide scrambles. Additionally, it is not modified by the addition of Cys (cysteinylation) or glutathione (glutathiolation) and makes Van der Waals interactions (3.5-4.3 Å distances) with main chain and side chain atoms of Leu4, Phe27, Trp33, Met34, Glu102 and Leu105 of the heavy chain (FIG. 14B). Finally, Cys100 is a predominantly buried structural residue that appears to be involved in mediating the conformations of CDR1 and HCDR3. It is thus unlikely to have reactivity with other cysteines, as observed by a homogeneous disposition of this region in five crystal structures.

### Methods

H5D8-1 IgG was obtained from Catalent Biologics and was formulated in 25 mM histidine, 6% sucrose, 0.01% polysorbate 80, at pH 6.0. The formulated IgG was extensively buffer-exchanged into PBS using a 10K MWCO concentrator (Millipore) prior to digestion with 1:100 microgram papain (Sigma) for 1 hour at 37° C. in PBS, 1.25 mM EDTA, 10 mM cysteine. The papain-digested IgG was flown through a Protein A column (GE Healthcare) using an AKTA Start chromatography system (GE Healthcare). The Protein A flow-through, which contained the h5D8 Fab was recovered and buffer-exchanged into 20 mM sodium acetate, pH 5.6 using a 10K MWCO concentrator (Millipore). The resulting sample was loaded onto a Mono S cation exchange column (GE Healthcare) using an AKTA Pure chromatography system (GE Healthcare). Elution with a gradient of 1 M potassium chloride resulted in a predominant h5D8 Fab peak that was recovered, concentrated and purified to size homogeneity using a Superdex 200 Increase gel filtration column (GE Healthcare) in 20 mM Tris-HCl, 150 mM sodium chloride, at pH 8.0. The high purity of the h5D8 Fab was confirmed by SDS-PAGE under reducing and non-reducing conditions.

Purified h5D8 Fab was concentrated to 25 mg/mL using a 10K MWCO concentrator (Millipore). An Oryx 4 dispenser (Douglas Instruments) was used to set up vapor diffusion crystallization experiments with sparse matrix 96-conditions commercial screens JCSG TOP96 (Rigaku Reagents) and MCSG-1 (Anatrace) at 20° C. Crystals were obtained and harvested after four days in the following five crystallization conditions: 1) 0.085 M sodium citrate, 25.5% (w/v) PEG 4000, 0.17 M ammonium acetate, 15% (v/v) glycerol, pH 5.6; 2) 0.1 M MES, 20% (w/v) PEG 6000, 1 M lithium chloride, pH 6.0; 3) 0.1 M MES, 20% (w/v) PEG 4000, 0.6 M sodium chloride, pH 6.5; 4) 0.085 M sodium HEPES, 17% (w/v) PEG 4000, 8.5% (v/v) 2-propanol, 15% (v/v) glycerol, pH 7.5; and 5) 0.08 M Tris, 24% (w/v) PEG 4000, 0.16 M magnesium chloride, 20% (v/v) glycerol, pH 8.5. Prior to flash-freezing in liquid nitrogen, mother liquors containing the crystals were supplemented with 5-15% (v/v) glycerol or 10% (v/v) ethylene glycol, as required. Crystals were subjected to X-ray synchrotron radiation at the Advanced Photon Source, beamline 23-ID-D (Chicago, Ill.) and diffraction patterns were recorded on a Pilatus3 6M detector. Data were processed using XDS and structures were determined by molecular replacement using Phaser. Refinement was carried out in PHENIX with iterative model building in Coot. Figures were generated in PyMOL. All software were accessed through SBGrid.

### Example 19—Mutations at Cysteine 100 of h5D8 Preserve Binding

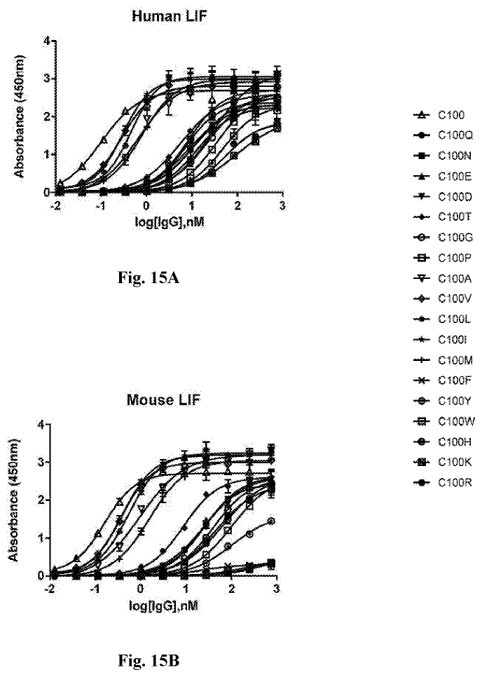

Analysis of h5D8 revealed a free cysteine residue at position 100 (C100) in the variable region of the heavy chain. H5D8 variants were generated by substituting C100 with each naturally occurring amino acid in order to characterize binding to and affinity for human and mouse LIF. Binding was characterized using ELISA and Octet assay. Results are summarized in Table 9. ELISA EC50 curves are shown in FIG. 15 (FIG. 15A human LIF and FIG. 15B Mouse LIF).

TABLE 9
Summary of affinities determined by Octet assay and EC50 determined by ELISA

| | Affinity/k_{D} (M) | | Binding EC50 (nM) | |
|---|---|---|---|---|
| Mutation | human LIF | mouse LIF | human LIF | mouse LIF |
| C100 | <1.0E−12 ± 2.252E−11 | 9.946E−11 ± 8.272E−12 | 0.09878 | 0.1605 |
| C100S | 8.311E−10 ± 5.886E−11 | 2.793E−09 ± 5.925E−11 | n.d. | n.d. |
| C100Q | 3.87E−09 ± 1.55E−10 | 2.84E−09 ± 4.85E−11 | 10.18 | 26.33 |
| C100N | 5.59E−09 ± 1.01E−10 | 6.68E−09 ± 9.8E−11 | 13.18 | 45.87 |
| C100E | 2.67E−09 ± 4.64E−11 | 4.1E−09 ± 7.56E−11 | 7.179 | 25.3 |
| C100D | 2.02E−09 ± 8.08E−11 | 6.49E−09 ± 7.16E−11 | 11.89 | 22.88 |
| C100T | 4.36E−10 ± 2.1E−11 | 1.02E−09 ± 1.77E−11 | 5.575 | 8.753 |
| C100G | 2.49E−09 ± 4.2E−11 | 3.33E−09 ± 5.42E−11 | 21.94 | 40.17 |
| C100P | 2.74E−10 ± 2.97E−10 | <1.0E−12 ± 7.64E−10 | 34.44 | 101.9 |
| C100A | <1.0E−12 ± 2.713E−11 | <1.0E−12 ± 1.512E−11 | 0.6705 | 0.9532 |
| C100V | <1.0E−12 ± 1.805E−11 | <1.0E−12 ± 8.086E−12 | 0.2785 | 0.3647 |
| C100L | <1.0E−12 ± 1.963E−11 | 1.998E−10 ± 1.055E−11 | 0.454 | 0.547 |
| C100I | <1.0E−12 ± 1.424E−11 | 3.361E−11 ± 7.545E−12 | 0.299 | 0.3916 |
| C100M | 1.155E−09 ± 3.400E−11 | 2.676E−09 ± 2.449E−11 | 0.7852 | 1.563 |
| C100F | 4.376E−09 ± 1.127E−10 | 1.147E−08 ± 9.099E−11 | 8.932 | 21.53 |
| C100Y | 1.444E−08 ± 1.159E−09 | 2.514E−08 ± 2.047E−09 | n.d. | n.d. |
| C100W | 2.508E−08 ± 7.036E−09 | 4.819E−08 ± 4.388E−09 | n.d. | n.d. |
| C100H | 1.304E−10 ± 1.416E−10 | 4.284E−09 ± 1.231E−10 | 8.254 | n.d. |
| C100K | 7.477E−08 ± 1.581E−09 | 6.053E−08 ± 2.589E−09 | n.d. | n.d. |
| C100 R | 1.455E−07 ± 6.964E−09 | 5.142E−08 ± 3.247E−09 | n.d. | n.d. |

### Methods

ELISA: Binding of h5D8 C100 variants to human and mouse LIF was determined by ELISA. Recombinant human or mouse LIF protein was coated on Maxisorp 384-well plates at 1 ug/mL overnight at 4° C. Plates were blocked with 1× blocking buffer for 2 hours at room temperature. Titrations of each h5D8 C100 variants were added and allowed to bind for 1 hour at room temperature. Plates were washed three times with PBS+0.05% Tween-20. HRP-conjugated anti-human IgG was added and allowed to bind for 30 min at room temperature. Plates were washed three times with PBS+0.05% Tween-20 and developed using 1×TMB substrate. The reaction was stopped with 1M HCl and absorbance at 450 nm was measured. Generation of figures and non-linear regression analysis was performed using Graphpad Prism.

Octet RED96: The affinity of h5D8 C100 variants to human and mouse LIF was determined by BLI using the Octet RED96 system. h5D8 C100 variants were loaded onto Anti-Human Fc biosensors at 7.5 ug/mL following a 30 second baseline in 1× kinetics buffer. Titrations of human or mouse LIF protein were associated to the loaded biosensors for 90 seconds and allowed to dissociate in 1× kinetics buffer for 300 seconds. KDs were calculated by the data analysis software using a 1:1 global fit model.

### Example 20—h5D8 Blocks Binding of LIF to Gp130 In Vitro

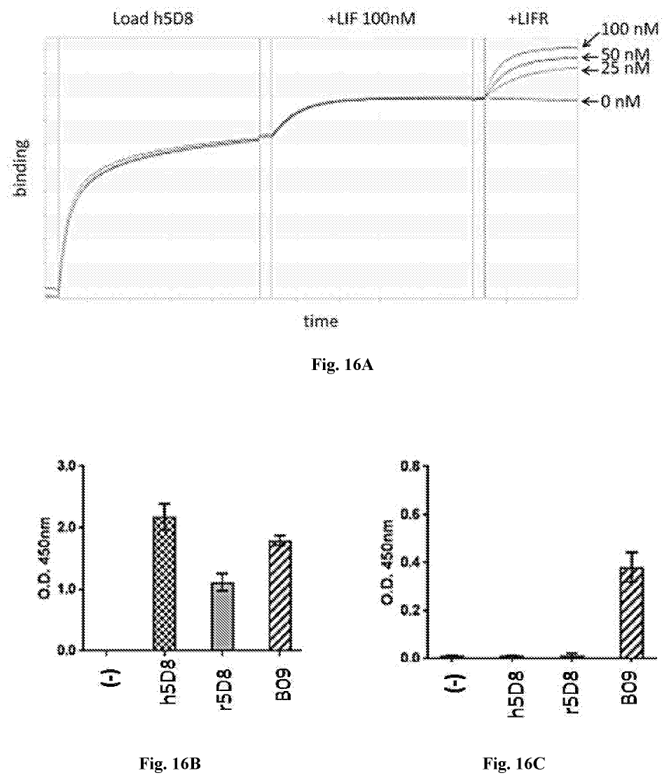

To determine whether h5D8 prevented LIF from binding to LIFR, a molecular binding assay using the Octet RED 96 platform was performed. H5D8 was loaded onto AHC biosensors by anti-human Fc capture. Then, the biosensors were dipped in LIF and, as expected, association was observed (FIG. 16A, middle third). Subsequently, the biosensors were dipped in different concentrations of LIFR. A dose-dependent association was observed (FIG. 16A, right third). The control experiment demonstrated that this association was LIF-specific (not shown), and not due to a non-specific interaction of LIFR with h5D8 or with the biosensors.

To further characterize the binding of h5D8 and LIF, a series of ELISA binding experiments was conducted. H5D8 and LIF were pre-incubated and were then introduced to plates coated with either recombinant human LIFR (hLIFR) or gp130. The lack of binding between the h5D8/LIF complex and the coated substrate would indicate that h5D8 in some way disrupted the binding of LIF to the receptor. Additionally, control antibodies that either did not bind LIF (isotype control, indicated by (−)) or that bind LIF at known binding sites (B09 does not compete with either gp130 or LIFR for LIF binding; r5D8 is the rat parental version of h5D8) were also used. The ELISA results demonstrated that the h5D8/LIF complex was able to bind hLIFR (as was r5D8/LIF complex), indicating that these antibodies did not prevent the LIF/LIFR association (FIG. 16A). In contrast, the h5D8/LIF complex (and a r5D8/LIF complex) was not able to bind recombinant human gp130 (FIG. 16B). This indicates that the gp130 binding site of LIF was affected when LIF was bound to h5D8.

### Example 21—LIF and LIFR Expression in Human Tissues

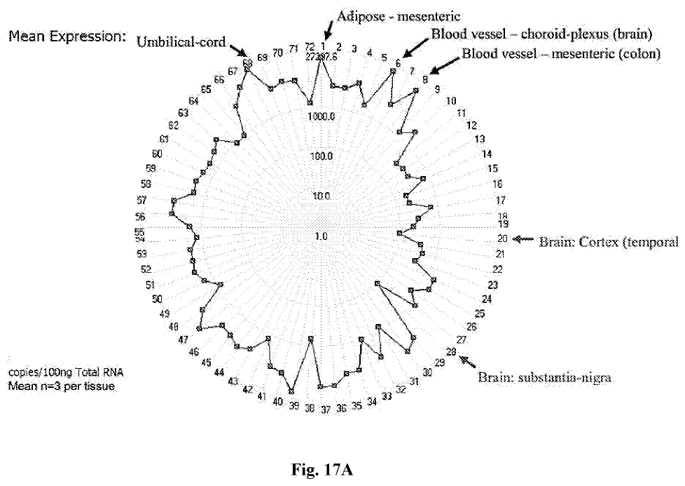

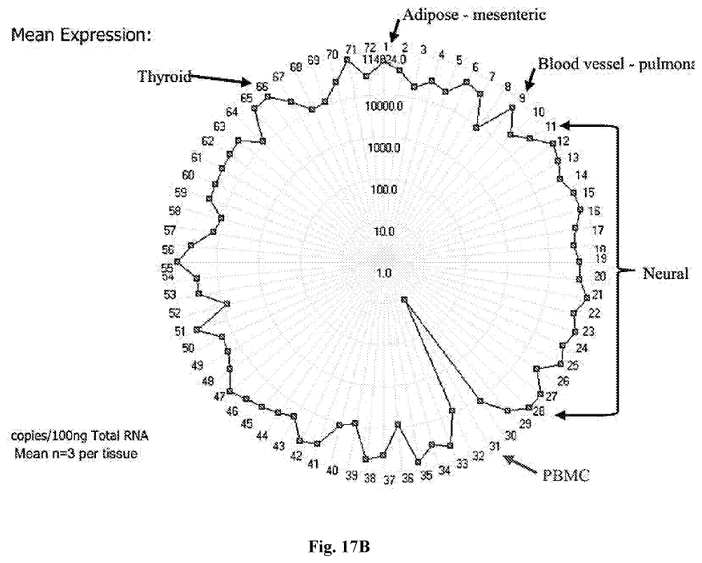

Quantitative real-time PCR was performed on many different types of human tissue in order to determine expression levels of LIF and LIFR. The mean expression levels shown in FIGS. 17A and 17B are given as copies per 100 ng of total RNA. Most tissues expressed at least 100 copies per 100 ng of total RNA. LIF mRNA expression was highest in human adipose tissue (mesenteric-ileum [1]), blood-vessel tissue (choroid-plexus [6] and mesenteric [8]) and umbilical cord [68] tissue and lowest in brain tissue (cortex [20] and substantia-nigra [28]). LIFR mRNA expression was highest in human adipose tissue (mesenteric-ileum [1]), blood vessel tissue (pulmonary [9]), brain tissue [11-28] and thyroid [66] tissue and was lowest in PBMCs [31]. LIF and LIFR mRNA expression levels in cynomolgus tissues were similar to those observed in human tissues, wherein LIF expression was high in adipose tissue and LIFR expression was high in adipose tissue and low in PBMCs (data not shown).

The tissue numbering for FIG. 17A and FIG. 17B is: 1—adipose (mesenteric-ileum); 2—adrenal gland; 3—bladder; 4—bladder (trigone); 5—blood-vessel (cerebral: middle-cerebral-artery); 6—blood vessel (choroid-plexus); 7—blood vessel (coronary artery); 8—blood vessel (mesenteric (colon)); 9—blood vessel (pulmonary); 10—blood vessel (renal); 11—brain (amygdala); 12—brain (caudate); 13—brain (cerebellum); 14 brain—(cortex: cingulate-anterior); 15—brain (cortex: cingulate-posterior); 16—brain (cortex: frontal-lateral); 17—brain (cortex: frontal-medial); 18—brain (cortex: occipital); 19—brain (cortex: parietal); 20—brain (cortex: temporal); 21—brain (dorsal-raphe-nucleus); 22—brain (hippocampus); 23—brain (hypothalamus: anterior); 24—brain (hypothalamus: posterior); 25—brain (locus coeruleus); 26—brain (medulla oblongata); 27—brain (nucleus accumbens); 28—brain (substantia nigra); 29—breast; 30—caecum; 31—peripheral blood mononuclear cell (PBMCs); 32—colon; 33—dorsal root ganlia (DRG); 34—duodenum; 35—fallopian tube; 36—gallbladder; 37—heart (left atrium); 38—heart (left ventricle); 39—ileum; 40—jejunum; 41—kidney (cortex); 42—kidney (medulla); 43—kidney (pelvis); 44—liver (parenchyma); 45—liver (bronchus: primary); 46—liver (bronchus: tertiary); 47—lung (parenchyma); 48—lymph gland (tonsil); 49—muscle (skeletal); 50—esophagus; 51—ovary; 52—pancreas; 53—pineal gland; 54—pituitary gland; 55—placenta; 56—prostate; 57—rectum; 58—skin (foreskin); 69—spinal cord; 60—spleen (parenchyma); 61—stomach (antrum); 62—stomach (body); 63—stomach (fundus); 64—stomach (pyloric canal); 65—testis; 66—thyroid gland; 67—trachea; 68—umbilical cord; 69—ureter; 70—uterus (cervix); 71—uterus (myometrium); and 72—vas deferens.

### Example 22-Dose Selection, Dose Increments and Flat Dosing

Anti-LIF antibody dose selection, dose increments and flat dosing are described below. Mice and cynomolgus monkeys were used for the safety evaluation of h5D8.

No treatment-related adverse effects were observed in 4-week GLP toxicity studies in mice and monkeys which received weekly IV dosing up to 100 mg/kg. Thus, the highest non-severely toxic dose (HNSTD) is >100 mg/kg and the no-observed-adverse-effect-level (NOAEL) was established as 100 mg/kg IV in both species under the conditions of the studies. The dosage was scaled to establish a human equivalent dose (HED). A body surface area (BSA)-based scaling approach was adopted for the estimation of the HED Based on these GLP toxicology studies a maximum recommended starting dose (MRSD) was estimated as shown below:

0.81 mg/kg IV HED from mouse NOAEL with 10-fold safety factor

>10 mg/kg IV based on 1/10 the severely toxic dose in mice

3.2 mg/kg IV HED from cynomolgus monkey NOAEL with 10-fold safety factor

>16.7 mg/kg IV based on ⅙ the HNSTD

Based on the toxicology studies, and taking a conservative approach for an advanced cancer patient population in the Phase 1 study, a MRSD of 1 mg/kg (or 75 mg flat dose) IV was supported by the data.

The pharmacologically active dose (PAD) has also been considered in setting the MRSD. Based on pharmacology, PK and LIF stabilization data in mouse pharmacology models available to date, the following approach was used to estimate the PAD. Based on the dose-response in the U251 mouse xenograft model, the optimal efficacious dose was considered to be about 300 μg IP twice weekly; this dose level was associated with a trough serum level before the last dose of about 230 μg/mL. There was evidence that maximal stabilization of serum LIF levels had been achieved at this 300 μg dose in this model, which was also supported by serum LIF stabilization data in the mouse GLP toxicity study at doses of 10, 30 and 100 mg/kg. Using a PK model based on a 2-compartmental model fitted to the monkey PK data and scaled for humans, a clinical dose of 1500 mg every 3 weeks would provide a C_{trough} of about 500μg/mL. Similarly, the minimally effective dose of 20 μg twice weekly in this U251 mouse xenograft model was associated with a trough serum level before the last dose of about 20 μg/mL; there was evidence that only about 50% of maximal serum LIF stabilization was achieved at this 20-μg dose, supported by evidence of minimal LIF stabilization at a dose of 0.5 mg/kg IV in the mouse PK-tolerability study. A clinical dose of 75 mg every 3 weeks would provide a C_{trough} of about 25 μg/mL. Additional PK-PD (LIF stabilization) data available from mouse syngeneic models supported the PAD derived from the U251 mouse xenograft model.

Thus, a starting dose of 75 mg i.v. was considered appropriate based on both the toxicology data in mice and monkeys and the minimal effective dose in a mouse xenograft model. A maximum clinical dose of 1500 to 2000 mg was supported by the toxicology data. A flat-dosing approach was appropriate based on the observation of a linear PK in animal models, in conjunction with the absence of test-article related adverse findings.

### Example 23-LIF Expression in Different Cancers

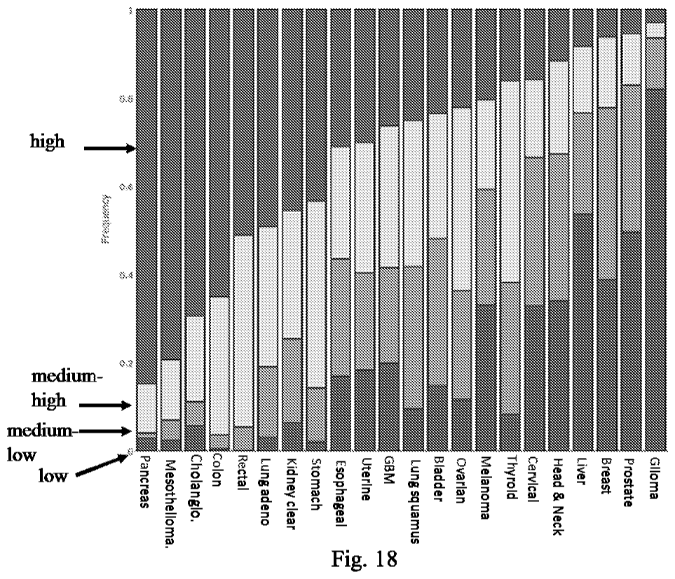

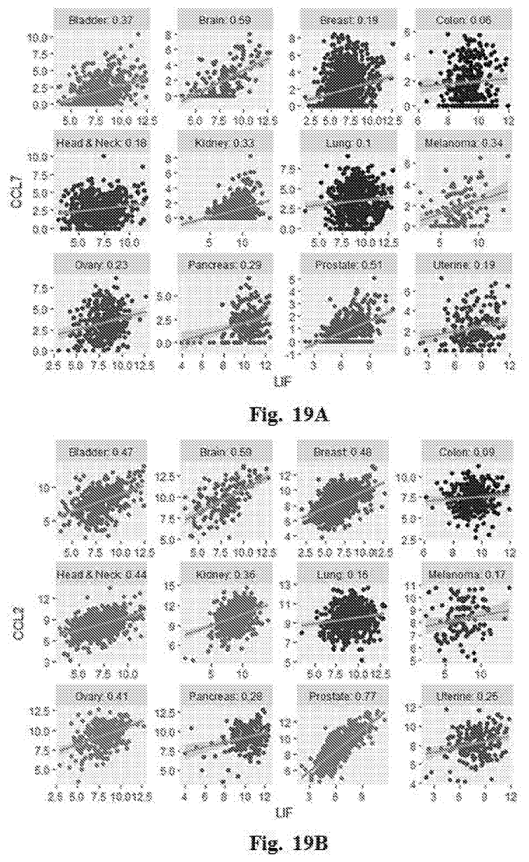

Setting treatment based on LIF levels regardless of cancer type would be a feasible method if there is heterogeneity of LIF expression in different cancer types. FIG. 18 shows that certain cancers such have a higher frequency of high LIF mRNA levels. Even cancers that have a relatively high frequency of low-LIF expression have a subset of individuals that would benefit from anti-LIF treatment.

### Method

RNA sequencing data was obtained from The Cancer Genome Atlas repository for 7,769 samples across 22 indications. LIF transcript expression was thresholded into high, medium-high, medium-low, and low based on top, upper middle, lower middle, and lowest quartile of LIF expression calculated across all samples.

### Example 24-LIF Expression Correlates with T Regulatory Chemokines in Different Cancers

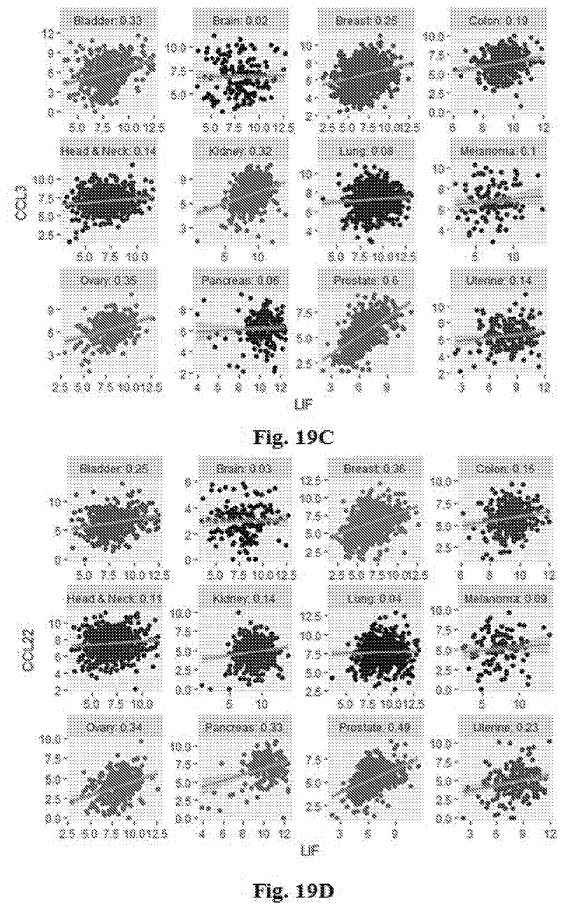

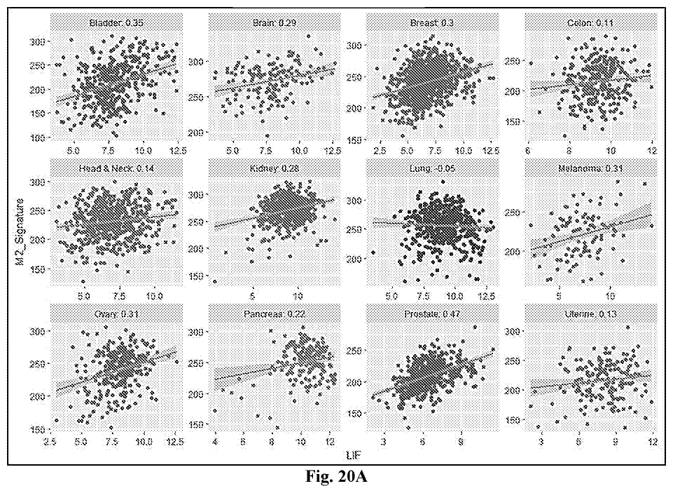

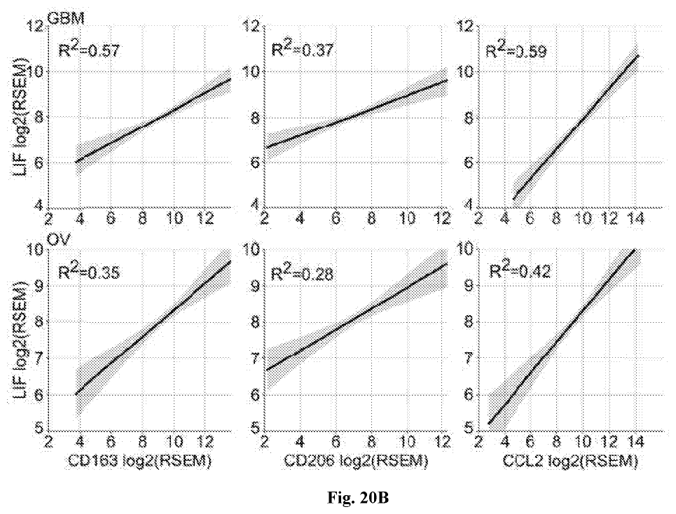

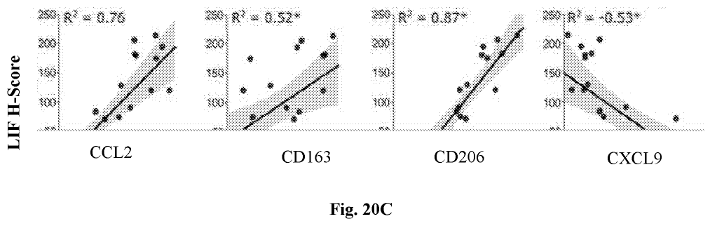

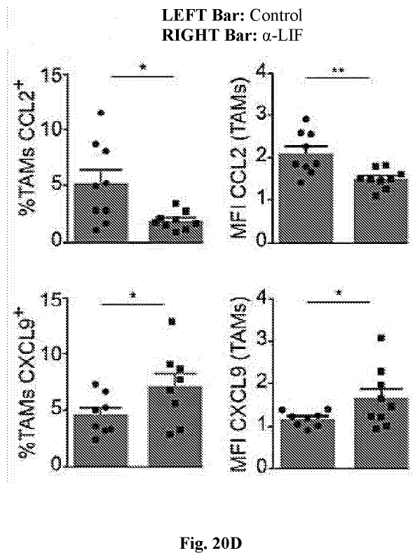

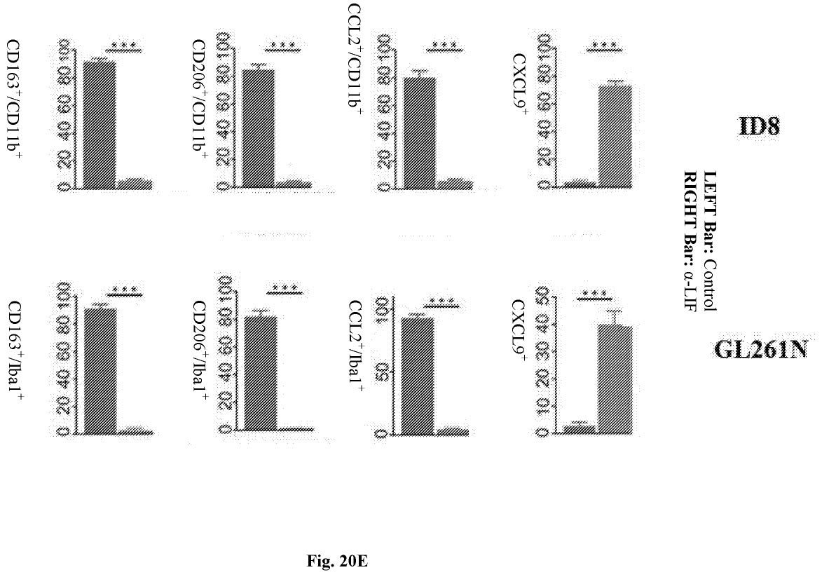

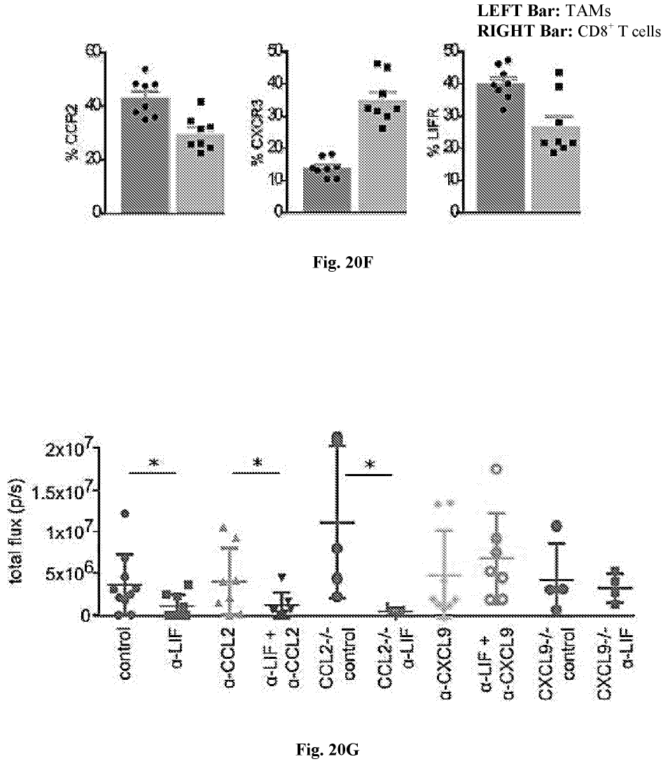

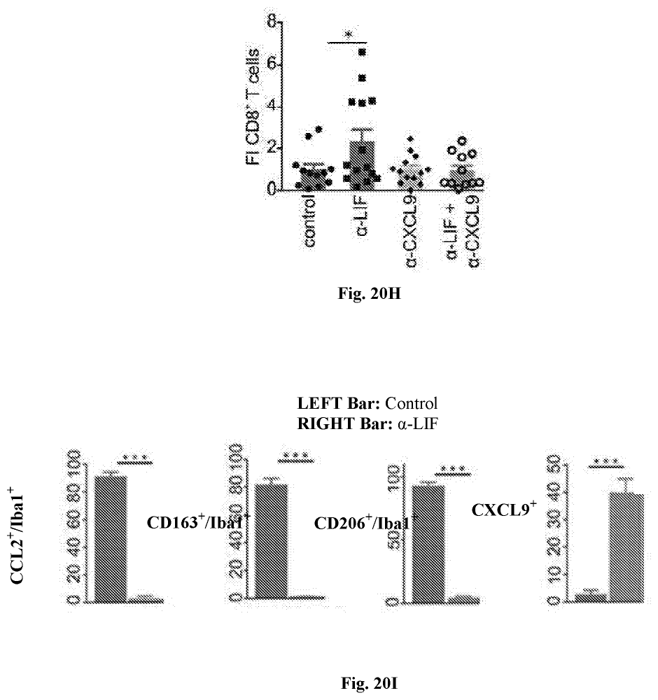

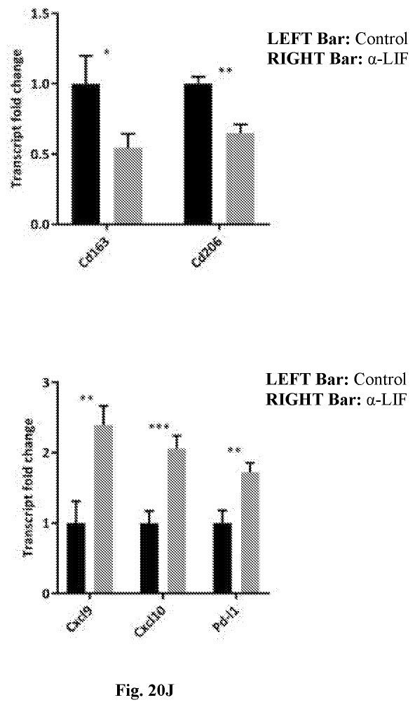

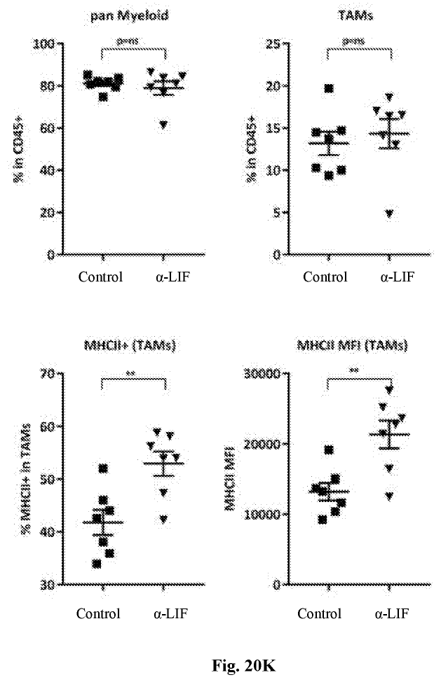

As noted previously LIF inhibition has the effect of reducing immunosuppressive macrophage populations (e.g., M2 macrophages) in mouse and human ex vivo models (FIGS. 10A-10D) and of reducing regulatory CD4+ T cells in a mouse model of NSCLC (FIG. 11C) and infiltration. Thus, cancers that have a high level of both LIF and T regulatory chemokines or secreted by myeloid cells or M2 macrophages can define a subset of individuals with a high likelihood of responding to anti-LIF treatment. T regulatory cells are immunosuppressive CD4+ cells and M2 macrophages are macrophages that support an anti-inflammatory immunosuppressive environment in tissues, as opposed to M1 macrophages which support a pro-inflammatory environment. FIG. 19A to 19D shows correlation of expression of LIF mRNA and the mRNA of different T regulatory chemokines CCL7 (FIG. 19A), CCL2 (FIG. 19B), CCL3 (FIG. 19A), and CCL22 (FIG. 19A). Likewise, FIG. 20A shows correlation of expression of LIF mRNA and the mRNA defining an M2 macrophage signature. These provide basis to segment the population of individuals with LIF expression (even those with low or medium-low LIF) as potential responders to an anti-LIF therapeutic antibody if an immune suppressive signature is present (e.g., based on expression of M2 markers, T regulatory chemokines, or T regulatory cells).

A significantly positive correlation between LIF and CCL2, CD163, and CD206 was seen in both the analysis of TCGA datasets of human GBM and ovarian cancer (FIG. 20B). No correlation was observed between LIF and CXCL9 (data not shown) but relatively low levels of CXCL9 mRNA were observed across tumors. These results were validated at the protein level, by analyzing a cohort of 20 GBM patients and performing LIF, CXCL9, CCL2, CD163, and CD206 IHC of the tumors. A strong positive correlation between LIF and CCL2, CD163, and CD206 was observed (FIG. 20C). CXCL9 was expressed in isolated clusters of cells explaining the low levels of CXCL9 mRNA present in tumors. Notably, CXCL9 showed an inversed correlation with LIF in human GBM (FIG. 20C).

CXCL9 and CCL2 stood out as chemokines critical for CD8⁺ T cell tumor infiltration, and the recruitment of TAMs and Tregs, respectively. CXCL9 and CCL2 regulation by the neutralization of LIF in TAMs (CD11b⁺ Ly6G⁻ Ly6C⁻) was confirmed (FIG. 20D). Immunostaining and isolation of TAMs showed that CXCL9, CCL2, CD206, and CD163 were mainly expressed in TAMs (FIG. 20E) and treatment with anti-LIF (h5D8) regulated their expression (FIG. 20D, 20E). CXCR3 (CXCL9 receptor), CCR2 (CCL2 receptor), and LIFR were expressed in TAMs and CD8⁺ T cells (FIG. 20F).

CXCL9 and CCL2 knockout (CXCL9^{-/-}, CCL2^{-/-}) mouse models were used to test for the relevance of the regulation of CXCL9 and CCL2 in the LIF oncogenic function. Tumors in these mouse models were treated with blocking antibodies against CXCL9 and CCL2. Interestingly, the anti-tumor response to the inhibition of LIF was blunted in the CXCL9^{-/-} mice but not in the CCL2^{-/-} mice (FIG. 20G). Similarly, the CXCL9 neutralizing antibody but not the CCL2 antibody impaired the anti-cancer response to anti-LIF (h5D8) (FIG. 20G). These results indicated that the main mediator of the anti-LIF (h5D8) response was CXCL9. As expected, the blockade of CXCL9 decreased CD8⁺ T cell tumor infiltration in response to anti-LIF (h5D8) (FIG. 20H).

To confirm that LIF regulates immune cell tumor infiltration through the repression of CXCL9 in tumors from actual cancer patients, organotypic tissue cultures were generated from GBM specimens freshly obtained from patients. These organotypic models allow for the short-term culture of slices of tumors that maintain the tissue architecture and stroma (including immune cells) of the tumor of the patient. Organotypic tissue cultures from 3 patients whose tumor cells expressed high levels of LIF (FIG. 20I). In all 3 cultures a large infiltration of TAMs was present as detected by the Iba1 marker and most of the TAMs expressed CCL2, CD163, and CD206. Interestingly, a 3-day treatment of the organotypic culture with a neutralizing antibody against LIF promoted a decrease in CCL2, CD163, and CD206 and an increase in CXCL9 expression (FIG. 20I).

Similar to the above observation in anti-LIF (h5D8) treated human macrophages, anti-LIF (h5D8) treatment was observed to induce down-regulation of the M2 markers CD206 and CD163 in CT26 tumors (FIG. 20J). In contrast, anti-LIF (h5D8) treatment induced increases in expression of immune-stimulatory M1 markers including CXCL9, CXCL10, and PD-L1 (FIG. 20J). These findings were further extended by also examining TAM phenotypes in anti-LIF (h5D8) treated MC38 tumors. Whereas treated tumors were observed to show no difference in the overall frequency of total myeloid or TAM populations, anti-LIF (h5D8) treatment induced an increase in both the proportion of TAMs expressing MHCII as well as the overall expression level of MHCII (FIG. 20K). A parallel comparison of M1/M2 skewing across CT26 and MC38 models was not possible as the MC38 TAMs did not express CD206, a key marker used to phenotype M2 macrophages in the CT26 model. Together, these data demonstrate that anti-LIF treatment inhibits tumor growth in two independent pre-clinical tumor models. The analysis demonstrates that anti-LIF treatment affected TAM phenotypes, but not overall numbers of TAMs or total myeloid cells, suggesting that the observed efficacy occurs, likely in part, through the reprogramming of TAMs to favor anti-tumor immunity.

### Methods

RNA sequencing data was obtained from The Cancer Genome Atlas repository. The association between LIF expression and various T regulatory cell chemokines (CCL7, CCL2, CCL3 and CCL22) was calculated based on Pearson correlation for bladder, brain, breast, colon, head & neck, kidney, lung, melanoma, ovary, pancreas, prostate and uterine cancer samples. The association between LIF expression and a transcriptional signature representing M2 macrophages was calculated based on Pearson correlation for bladder, brain, breast, colon, head & neck, kidney, lung, melanoma, ovary, pancreas, prostate and uterine cancer samples.

Cells were lysed for mRNA extraction (RNeasy Mini or Micro Kit, Qiagen), retrotranscription (iScript Reverse Supermix from BioRad for mRNA), and qRT-PCR was performed using Taqman probes from Applied Biosystems, according to manufacturer's recommendations. For paraffin-embedded sections, RNA was obtained by using High Pure FFPET RNA isolation kit (Roche) and following manufacturer instructions. Reactions were carried out in a CFX384 Touch™ Real-Time PCR Detection System (Bio-Rad) and results were expressed as fold change calculated by the Ct method relative to the control sample. Murine or human ACTB or GAPDH were used as internal normalization controls.

RNA was assayed on the Affymetrix microarray platform with the Mouse Gene 2.1 ST. Next, it was normalized based on a Robust-Microarray Average (RMA). The genes identified to be differentially expressed in anti-LIF treated mice through a Bayesian linear regression, considering paired samples, using limma Bioconductor package.

In the mouse experiments, nuclei were counterstained with DAPI and images were captured using a laser scanning confocal NIKON Eclipse Ti microscope. Quantification of immunofluorescence were performed with ImageJ, counting all or up to 100 cells positive for CD11b, Iba1 or CD3 of 2-3 different fields of each mouse, 3-5 mice/group, and calculating the percentage of those cells positive for CCL2, CD206, and CD163 inside the Iba1 (for GL261N model) or CD68/CD11b (for ID8 model) positive population. For CXCL9, it was calculated the percentage of cells surrounded by the signal of this cytokine inside the total population of cells. For organotypic slices, 3-4 fields of each patient (n=3) were quantified. For organotypic tissue immunofluorescence, five different Z-stack images per condition were processed with Fiji-Image J software. For CD8⁺ T cells, percentage of CD8⁺ T cells was calculated among the total population. Data in graphs are represented as mean±SEM.

Immunofluorescence antibodies: human/murine CCL2 (Novus Biologicals, 1:200), human/murine CD11b (AbCam; 1:2000), human/murine Iba1 (Wako; 1:1000), murine CD68 (AbCam; 1:200), human/murine CD206 (Abcam; 1:500), murine CD163 (Abcam; 1:200), CXCL9 (murine Novus Biologicals 1:200; human Thermo Fischer Scientific; 1:200), and human CD8 (DAKO; 1:200).

Human GBM specimens were obtained from the Vall d'Hebron University Hospital and Clinic Hospital. The clinical protocol was approved by the Vall d'Hebron Institutional Review Board and Clinic Hospital (CEIC), with informed consent obtained from all subjects.

GBM organotypic slice cultures were generated as follows. After resection, surgical specimens were cut with a scalpel into rectangular blocks of 5-10 mm length and 1-2 mm width and individually transferred into 0.4 μm membrane culture inserts (Millipore) within 6-well plates. Before placing the inserts into 6-well plates, 1.2 ml of Neurobasal medium (Life Technologies) supplemented with B27 (Life Technologies), penicillin/streptomycin (Life Technologies) and growth factors (20 ng/ml EGF and 20 ng/ml FGF-2) (PeproTech) were placed into each well. The cultures were kept at 37° C. with constant humidity, 95% air and 5% CO². After one day, slices were treated with a rat anti-mouse/human LIF blocking antibody (h5D8) (referred to as anti-LIF) (developed in house) or with its corresponding normal IgG (10 μg/ml) for 3 days. For the blocking CXCL9 studies, a neutralizing mouse monoclonal antibody against human CXCL9 (R&D Systems) was added to the culture at 1.5 μg/ml. In some occasions, 0.1 ng/ml of human rIFNγ (R&D Systems) was added for 24 h. In parallel, peripheral blood mononuclear cells (PBMCs) were obtained from the whole blood of the same patient by centrifuge density separation using Lymphosep (Biowest). PBMCs were cryopreserved in RPMI medium supplemented with 10% inactivated FBS and 10% DMSO until use. For immune cell infiltration assays, control or anti-LIF slices were embedded into Matrigel (Corning) with subsequent addition of 1×10⁶ PBMCs into 24-well plate in complete RPMI medium. In addition, supernatants were collected and organotypic slices were recovered from Matrigel and further processed for IF and flow cytometry. In some conditions, PBMCs were resuspended with PBS at a concentration of 10⁶ cells/ml and incubated for 20 min with 5 μM Cell Trace CFSE (Invitrogen). After the incubation, cells were washed with RPMI and added to the sections embedded into Matrigel. After 24 h, fluorescent PBMCs invasion into Matrigel was evaluated under microscope by counting migrating cells in five different areas per each condition.

Slides were deparaffinized and hydrated. Antigen retrieval was performed using pH 6 or pH 9 Citrate Antigen Retrieval Solution (DAKO), 10 min 10% peroxidase (H2O2) and blocking solution (2% BSA) for 1 h at room temperature. As a detection system, EnVision FLEX+(DAKO) was used according to the manufacturer's instructions, followed by counterstaining with hematoxilin, dehydration and mounting (DPX). The quantification of LIF, CCL2, CD163, CD206, and CXCL9 staining in GBM tumors from patients was expressed as H score (3×percentage of strong staining+2×percentage of moderate staining+percentage of weak staining), giving a range of 0 to 300. Quantification of p-STAT3, Ki67, CC3 and CD8 was performed with ImageJ, counting the total number of cells of three different fields per mouse, five mice/group, and calculating the percentage of positive cells. Data in graphs are presented as mean±SEM.

Immunohistochemical antibodies: human LIF (Atlas; 1:200), murine LIF (AbCam; 1:200), murine p-STAT3 (Cell Signaling; 1:50), murine Ki67 (AbCam; 1:200), murine Cleaved-Caspase3 (CC3) (Cell Signaling; 1:500), murine CD8 (Bioss; 1:200), human/murine CCL2 (Novus Biologicals, 1:200), human CXCL9 (Thermo Fischer Scientific; 1:100) and human CD163 (Leica Novacastra; 1:200).

### Example 25-LIF Induces Type II (M2) Macrophage Polarization

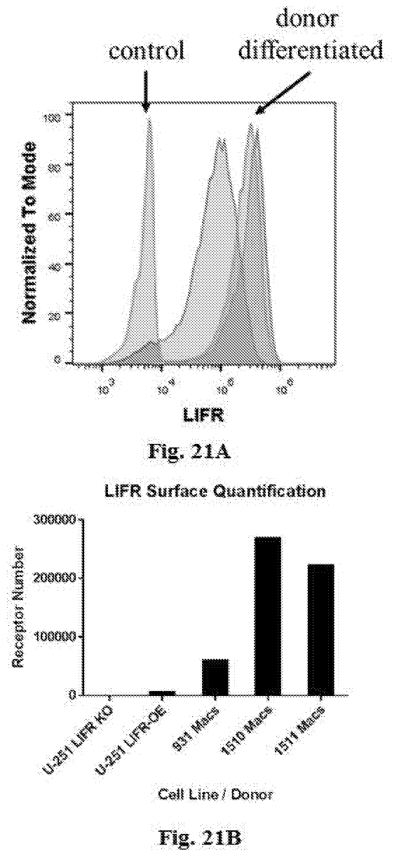

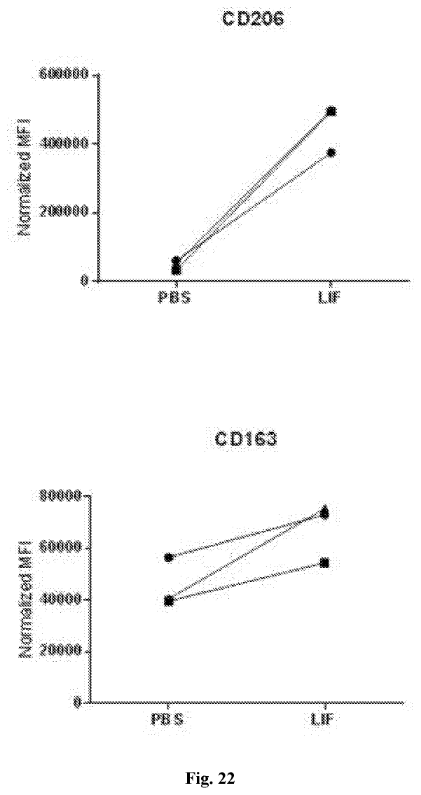

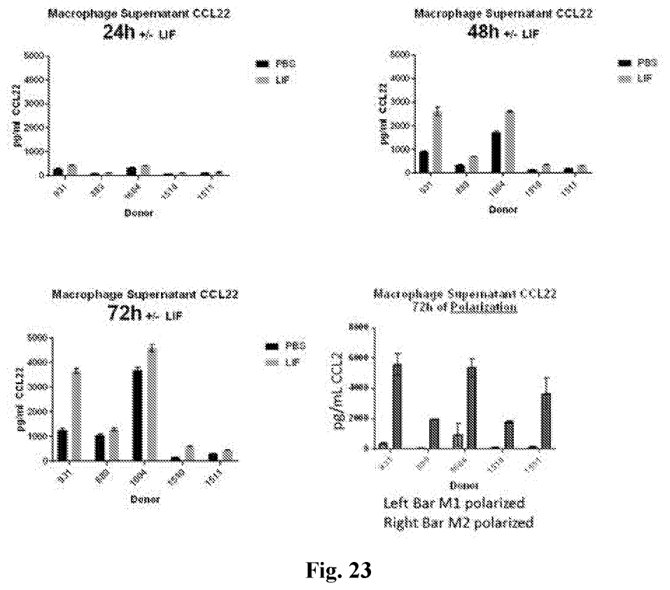

A dual LIF-immunosuppressive signature stratification is potentially a robust way to identify individuals especially prone to respond to an anti-LIF therapeutic treatment. Further evidence for this is shown in FIGS. 21A and 21B, 22, and 23. FIGS. 21A and 21B show that human primary macrophages up regulate LIF receptor after culturing monocytes for 7d with 50 ng/ml M-CSF. Cells from three different individuals are shown. FIGS. 22 and 23 additionally shows that primary human macrophages upregulate macrophage surface markers CD206 and CD163 (FIG. 22), and secretion of CCL22 (FIG. 23), after 72 hours of culture with LIF. Interestingly, CCL22 secretion is much more robust in M2 polarized macrophages (FIG. 23, Lower right). This data provides a mechanistic link between LIF expression and the presence of an immunosuppressive signature indicating that a dual LIF-immunosuppressive signature for selecting patient treatment may apply to all cancer types and in all tissues.

### Method

Macrophages were differentiated from CD14⁺ peripheral human monocytes by culturing in RPMI-1640 media with 10% heat-inactivated FBS, penicillin/streptomycin and 50 ng/ml M-CSF for 7 days. In some experiments, this was followed by culturing in the same media with added 20 nM LIF, or PBS (for control), or 100 ng/ml LPS and 25 ng/ml IFNγ (for M1 polarization), or 20 ng/ml each of IL-4, IL-10 and TGFβ (for M2 polarization) for the additional times indicated.

### Example 26-LIF Receptor Expression on Myeloid Cells from Different Cancers

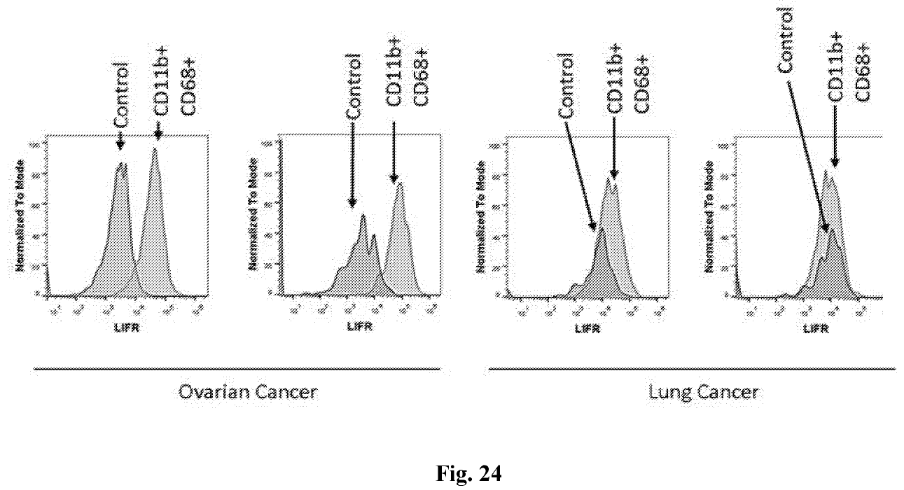

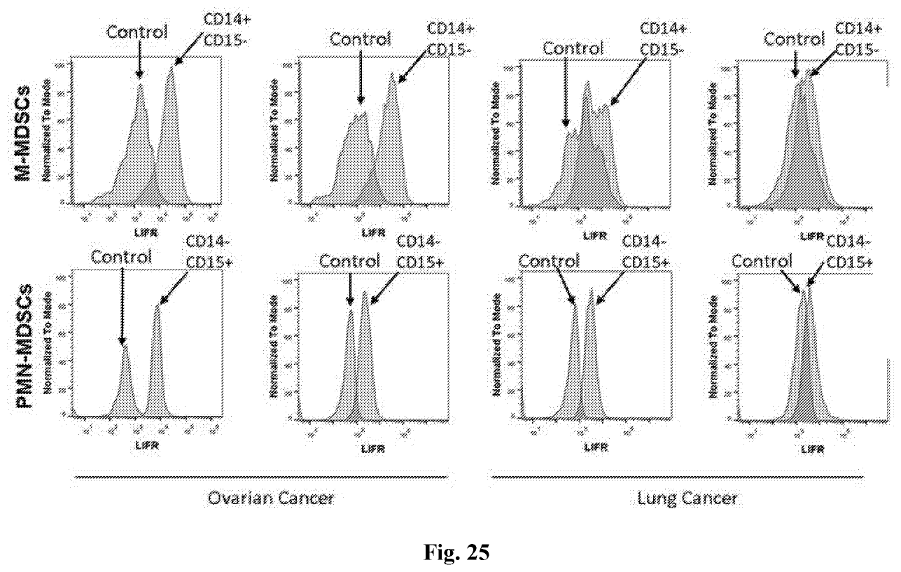

Additional support for the prospect of a dual LIF-immunosuppressive signature as important for tumor growth and survival of cancer is that human tumor associated macrophages, in fact, do express LIF receptor. FIGS. 24 and 25 show that macrophages isolated from 3 of 4 dissociated tumor cells (of ovarian cancer and lung cancer express LIF receptor (FIG. 24). Additionally, LIF receptor is expressed on the cell surface of tumor associated myeloid derived suppressor cells, both monocytic myeloid derived suppressor cells (M-MDSC) and polymorphonuclear myeloid derived suppressor cells (PMN-MDSC) (FIG. 25).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

As used herein, unless otherwise indicated, the term “about” refers to an amount that is near the stated amount by at least 10%.

### Sequences

| SEQ | |
|---|---|
| ID | |
| NO | Sequence |
| 1 | GFTFSHAWMH |
| 2 | GFTFSHAW |
| 3 | HAWMH |
| 4 | QIKAKSDDYATYYAESVKG |
| 5 | IKAKSDDYAT |
| 6 | TCWEWDLDF |
| 7 | WEWDLDF |
| 8 | TSWEWDLDF |
| 9 | RSSQSLLDSDGHTYLN |
| 10 | QSLLDSDGHTY |
| 11 | SVSNLES |
| 12 | SYS |
| 13 | MQATHAPPYT |
| 14 | EVQLVESGGGLVKPGGSLKLSCAASGFTFSHAWMHWVRQAPGKGLEWVAQIKAKSDDYATYYAESVKGRFTISRD |
| | DSKNTLYLQMNSLKTEDTAVYYCTCWEWDLDFWGQGTLVTVSS |
| 15 | QVQLQESGGGLVKPGGSLRLSCAASGFTFSHAWMHWVRQAPGKGLEWVGQIKAKSDDYATYYAESVKGRFTISR |
| | DDSKNTLYLQMNSLKTEDTAVYYCTCWEWDLDFWGQGTMVTVSS |
| 16 | EVQLVESGGGVVQPGRSLRLSCAASGFTFSHAWMHWVRQAPGKGLEWVAQIKAKSDDYATYYAESVKGRFSISR |
| | DNAKNSLYLQMNSLRVEDTVVYYCTCWEWDLDFWGQGTTVTVSS |
| 17 | EVQLMESGGGLVKPGGSLRLSCATSGFTFSHAWMHWVRQAPGKGLEWVGQIKAKSDDYATYYAESVKGRFTISR |
| | DDSKSTLFLQMNNLKTEDTAVYYCTCWEWDLDFWGQGTLVTVSS |
| 18 | DVVMTQSPLSLPVTLGQPASISCRSSQSLLDSDGHTYLNWFQQRPGQSPRRLIYSVSNLESGVPDRFSGSGSG |
| | TDFTLKISRVEAEDVGLYYCMQATHAPPYTFGQGTKLEIK |
| 19 | DIVMTQTPLSSPVTLGQPASISCRSSQSLLDSDGHTYLNWLQQRPGQPPRLLIYSVSNLESGVPDRFSGSGAGTDFTL |
| | KISRVEAEDVGVYYCMQATHAPPYTFGQGTKLEIK |
| 20 | DIVMTQTPLSLSVTPGQPASISCRSSQSLLDSDGHTYLNWLLQKPGQPPQLLIYSVSNLESGVPNRFSGSGSGTDFTL |
| | KISRVEAEDVGLYYCMQATHAPPYTFGGGTKVEIK |
| 21 | DVVMTQSPLSQPVTLGQPASISCRSSQSLLDSDGHTYLNWLQQRPGQSPRRLIYSVSNLESGVPDRFNGSGSGTDF |
| | TLSISRVEAEDVGVYYCMQATHAPPYTFGQGTKVEIK |
| 22 | MGWTLVFLFLLSVTAGVHSEVQLVESGGGLVKPGGSLKLSCAASGFTFSHAWMHWVRQAPGKGLEWVAQIKAK |
| | SDDYATYYAESVKGRFTISRDDSKNTLYLQMNSLKTEDTAVYYCTCWEWDLDFWGQGTLVTVSSASTKGPSVFPLA |
| | PSSKSTSGGTAALGCLVKDYFPEPVTVSWNSGALTSGVHTFPAVLQSSGLYSLSSVVTVPSSSLGTQTYICNVNHKPS |
| | NTKVDKKVEPKSCDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQVYTLPPSRDELTK |
| | NQVSLTCLVKGFYPSDIAVEWESNGQPENNYKTTPPVLDSDGSFFLYSKLTVDKSRWQQGNVFSCSVMHEALHNH |
| | YTQKSLSLSPGK |
| 23 | MGWTLVFLFLLSVTAGVHSQVQLQESGGGLVKPGGSLRLSCAASGFTFSHAWMHWVRQAPGKGLEWVGQIKAK |
| | SDDYATYYAESVKGRFTISRDDSKNTLYLQMNSLKTEDTAVYYCTCWEWDLDFWGQGTMVTVSSASTKGPSVFPL |
| | APSSKSTSGGTAALGCLVKDYFPEPVTVSWNSGALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQVYTLPPSRDELTK |
| | NQVSLTCLVKGFYPSDIAVEWESNGQPENNYKTTPPVLDSDGSFFLYSKLTVDKSRWQQGNVFSCSVMHEALHNH |
| | YTQKSLSLSPGK |
| 24 | MGWTLVFLFLLSVTAGVHSEVQLVESGGGVVQPGRSLRLSCAASGFTFSHAWMHWVRQAPGKGLEWVAQIKAK |
| | SDDYATYYAESVKGRFSISRDNAKNSLYLQMNSLRVEDTVVYYCTCWEWDLDFWGQGTTVTVSSASTKGPSVFPL |
| | APSSKSTSGGTAALGCLVKDYFPEPVTVSWNSGALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQVYTLPPSRDELTK |
| | NQVSLTCLVKGFYPSDIAVEWESNGQPENNYKTTPPVLDSDGSFFLYSKLTVDKSRWQQGNVFSCSVMHEALHNH |
| | YTQKSLSLSPGK |
| 25 | MGWTLVFLFLLSVTAGVHSEVQLMESGGGLVKPGGSLRLSCATSGFTFSHAWMHWVRQAPGKGLEWVGQIKAK |
| | SDDYATYYAESVKGRFTISRDDSKSTLFLQMNNLKTEDTAVYYCTCWEWDLDFWGQGTLVTVSSASTKGPSVFPLA |
| | PSSKSTSGGTAALGCLVKDYFPEPVTVSWNSGALTSGVHTFPAVLQSSGLYSLSSVVTVPSSSLGTQTYICNVNHKPS |
| | NTKVDKKVEPKSCDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQVYTLPPSRDELTK |
| | NQVSLTCLVKGFYPSDIAVEWESNGQPENNYKTTPPVLDSDGSFFLYSKLTVDKSRWQQGNVFSCSVMHEALHNH |
| | YTQKSLSLSPGK |
| 26 | MVSSAQFLGLLLLCFQGTRCDVVMTQSPLSLPVTLGQPASISCRSSQSLLDSDGHTYLNWFQQRPGQSPRRLIYSVS |
| | NLESGVPDRFSGSGSGTDFTLKISRVEAEDVGLYYCMQATHAPPYTFGQGTKLEIKRTVAAPSVFIFPPSDEQLKSGT |
| | ASVVCLLNNFYPREAKVQWKVDNALQSGNSQESVTEQDSKDSTYSLSSTLTLSKADYEKHKVYACEVTHQGLSSPV |
| | TKSFNRGEC |
| 27 | MVSSAQFLGLLLLCFQGTRCDIVMTQTPLSSPVTLGQPASISCRSSQSLLDSDGHTYLNWLQQRPGQPPRLLIYSVS |
| | NLESGVPDRFSGSGAGTDFTLKISRVEAEDVGVYYCMQATHAPPYTFGQGTKLEIKRTVAAPSVFIFPPSDEQLKSGT |
| | ASVVCLLNNFYPREAKVQWKVDNALQSGNSQESVTEQDSKDSTYSLSSTLTLSKADYEKHKVYACEVTHQGLSSPV |
| | TKSFNRGEC |
| 28 | MVSSAQFLGLLLLCFQGTRCDIVMTQTPLSLSVTPGQPASISCRSSQSLLDSDGHTYLNWLLQKPGQPPQLLIYSVSN |
| | LESGVPNRFSGSGSGTDFTLKISRVEAEDVGLYYCMQATHAPPYTFGGGTKVEIKRTVAAPSVFIFPPSDEQLKSGTA |
| | SVVCLLNNFYPREAKVQWKVDNALQSGNSQESVTEQDSKDSTYSLSSTLTLSKADYEKHKVYACEVTHQGLSSPVTK |
| | SFNRGEC |
| 29 | MVSSAQFLGLLLLCFQGTRCDVVMTQSPLSQPVTLGQPASISCRSSQSLLDSDGHTYLNWLQQRPGQSPRRLIYSVS |
| | NLESGVPDRFSGSGSGTDFTLSISRVEAEDVGVYYCMQATHAPPYTFGQGTKVEIKRTVAAPSVFIFPPSDEQLKSGT |
| | ASVVCLLNNFYPREAKVQWKVDNALQSGNSQESVTEQDSKDSTYSLSSTLTLSKADYEKHKVYACEVTHQGLSSPV |
| | TKSFNRGEC |
| 30 | EVQLVESGGGLVKPGGSLKLSCAASGFTFSHAWMHWVRQAPGKGLEWVAQIKAKSDDYATYYAESVKGRFTISRD |
| | DSKNTLYLQMNSLKTEDTAVYYCTCWEWDLDFWGQGTLVTVSSASTKGPSVFPLAPSSKSTSGGTAALGCLVKDYF |
| | PEPVTVSWNSGALTSGVHTFPAVLQSSGLYSLSSVVTVPSSSLGTQTYICNVNHKPSNTKVDKKVEPKSCDKTHTCP |
| | PCPAPELLGGPSVFLFPPKPKDTLMISRTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPSRDELTKNQVSLTCLVKGFYPSDIAVEW |
| | ESNGQPENNYKTTPPVLDSDGSFFLYSKLTVDKSRWQQGNVFSCSVMHEALHNHYTQKSLSLSPGK |
| 31 | QVQLQESGGGLVKPGGSLRLSCAASGFTFSHAWMHWVRQAPGKGLEWVGQIKAKSDDYATYYAESVKGRFTISR |
| | DDSKNTLYLQMNSLKTEDTAVYYCTCWEWDLDFWGQGTMVTVSSASTKGPSVFPLAPSSKSTSGGTAALGCLVK |
| | DYFPEPVTVSWNSGALTSGVHTFPAVLQSSGLYSLSSVVTVPSSSLGTQTYICNVNHKPSNTKVDKKVEPKSCDKTH |
| | TCPPCPAPELLGGPSVFLFPPKPKDTLMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTLPPSRDELTKNQVSLTCLVKGFYPSDIA |
| | VEWESNGQPENNYKTTPPVLDSDGSFFLYSKLTVDKSRWQQGNVFSCSVMHEALHNHYTQKSLSLSPGK |
| 32 | EVQLVESGGGVVQPGRSLRLSCAASGFTFSHAWMHWVRQAPGKGLEWVAQIKAKSDDYATYYAESVKGRFSISR |
| | DNAKNSLYLQMNSLRVEDTVVYYCTCWEWDLDFWGQGTTVTVSSASTKGPSVFPLAPSSKSTSGGTAALGCLVKD |
| | YFPEPVTVSWNSGALTSGVHTFPAVLQSSGLYSLSSVVTVPSSSLGTQTYICNVNHKPSNTKVDKKVEPKSCDKTHTC |
| | PPCPAPELLGGPSVFLFPPKPKDTLMISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPPSRDELTKNQVSLTCLVKGFYPSDIAVE |
| | WESNGQPENNYKTTPPVLDSDGSFFLYSKLTVDKSRWQQGNVFSCSVMHEALHNHYTQKSLSLSPGK |
| 33 | EVQLMESGGGLVKPGGSLRLSCATSGFTFSHAWMHWVRQAPGKGLEWVGQIKAKSDDYATYYAESVKGRFTISR |
| | DDSKSTLFLQMNNLKTEDTAVYYCTCWEWDLDFWGQGTLVTVSSASTKGPSVFPLAPSSKSTSGGTAALGCLVKD |
| | YFPEPVTVSWNSGALTSGVHTFPAVLQSSGLYSLSSVVTVPSSSLGTQTYICNVNHKPSNTKVDKKVEPKSCDKTHTC |
| | PPCPAPELLGGPSVFLFPPKPKDTLMISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPPSRDELTKNQVSLTCLVKGFYPSDIAVE |
| | WESNGQPENNYKTTPPVLDSDGSFFLYSKLTVDKSRWQQGNVFSCSVMHEALHNHYTQKSLSLSPGK |
| 34 | DVVMTQSPLSLPVTLGQPASISCRSSQSLLDSDGHTYLNWFQQRPGQSPRRLIYSVSNLESGVPDRFSGSGSGTDFT |
| | LKISRVEAEDVGLYYCMQATHAPPYTFGQGTKLEIKRTVAAPSVFIFPPSDEQLKSGTASVVCLLNNFYPREAKVQW |
| | KVDNALQSGNSQESVTEQDSKDSTYSLSSTLTLSKADYEKHKVYACEVTHQGLSSPVTKSFNRGEC |
| 35 | DIVMTQTPLSSPVTLGQPASISCRSSQSLLDSDGHTYLNWLQQRPGQPPRLLIYSVSNLESGVPDRFSGSGAGTDFTL |
| | KISRVEAEDVGVYYCMQATHAPPYTFGQGTKLEIKRTVAAPSVFIFPPSDEQLKSGTASVVCLLNNFYPREAKVQWK |
| | VDNALQSGNSQESVTEQDSKDSTYSLSSTLTLSKADYEKHKVYACEVTHQGLSSPVTKSFNRGEC |
| 36 | DIVMTQTPLSLSVTPGQPASISCRSSQSLLDSDGHTYLNWLLQKPGQPPQLLIYSVSNLESGVPNRFSGSGSGTDFTL |
| | KISRVEAEDVGLYYCMQATHAPPYTFGGGTKVEIKRTVAAPSVFIFPPSDEQLKSGTASVVCLLNNFYPREAKVQWK |
| | VDNALQSGNSQESVTEQDSKDSTYSLSSTLTLSKADYEKHKVYACEVTHQGLSSPVTKSFNRGEC |
| 37 | DVVMTQSPLSQPVTLGQPASISCRSSQSLLDSDGHTYLNWLQQRPGQSPRRLIYSVSNLESGVPDRFSGSGSGTDFT |
| | LSISRVEAEDVGVYYCMQATHAPPYTFGQGTKVEIKRTVAAPSVFIFPPSDEQLKSGTASVVCLLNNFYPREAKVQW |
| | KVDNALQSGNSQESVTEQDSKDSTYSLSSTLTLSKADYEKHKVYACEVTHQGLSSPVTKSFNRGEC |
| 38 | QVQLQESGGGLVKPGGSLRLSCAASGFTFSHAWMHWVRQAPGKGLEWVGQIKAKSDDYATYYAESVKGRFTISR |
| | DDSKNTLYLQMNSLKTEDTAVYYCTSWEWDLDFWGQGTMVTVSS |
| 39 | QVQLQESGGGLVKPGGSLRLSCAASGFTFSHAWMHWVRQAPGKGLEWVGQIKAKSDDYATYYAESVKGRFTISR |
| | DDSKNTLYLQMNSLKTEDTAVYYCTSWEWDLDFWGQGTMVTVSSASTKGPSVFPLAPSSKSTSGGTAALGCLVKD |
| | YFPEPVTVSWNSGALTSGVHTFPAVLQSSGLYSLSSVVTVPSSSLGTQTYICNVNHKPSNTKVDKKVEPKSCDKTHTC |
| | PPCPAPELLGGPSVFLFPPKPKDTLMISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYTQKSLSLSPGK |
| 40 | SPLPITPVNATCAIRHPCHNNLMNQIRSQLAQLNGSANALFILYYTAQGEPFPNNLDKLCGPNVTDFPPFHANGTEK |
| | AKLVELYRIVVYLGTSLGNITRDQKILNPSALSLHSKLNATADILRGLLSNVLCRLCSKYHVGHVDVTYGPDTSGKDVF |
| | QKKKLGCQLLGKYKQIIAVLAQAF |
| 41 | EQLEESVGDLVKPGASLTLTCTASGFSFSGLYYMCWVRQAPGKGLEWIACIWTGSTDSTYYATWAKGRFTISKTSST |
| | TVTLQMTSLTVADTATYFCARGGGVPGDGYALWGPGTLVTVSS |
| 42 | GTELVMTQTPASVSEPVGGTVTINCQASEDISSNLVWYQQKSGQPPKLLIYDASMLASGVPSRFKGSGSGTQFTLTI |
| | SDLECADGATYYCQSYYVASSSYFVNGFGGGTEVV |

What is claimed is:

1. A method of treating an individual with cancer with a therapeutic anti-leukemia inhibitory factor (LIF) antibody comprising determining a level of LIF that exceeds a reference level in a biological sample from the individual, and administering a therapeutic amount of the anti-LIF antibody to the individual when the level of LIF is greater than the reference level of LIF.

2. The method of claim 1, wherein the therapeutic anti-LIF antibody comprises:
a) an immunoglobulin heavy chain complementarity determining region 1 (VH-CDR1) comprising the amino acid sequence set forth in any one of SEQ ID NOs: 1-3;
b) an immunoglobulin heavy chain complementarity determining region 2 (VH-CDR2) comprising the amino acid sequence set forth in any one of SEQ ID NOs: 4 or 5;
c) an immunoglobulin heavy chain complementarity determining region 3 (VH-CDR3) comprising the amino acid sequence set forth in any one of SEQ ID NOs: 6-8;
d) an immunoglobulin light chain complementarity determining region 1 (VL-CDR1) comprising the amino acid sequence set forth in any one of SEQ ID NOs: 9 or 10;
e) an immunoglobulin light chain complementarity determining region 2 (VL-CDR2) comprising the amino acid sequence set forth in any one of SEQ ID NOs: 11 or 12; and
f) an immunoglobulin light chain complementarity determining region 3 (VL-CDR3) comprising the amino acid sequence set forth in SEQ ID NO: 13.

3. The method of claim 2, wherein the therapeutic anti-LIF antibody comprises an immunoglobulin heavy chain variable region comprising at least 85%, 90%, 95%, 97%, 98%, 99%, or 100% identity to SEQ ID NOs: 14, 15, 17, or 38 and an immunoglobulin light chain variable region comprising at least 85%, 90%, 95%, 97%, 98%, 99%, or 100% identity to SEQ ID NO: 18-21.

4. The method of claim 3, wherein the therapeutic anti-LIF antibody comprises an immunoglobulin heavy chain region comprising at least 85%, 90%, 95%, 97%, 98%, 99%, or 100% identity to SEQ ID NOs: 30-33 or 39, and an immunoglobulin light chain region comprising at least 85%, 90%, 95%, 97%, 98%, 99%, or 100% identity to SEQ ID NOs: 34-37.

5. The method of any one of claims 1 to 4, wherein the therapeutic anti-LIF antibody is an IgG antibody comprising two immunoglobulin heavy chains and two immunoglobulin light chains.

6. The method of any one of claims 1 to 4, wherein the therapeutic anti-LIF antibody is humanized.

7. The method of any one of claims 1 to 6, wherein the level of LIF is a LIF protein level and determining the level comprises performing at least one assay that detects LIF protein or receiving the results of at least one assay that detects LIF protein.

8. The method of claim 7, wherein the at least one assay comprises immunohistochemistry.

9. The method of claim 8, wherein the reference level is about 1%, 2%, 3%. 4%, 5%, 6%, 7%, 8%, 9%, 10%, 15%, 20%, 25%, 30%, or 35% of cells staining positive with an anti-LIF antibody.

10. The method of claim 8, wherein the reference level is or exceeds an IHC-score of about 10 to about 100.

11. The method of claim 7, wherein the at least one assay comprises enzyme linked immunosorbent assay (ELISA).

12. The method of claim 11, wherein the ELISA detects electrochemiluminescence.

13. The method of claim 11 or 12, wherein the reference level is about 4 pg/mL of LIF in an undiluted biological sample from the individual.

14. The method of any one of claims 7 to 13, wherein the reference level of LIF corresponds to the 5^{th} percentile, 10^{th} percentile, 25^{th} percentile, or the 50^{th} percentile of LIF protein expression in LIF positive human cancers of the same type.

15. The method of any one of claims 7 to 13, wherein the reference level of LIF corresponds to the 5^{th} percentile, 10^{th} percentile, 25^{th} percentile, or the 50^{th} percentile of LIF protein expression in human cancer.

16. The method of claim 15, wherein the human cancer is selected from the list consisting of lung cancer, ovarian cancer, kidney cancer, bladder cancer, pancreatic cancer, prostate cancer, genitourinary cancer, gynecologic cancer, gastrointestinal cancer, endocrine system cancer, glioblastoma multiforme, breast cancer, melanoma, colorectal cancer, bile duct cancer, cervical cancer, endometrial cancer, head and neck squamous cell carcinoma, and combinations thereof.

17. The method of claim 16, wherein the human cancer is selected from the list consisting of non-small cell lung cancer, glioblastoma multiforme, epithelial ovarian carcinoma, pancreatic adenocarcinoma, and combinations thereof.

18. The method of any one of claims 1 to 17, wherein the biological sample comprises a blood sample.

19. The method of claim 18, wherein the blood sample is plasma.

20. The method of claim 18, wherein the blood sample is serum.

21. The method of any one of claims 1 to 18, wherein the biological sample comprises a tissue sample.

22. The method of claim 21, wherein the biological sample is a tumor biopsy.

23. The method of any one of claims 1 to 22, wherein the method further comprises determining a protein level of an immunomodulatory molecule that exceeds a reference level of the immunomodulatory molecule.

24. The method of claim 23, wherein the immunomodulatory molecule is selected from CCL7, CCL2, CCL3, and CCL22.

25. The method of any one of claims 1 to 24, wherein the method further comprises determining a protein level of an immunomodulatory molecule that is below a reference level of the immunomodulatory molecule.

26. The method of claim 25, wherein the immunomodulatory molecule is selected from WICK CXCL9, CXCL10, CXCR3, and PD-L1.

27. The method of any one of claims 1 to 26, wherein the method further comprises determining a level of a Type II macrophage (M2) marker that exceeds a reference level of protein of the Type II macrophage (M2) marker.

28. The method of claim 27, wherein the M2 marker is selected from the list consisting of CD206, CD163, PF4, CTSK, and ARG1.


SEQUENCE LISTING (42 sequences)

SEQ ID NO: 1 - protein, 10 aa, Artificial Sequence
  Synthetic Peptide
GFTFSHAWMH

SEQ ID NO: 2 - protein, 8 aa, Artificial Sequence
  Synthetic Peptide
GFTFSHAW

SEQ ID NO: 3 - protein, 5 aa, Artificial Sequence
  Synthetic Peptide
HAWMH

SEQ ID NO: 4 - protein, 19 aa, Artificial Sequence
  Synthetic Peptide
QIKAKSDDYATYYAESVKG

SEQ ID NO: 5 - protein, 10 aa, Artificial Sequence
  Synthetic Peptide
IKAKSDDYAT

SEQ ID NO: 6 - protein, 9 aa, Artificial Sequence
  Synthetic Peptide
TCWEWDLDF

SEQ ID NO: 7 - protein, 7 aa, Artificial Sequence
  Synthetic Peptide
WEWDLDF

SEQ ID NO: 8 - protein, 9 aa, Artificial Sequence
  Synthetic Peptide
TSWEWDLDF

SEQ ID NO: 9 - protein, 16 aa, Artificial Sequence
  Synthetic Peptide
RSSQSLLDSDGHTYLN

SEQ ID NO: 10 - protein, 11 aa, Artificial Sequence
  Synthetic Peptide
QSLLDSDGHTY

SEQ ID NO: 11 - protein, 7 aa, Artificial Sequence
  Synthetic Peptide
SVSNLES

SEQ ID NO: 12 - protein, 3 aa, Artificial Sequence
  Synthetic Peptide
SVS

SEQ ID NO: 13 - protein, 10 aa, Artificial Sequence
  Synthetic Peptide
MQATHAPPYT

SEQ ID NO: 14 - protein, 118 aa, Artificial Sequence
  Synthetic Peptide
EVQLVESGGGLVKPGGSLKLSCAASGFTFSHAWMHWVRQAPGKGLEWVAQIKAKSDDYATYYAESVKGRFTISRDDSKNTLYLQMNSLKTEDTAVYYCTCWEWDLDFWGQGTLVTVSS

SEQ ID NO: 15 - protein, 118 aa, Artificial Sequence
  Synthetic Peptide
QVQLQESGGGLVKPGGSLRLSCAASGFTFSHAWMHWVRQAPGKGLEWVGQIKAKSDDYATYYAESVKGRFTISRDDSKNTLYLQMNSLKTEDTAVYYCTCWEWDLDFWGQGTMVTVSS

SEQ ID NO: 16 - protein, 118 aa, Artificial Sequence
  Synthetic Peptide
EVQLVESGGGVVQPGRSLRLSCAASGFTFSHAWMHWVRQAPGKGLEWVAQIKAKSDDYATYYAESVKGRFSISRDNAKNSLYLQMNSLRVEDTVVYYCTCWEWDLDFWGQGTTVTVSS

SEQ ID NO: 17 - protein, 118 aa, Artificial Sequence
  Synthetic Peptide
EVQLMESGGGLVKPGGSLRLSCATSGFTFSHAWMHWVRQAPGKGLEWVGQIKAKSDDYATYYAESVKGRFTISRDDSKSTLFLQMNNLKTEDTAVYYCTCWEWDLDFWGQGTLVTVSS

SEQ ID NO: 18 - protein, 113 aa, Artificial Sequence
  Synthetic Peptide
DVVMTQSPLSLPVTLGQPASISCRSSQSLLDSDGHTYLNWFQQRPGQSPRRLIYSVSNLESGVPDRFSGSGSGTDFTLKISRVEAEDVGLYYCMQATHAPPYTFGQGTKLEIK

SEQ ID NO: 19 - protein, 113 aa, Artificial Sequence
  Synthetic Peptide
DIVMTQTPLSSPVTLGQPASISCRSSQSLLDSDGHTYLNWLQQRPGQPPRLLIYSVSNLESGVPDRFSGSGAGTDFTLKISRVEAEDVGVYYCMQATHAPPYTFGQGTKLEIK

SEQ ID NO: 20 - protein, 113 aa, Artificial Sequence
  Synthetic Peptide
DIVMTQTPLSLSVTPGQPASISCRSSQSLLDSDGHTYLNWLLQKPGQPPQLLIYSVSNLESGVPNRFSGSGSGTDFTLKISRVEAEDVGLYYCMQATHAPPYTFGGGTKVEIK

SEQ ID NO: 21 - protein, 113 aa, Artificial Sequence
  Synthetic Peptide
DVVMTQSPLSQPVTLGQPASISCRSSQSLLDSDGHTYLNWLQQRPGQSPRRLIYSVSNLESGVPDRFNGSGSGTDFTLSISRVEAEDVGVYYCMQATHAPPYTFGQGTKVEIK

SEQ ID NO: 22 - protein, 467 aa, Artificial Sequence
  Synthetic Peptide
MGWTLVFLFLLSVTAGVHSEVQLVESGGGLVKPGGSLKLSCAASGFTFSHAWMHWVRQAPGKGLEWVAQIKAKSDDYATYYAESVKGRFTISRDDSKNTLYLQMNSLKTEDTAVYYCTCWEWDLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 467 aa, Artificial Sequence
  Synthetic Peptide
MGWTLVFLFLLSVTAGVHSQVQLQESGGGLVKPGGSLRLSCAASGFTFSHAWMHWVRQAPGKGLEWVGQIKAKSDDYATYYAESVKGRFTISRDDSKNTLYLQMNSLKTEDTAVYYCTCWEWDLDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 467 aa, Artificial Sequence
  Synthetic Peptide
MGWTLVFLFLLSVTAGVHSEVQLVESGGGVVQPGRSLRLSCAASGFTFSHAWMHWVRQAPGKGLEWVAQIKAKSDDYATYYAESVKGRFSISRDNAKNSLYLQMNSLRVEDTVVYYCTCWEWDLDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 467 aa, Artificial Sequence
  Synthetic Peptide
MGWTLVFLFLLSVTAGVHSEVQLMESGGGLVKPGGSLRLSCATSGFTFSHAWMHWVRQAPGKGLEWVGQIKAKSDDYATYYAESVKGRFTISRDDSKSTLFLQMNNLKTEDTAVYYCTCWEWDLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 240 aa, Artificial Sequence
  Synthetic Peptide
MVSSAQFLGLLLLCFQGTRCDVVMTQSPLSLPVTLGQPASISCRSSQSLLDSDGHTYLNWFQQRPGQSPRRLIYSVSNLESGVPDRFSGSGSGTDFTLKISRVEAEDVGLYYCMQATHAPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 240 aa, Artificial Sequence
  Synthetic Peptide
MVSSAQFLGLLLLCFQGTRCDIVMTQTPLSSPVTLGQPASISCRSSQSLLDSDGHTYLNWLQQRPGQPPRLLIYSVSNLESGVPDRFSGSGAGTDFTLKISRVEAEDVGVYYCMQATHAPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 240 aa, Artificial Sequence
  Synthetic Peptide
MVSSAQFLGLLLLCFQGTRCDIVMTQTPLSLSVTPGQPASISCRSSQSLLDSDGHTYLNWLLQKPGQPPQLLIYSVSNLESGVPNRFSGSGSGTDFTLKISRVEAEDVGLYYCMQATHAPPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 240 aa, Artificial Sequence
  Synthetic Peptide
MVSSAQFLGLLLLCFQGTRCDVVMTQSPLSQPVTLGQPASISCRSSQSLLDSDGHTYLNWLQQRPGQSPRRLIYSVSNLESGVPDRFSGSGSGTDFTLSISRVEAEDVGVYYCMQATHAPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 448 aa, Artificial Sequence
  Synthetic Peptide
EVQLVESGGGLVKPGGSLKLSCAASGFTFSHAWMHWVRQAPGKGLEWVAQIKAKSDDYATYYAESVKGRFTISRDDSKNTLYLQMNSLKTEDTAVYYCTCWEWDLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 448 aa, Artificial Sequence
  Synthetic Peptide
QVQLQESGGGLVKPGGSLRLSCAASGFTFSHAWMHWVRQAPGKGLEWVGQIKAKSDDYATYYAESVKGRFTISRDDSKNTLYLQMNSLKTEDTAVYYCTCWEWDLDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 448 aa, Artificial Sequence
  Synthetic Peptide
EVQLVESGGGVVQPGRSLRLSCAASGFTFSHAWMHWVRQAPGKGLEWVAQIKAKSDDYATYYAESVKGRFSISRDNAKNSLYLQMNSLRVEDTVVYYCTCWEWDLDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 448 aa, Artificial Sequence
  Synthetic Peptide
EVQLMESGGGLVKPGGSLRLSCATSGFTFSHAWMHWVRQAPGKGLEWVGQIKAKSDDYATYYAESVKGRFTISRDDSKSTLFLQMNNLKTEDTAVYYCTCWEWDLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 220 aa, Artificial Sequence
  Synthetic Peptide
DVVMTQSPLSLPVTLGQPASISCRSSQSLLDSDGHTYLNWFQQRPGQSPRRLIYSVSNLESGVPDRFSGSGSGTDFTLKISRVEAEDVGLYYCMQATHAPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 220 aa, Artificial Sequence
  Synthetic Peptide
DIVMTQTPLSSPVTLGQPASISCRSSQSLLDSDGHTYLNWLQQRPGQPPRLLIYSVSNLESGVPDRFSGSGAGTDFTLKISRVEAEDVGVYYCMQATHAPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 220 aa, Artificial Sequence
  Synthetic Peptide
DIVMTQTPLSLSVTPGQPASISCRSSQSLLDSDGHTYLNWLLQKPGQPPQLLIYSVSNLESGVPNRFSGSGSGTDFTLKISRVEAEDVGLYYCMQATHAPPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 220 aa, Artificial Sequence
  Synthetic Peptide
DVVMTQSPLSQPVTLGQPASISCRSSQSLLDSDGHTYLNWLQQRPGQSPRRLIYSVSNLESGVPDRFSGSGSGTDFTLSISRVEAEDVGVYYCMQATHAPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 118 aa, Artificial Sequence
  Synthetic Peptide
QVQLQESGGGLVKPGGSLRLSCAASGFTFSHAWMHWVRQAPGKGLEWVGQIKAKSDDYATYYAESVKGRFTISRDDSKNTLYLQMNSLKTEDTAVYYCTSWEWDLDFWGQGTMVTVSS

SEQ ID NO: 39 - protein, 448 aa, Artificial Sequence
  Synthetic Peptide
QVQLQESGGGLVKPGGSLRLSCAASGFTFSHAWMHWVRQAPGKGLEWVGQIKAKSDDYATYYAESVKGRFTISRDDSKNTLYLQMNSLKTEDTAVYYCTSWEWDLDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 180 aa, Artificial Sequence
  Synthetic Peptide
SPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

SEQ ID NO: 41 - protein, 120 aa, Artificial Sequence
  Synthetic Peptide
EQLEESVGDLVKPGASLTLTCTASGFSFSGLYYMCWVRQAPGKGLEWIACIWTGSTDSTYYATWAKGRFTISKTSSTTVTLQMTSLTVADTATYFCARGGGVPGDGYALWGPGTLVTVSS

SEQ ID NO: 42 - protein, 112 aa, Artificial Sequence
  Synthetic Peptide
GTELVMTQTPASVSEPVGGTVTINCQASEDISSNLVWYQQKSGQPPKLLIYDASMLASGVPSRFKGSGSGTQFTLTISDLECADGATYYCQSYYVASSSYFVNGFGGGTEVV